SEQUENCE LISTING (76 sequences)

SEQ ID NO: 1 - protein, 735 aa, synthetic construct [sequence omitted]

SEQ ID NO: 2 - protein, 6 aa, synthetic construct
EGSGRN

SEQ ID NO: 3 - protein, 7 aa, synthetic construct
AEGSGRN

SEQ ID NO: 4 - protein, 8 aa, synthetic construct
LAEGSGRN

SEQ ID NO: 5 - protein, 9 aa, synthetic construct
LAEGSGRNA

SEQ ID NO: 6 - DNA, 18 bp, synthetic construct
  source 1..18: mol_type = other DNA
gagggcagcggccggaac

SEQ ID NO: 7 - DNA, 21 bp, synthetic construct
  source 1..21: mol_type = other DNA
gcagagggcagcggccggaac

SEQ ID NO: 8 - DNA, 24 bp, synthetic construct
  source 1..24: mol_type = other DNA
ctagcagagggcagcggccggaac

SEQ ID NO: 9 - DNA, 27 bp, synthetic construct
  source 1..27: mol_type = other DNA
ctagcagagggcagcggccggaacgct

SEQ ID NO: 10 - protein, 735 aa, synthetic construct
  VARIANT 67: note = E or A
  VARIANT 708: note = V or I
MAADGYLPDWLEDTLSEGIRQWWKLKPGPPPPKPAERHKDDSRGLVLPGYKYLGPFNGLDKGEPVNXADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSXNVDFTVDTNGVYSEPRPIGTRYLTRNL

SEQ ID NO: 11 - protein, 741 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNEGSGRNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 12 - protein, 742 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNAEGSGRNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 13 - protein, 743 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLAEGSGRNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 14 - protein, 744 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLAEGSGRNARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 15 - protein, 741 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNEGSGRNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 16 - protein, 742 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNAEGSGRNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 17 - protein, 743 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLAEGSGRNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 18 - protein, 744 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLAEGSGRNARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 19 - protein, 741 aa, synthetic construct
MAADGYLPDWLEDTLSEGIRQWWKLKPGPPPPKPAERHKDDSRGLVLPGYKYLGPFNGLDKGEPVNAADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNEGSGRNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 20 - protein, 742 aa, synthetic construct
MAADGYLPDWLEDTLSEGIRQWWKLKPGPPPPKPAERHKDDSRGLVLPGYKYLGPFNGLDKGEPVNAADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNAEGSGRNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 21 - protein, 743 aa, synthetic construct
MAADGYLPDWLEDTLSEGIRQWWKLKPGPPPPKPAERHKDDSRGLVLPGYKYLGPFNGLDKGEPVNAADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLAEGSGRNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 22 - protein, 744 aa, synthetic construct
MAADGYLPDWLEDTLSEGIRQWWKLKPGPPPPKPAERHKDDSRGLVLPGYKYLGPFNGLDKGEPVNAADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLAEGSGRNARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 23 - protein, 741 aa, synthetic construct
MAADGYLPDWLEDTLSEGIRQWWKLKPGPPPPKPAERHKDDSRGLVLPGYKYLGPFNGLDKGEPVNAADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNEGSGRNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 24 - protein, 742 aa, synthetic construct
MAADGYLPDWLEDTLSEGIRQWWKLKPGPPPPKPAERHKDDSRGLVLPGYKYLGPFNGLDKGEPVNAADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNAEGSGRNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 25 - protein, 743 aa, synthetic construct
MAADGYLPDWLEDTLSEGIRQWWKLKPGPPPPKPAERHKDDSRGLVLPGYKYLGPFNGLDKGEPVNAADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLAEGSGRNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 26 - protein, 744 aa, synthetic construct
MAADGYLPDWLEDTLSEGIRQWWKLKPGPPPPKPAERHKDDSRGLVLPGYKYLGPFNGLDKGEPVNAADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLAEGSGRNARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 27 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQEGSGRNATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 28 - protein, 736 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAEGSGRNATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 29 - protein, 737 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQLAEGSGRNATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 30 - protein, 738 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQLAEGSGRNAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 31 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQEGSGRNATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 32 - protein, 736 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAEGSGRNATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 33 - protein, 737 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQLAEGSGRNATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 34 - protein, 738 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQLAEGSGRNAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 35 - protein, 735 aa, synthetic construct
MAADGYLPDWLEDTLSEGIRQWWKLKPGPPPPKPAERHKDDSRGLVLPGYKYLGPFNGLDKGEPVNAADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQEGSGRNATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 36 - protein, 736 aa, synthetic construct
MAADGYLPDWLEDTLSEGIRQWWKLKPGPPPPKPAERHKDDSRGLVLPGYKYLGPFNGLDKGEPVNAADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAEGSGRNATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 37 - protein, 737 aa, synthetic construct
MAADGYLPDWLEDTLSEGIRQWWKLKPGPPPPKPAERHKDDSRGLVLPGYKYLGPFNGLDKGEPVNAADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQLAEGSGRNATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 38 - protein, 738 aa, synthetic construct
MAADGYLPDWLEDTLSEGIRQWWKLKPGPPPPKPAERHKDDSRGLVLPGYKYLGPFNGLDKGEPVNAADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQLAEGSGRNAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 39 - protein, 735 aa, synthetic construct
MAADGYLPDWLEDTLSEGIRQWWKLKPGPPPPKPAERHKDDSRGLVLPGYKYLGPFNGLDKGEPVNAADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQEGSGRNATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 40 - protein, 736 aa, synthetic construct
MAADGYLPDWLEDTLSEGIRQWWKLKPGPPPPKPAERHKDDSRGLVLPGYKYLGPFNGLDKGEPVNAADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAEGSGRNATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 41 - protein, 737 aa, synthetic construct
MAADGYLPDWLEDTLSEGIRQWWKLKPGPPPPKPAERHKDDSRGLVLPGYKYLGPFNGLDKGEPVNAADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQLAEGSGRNATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 42 - protein, 738 aa, synthetic construct
MAADGYLPDWLEDTLSEGIRQWWKLKPGPPPPKPAERHKDDSRGLVLPGYKYLGPFNGLDKGEPVNAADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQLAEGSGRNAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 43 - protein, 741 aa, synthetic construct; identical to SEQ ID NO: 11

SEQ ID NO: 44 - protein, 742 aa, synthetic construct; identical to SEQ ID NO: 12

SEQ ID NO: 45 - protein, 743 aa, synthetic construct; identical to SEQ ID NO: 13

SEQ ID NO: 46 - protein, 744 aa, synthetic construct; identical to SEQ ID NO: 14

SEQ ID NO: 47 - protein, 741 aa, synthetic construct; identical to SEQ ID NO: 15

SEQ ID NO: 48 - protein, 742 aa, synthetic construct; identical to SEQ ID NO: 16

SEQ ID NO: 49 - protein, 743 aa, synthetic construct; identical to SEQ ID NO: 17

SEQ ID NO: 50 - protein, 744 aa, synthetic construct; identical to SEQ ID NO: 18

SEQ ID NO: 51 - protein, 741 aa, synthetic construct; identical to SEQ ID NO: 19

SEQ ID NO: 52 - protein, 742 aa, synthetic construct; identical to SEQ ID NO: 20

SEQ ID NO: 53 - protein, 743 aa, synthetic construct; identical to SEQ ID NO: 21

SEQ ID NO: 54 - protein, 744 aa, synthetic construct; identical to SEQ ID NO: 22

SEQ ID NO: 55 - protein, 741 aa, synthetic construct; identical to SEQ ID NO: 23

SEQ ID NO: 56 - protein, 742 aa, synthetic construct; identical to SEQ ID NO: 24

SEQ ID NO: 57 - protein, 743 aa, synthetic construct; identical to SEQ ID NO: 25

SEQ ID NO: 58 - protein, 744 aa, synthetic construct; identical to SEQ ID NO: 26

SEQ ID NO: 59 - protein, 735 aa, synthetic construct; identical to SEQ ID NO: 27

SEQ ID NO: 60 - protein, 736 aa, synthetic construct; identical to SEQ ID NO: 28

SEQ ID NO: 61 - protein, 737 aa, synthetic construct; identical to SEQ ID NO: 29

SEQ ID NO: 62 - protein, 738 aa, synthetic construct; identical to SEQ ID NO: 30

SEQ ID NO: 63 - protein, 735 aa, synthetic construct; identical to SEQ ID NO: 31

SEQ ID NO: 64 - protein, 736 aa, synthetic construct; identical to SEQ ID NO: 32

SEQ ID NO: 65 - protein, 737 aa, synthetic construct; identical to SEQ ID NO: 33

SEQ ID NO: 66 - protein, 738 aa, synthetic construct; identical to SEQ ID NO: 34

SEQ ID NO: 67 - protein, 735 aa, synthetic construct; identical to SEQ ID NO: 35

SEQ ID NO: 68 - protein, 736 aa, synthetic construct; identical to SEQ ID NO: 36

SEQ ID NO: 69 - protein, 737 aa, synthetic construct; identical to SEQ ID NO: 37

SEQ ID NO: 70 - protein, 738 aa, synthetic construct; identical to SEQ ID NO: 38

SEQ ID NO: 71 - protein, 735 aa, synthetic construct; identical to SEQ ID NO: 39

SEQ ID NO: 72 - protein, 736 aa, synthetic construct; identical to SEQ ID NO: 40

SEQ ID NO: 73 - protein, 737 aa, synthetic construct; identical to SEQ ID NO: 41

SEQ ID NO: 74 - protein, 738 aa, synthetic construct; identical to SEQ ID NO: 42

SEQ ID NO: 75 - protein, 621 aa, synthetic construct
TPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 76 - protein, 536 aa, synthetic construct
TPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

(19) United States
(12) Patent Application Publication
(10) Pub. No.: US 2025/0327092 A1
(43) Pub. Date: Oct. 23, 2025

(54) ADENO-ASSOCIATED VIRUS VECTORS FOR NUCLEIC ACID DELIVERY TO RETINAL CELLS

(71) Applicant: UNIVERSITY OF PITTSBURGH - OF THE COMMONWEALTH SYSTEM OF HIGHER EDUCATION, Pittsburgh, PA (US)

(72) Inventors: Leah Caroline THOMAS BYRNE, Pittsburgh, PA (US); Molly E. JOHNSON, Pittsburgh, PA (US); William Richard STAUFFER, Pittsburgh, PA (US); Bilge Esin OZTURK, Pittsburgh, PA (US)

(21) Appl. No.: 18/851,534

(22) PCT Filed: Mar. 30, 2023
(86) PCT No.: PCT/US2023/016871

§ 371 Date: Sep. 26, 2024

Related U.S. Application Data
(60) Provisional application No. 63/325,541, filed on Mar. 30, 2022.
(60) Provisional application No. 63/325,542, filed on Mar. 30, 2022.
(60) Provisional application No. 63/325,540, filed on Mar. 30, 2022.
(60) Provisional application No. 63/325,543, filed on Mar. 30, 2022.
(60) Provisional application No. 63/325,544, filed on Mar. 30, 2022.
(60) Provisional application No. 63/325,548, filed on Mar. 30, 2022.
(60) Provisional application No. 63/325,550, filed on Mar. 30, 2022.
(60) Provisional application No. 63/325,551, filed on Mar. 30, 2022.
(60) Provisional application No. 63/325,553, filed on Mar. 30, 2022.
(60) Provisional application No. 63/325,555, filed on Mar. 30, 2022.
(60) Provisional application No. 63/325,558, filed on Mar. 30, 2022.
(60) Provisional application No. 63/325,559, filed on Mar. 30, 2022.
(60) Provisional application No. 63/325,562, filed on Mar. 30, 2022.

(51) Int. Cl. C12N 15/86; A61K 48/00; A61P 27/02; C07K 14/005

(52) U.S. Cl. CPC C12N 15/86; A61K 48/0075; A61P 27/02; C07K 14/005; C12N 2750/14122; C12N 2750/14143

(57) ABSTRACT

This document relates to AAV vectors (e.g., AAV2 vectors). For example, AAV vectors (e.g., AAV2 vectors) containing an AAV capsid polypeptide that includes an amino acid sequence set forth in Table 1 (or a variant thereof) or Formula A, such AAV capsid polypeptides, nucleic acid molecules encoding such vectors, nucleic acid molecules encoding such AAV capsid polypeptides, host cells containing and/or expressing such nucleic acid molecules, and methods and materials for making or using such vectors and/or AAV capsid polypeptides are provided.

### CROSS-REFERENCE TO RELATED APPLICATIONS

This application claims the benefit of U.S. Patent Application Ser. No. 63/325,541, filed on Mar. 30, 2022, of U.S. Patent Application Ser. No. 63/325,543, filed on Mar. 30, 2022, of U.S. Patent Application Ser. No. 63/325,553, filed on Mar. 30, 2022, of U.S. Patent Application Ser. No. 63/325,562, filed on Mar. 30, 2022, of U.S. Patent Application Ser. No. 63/325,540, filed on Mar. 30, 2022, of U.S. Patent Application Ser. No. 63/325,542, filed on Mar. 30, 2022, of U.S. Patent Application Ser. No. 63/325,544, filed on Mar. 30, 2022, of U.S. Patent Application Ser. No. 63/325,548, filed on Mar. 30, 2022, of U.S. Patent Application Ser. No. 63/325,550, filed on Mar. 30, 2022, of U.S. Patent Application Ser. No. 63/325,551, filed on Mar. 30, 2022, of U.S. Patent Application Ser. No. 63/325,555, filed on Mar. 30, 2022, of U.S. Patent Application Ser. No. 63/325,558, filed on Mar. 30, 2022, and of U.S. Patent Application Ser. No. 63/325,559, filed on Mar. 30, 2022. The disclosures of the prior applications are considered part of (and are incorporated by reference in) the disclosure of this application.

### STATEMENT REGARDING FEDERAL FUNDING

This invention was made with government support under MH120094 awarded by the National Institutes of Health. The government has certain rights in the invention.

### TECHNICAL FIELD

This document relates to adeno-associated virus (AAV) vectors. For example, this document provides methods and materials for making and using AAV vectors (e.g., AAV2 vectors) having (a) the ability to deliver nucleic acid to foveal cones, (b) an increased efficiency to deliver nucleic acid to retinal cells, (c) the ability to deliver nucleic acid to retinal cells and drive high expression levels of nucleic acid within retinal cells, (d) the ability to deliver nucleic acid to retinal cells across retinal regions (e.g., across at least two retinal regions), (e) the ability to deliver nucleic acid to retinal cells of the parafovea region of the eye, (f) the ability to deliver nucleic acid to two or more different retinal cell types within an eye, (g) the ability to deliver nucleic acid to retinal pigment epithelial (RPE) cells, (h) an increased efficiency to deliver nucleic acid to photoreceptor cells of the retina, (i) an increased efficiency to deliver nucleic acid to retinal ganglion cells of the retina, (j) an increased efficiency to deliver nucleic acid to bipolar cells of the retina, (k) an increased efficiency to deliver nucleic acid to OFF-retinal ganglion cells, and/or (l) increased packaging efficiency and the ability to deliver nucleic acid to cells (e.g., retinal cells).

### BACKGROUND

Viral vectors, such as AAV vectors, are efficient vehicles for in vivo nucleic acid delivery, and their use in the clinic is expanding. Improved AAV vectors and AAV production techniques for making effective AAV vector preparations should further expand the use of AAV vectors in the laboratory and clinic.

### SUMMARY

This document provides AAV vectors (e.g., AAV2 vectors). For example, this document provides AAV vectors (e.g., AAV2 vectors) containing a capsid polypeptide that includes an amino acid sequence set forth in Table 1 (or a variant thereof) or Formula A. The AAV vectors (e.g., AAV2 vectors) described herein containing a capsid polypeptide that includes an amino acid sequence set forth in Table 1 (or a variant thereof) or Formula A can have the ability to infect retinal cells (e.g., retinal ganglion cells, photoreceptor cells, and bipolar cells) in vivo and deliver exogenous nucleic acid to the infected retinal cells such that the infected retinal cells express the exogenous nucleic acid. This document also provides methods and materials for making and using AAV vectors (e.g., AAV2 vectors) having the ability to deliver nucleic acid to retinal cells.

As described herein, an AAV vector (e.g., an AAV2 vector) provided herein having an AAV capsid polypeptide that includes an amino acid sequence set forth in Table 1 (or a variant thereof) or Formula A can have the ability to infect retinal cells (e.g., retinal ganglion cells, photoreceptor cells, and bipolar cells) in vivo and deliver exogenous nucleic acid to the infected retinal cells such that the infected retinal cells express the exogenous nucleic acid. In some cases, an AAV vector (e.g., an AAV2 vector) provided herein can have the ability to drive a level of mRNA expression of an exogenous nucleic acid in retinal cells (e.g., retinal ganglion cells, photoreceptor cells, and bipolar cells) of a mammal (e.g., a human or a non-human primate) that is greater than (e.g., at least 2 percent greater than, at least 2.5 percent greater than, at least 5 percent greater than, at least 7.5 percent greater than, at least 10 percent greater than, at least 25 percent greater than, at least 50 percent greater than, at least 75 percent greater than, or at least 100 percent greater than) the level of mRNA expression of an exogenous nucleic acid driven by a comparable AAV vector having an AAV capsid polypeptide that consists of the amino acid sequence of SEQ ID NO: 1 (e.g., a wild-type AAV2 vector) in retinal cells of a control mammal (e.g., a control human or a control non-human primate). In some cases, an AAV vector (e.g., an AAV2 vector) provided herein having an AAV capsid polypeptide that includes an amino acid sequence set forth in Table 1 (or a variant thereof) or Formula A can be used in place of the 7m8 AAV2 vector (Dalkara et al., Sci. Transl. Med., 5 (189): 189ra76 (2013) and Bennett et al., J. Struct. Biol., 209 (2): 107433 (2020)) or in place of the K912 AAV2 vector (Öztürk et al., eLife, 10: e64175 (2021)) to deliver nucleic acid to retinal cells (e.g., retinal ganglion cells, photoreceptor cells, and bipolar cells).

In another aspect, this document provides AAV vectors (e.g., AAV2 vectors). For example, this document provides AAV vectors (e.g., AAV2 vectors) containing a capsid polypeptide that includes an amino acid sequence set forth in Table 1 (or a variant thereof) or Formula A. The AAV vectors (e.g., AAV2 vectors) described herein containing a capsid polypeptide that includes an amino acid sequence set forth in Table 1 (or a variant thereof) or Formula A can have the ability to infect foveal cones in vivo and deliver exogenous nucleic acid to the infected foveal cones such that the infected foveal cones express the exogenous nucleic acid. This document also provides methods and materials for making and using AAV vectors (e.g., AAV2 vectors) having the ability to deliver nucleic acid to foveal cones.

As described herein, an AAV vector (e.g., an AAV2 vector) provided herein having an AAV capsid polypeptide that includes an amino acid sequence set forth in Table I (or a variant thereof) or Formula A can have the ability to infect foveal cones in vivo and deliver exogenous nucleic acid to the infected foveal cones such that the infected foveal cones express the exogenous nucleic acid. In some cases, an AAV vector (e.g., an AAV2 vector) provided herein can have the ability to infect and drive mRNA expression of an exogenous nucleic acid in cone cells present in the fovea of a mammal (e.g., a human or a non-human primate). In some cases, an AAV vector (e.g., an AAV2 vector) provided herein can have the ability to drive a level of mRNA expression of an exogenous nucleic acid in foveal cones of a mammal (e.g., a human or a non-human primate) that is greater than (e.g., at least 10 percent greater than, at least 25 percent greater than, at least 50 percent greater than, at least 75 percent greater than, or at least 100 percent greater than) the level of mRNA expression of an exogenous nucleic acid driven by a comparable AAV vector having an AAV capsid polypeptide that consists of the amino acid sequence of SEQ ID NO:1 (e.g., a wild-type AAV2 vector) in foveal cones of a control mammal (e.g., a control human or a control non-human primate). In some cases, an AAV vector (e.g., an AAV2 vector) provided herein having an AAV capsid polypeptide that includes an amino acid sequence set forth in Table 1 (or a variant thereof) or Formula A can be used in place of the 7m8 AAV2 vector (Dalkara et al., Sci. Transl. Med., 5 (189): 189ra76 (2013) and Bennett et al., J. Struct. Biol., 209 (2): 107433 (2020)) or in place of the K912 AAV2 vector (Öztürk et al., eLife, 10: e64175 (2021)) to deliver nucleic acid to foveal cones.

In another aspect, this document provides AAV vectors (e.g., AAV2 vectors) containing a capsid polypeptide that includes an amino acid sequence set forth in Table 1 (or a variant thereof) or Formula A. The AAV vectors (e.g., AAV2 vectors) described herein containing a capsid polypeptide that includes an amino acid sequence set forth in Table 1 (or a variant thereof) or Formula A can have the ability to infect retinal cells (e.g., retinal ganglion cells, photoreceptor cells, and bipolar cells) in vivo and deliver exogenous nucleic acid to the infected retinal cells such that the infected retinal cells express the exogenous nucleic acid at high levels. This document also provides methods and materials for making and using AAV vectors (e.g., AAV2 vectors) having the ability to deliver nucleic acid to retinal cells and drive high expression levels of nucleic acid within retinal cells.

As described herein, an AAV vector (e.g., an AAV2 vector) provided herein having an AAV capsid polypeptide that includes an amino acid sequence set forth in Table 1 (or a variant thereof) or Formula A can have the ability to infect retinal cells (e.g., retinal ganglion cells, photoreceptor cells, and bipolar cells) in vivo and deliver exogenous nucleic acid to the infected retinal cells such that the infected retinal cells express the exogenous nucleic acid at high levels. In some cases, an AAV vector (e.g., an AAV2 vector) provided herein can have the ability to infect and drive mRNA expression of an exogenous nucleic acid in at least 2 percent (e.g., at least 2.5 percent, at least 5 percent, at least 7.5 percent, at least 10 percent, or at least 25 percent) of retinal cells (e.g., retinal ganglion cells, photoreceptor cells, and bipolar cells) in an eye of a mammal (e.g., a human or a non-human primate). In some cases, an AAV vector (e.g., an AAV2 vector) provided herein can have the ability to drive a level of mRNA expression of an exogenous nucleic acid in retinal cells (e.g., retinal ganglion cells, photoreceptor cells, and bipolar cells) of a mammal (e.g., a human or a non-human primate) that is greater than (e.g., at least 2 percent greater than, at least 2.5 percent greater than, at least 5 percent greater than, at least 7.5 percent greater than, at least 10 percent greater than, at least 25 percent greater than, at least 50 percent greater than, at least 75 percent greater than, or at least 100 percent greater than) the level of mRNA expression of an exogenous nucleic acid driven by a comparable AAV vector having an AAV capsid polypeptide that consists of the amino acid sequence of SEQ ID NO: 1 (e.g., a wild-type AAV2 vector) in retinal cells of a control mammal (e.g., a control human or a control non-human primate). In some cases, an AAV vector (e.g., an AAV2 vector) provided herein having an AAV capsid polypeptide that includes an amino acid sequence set forth in Table I (or a variant thereof) or Formula A can be used in place of the 7m8 AAV2 vector (Dalkara et al., Sci. Transl. Med., 5(189): 189ra76 (2013) and Bennett et al., J. Struct. Biol., 209 (2): 107433 (2020)) or in place of the K912 AAV2 vector (Öztürk et al., eLife, 10: e64175 (2021)) to deliver nucleic acid to retinal cells (e.g., retinal ganglion cells, photoreceptor cells, and bipolar cells).

In another aspect, this document provides AAV vectors (e.g., AAV2 vectors) containing a capsid polypeptide that includes an amino acid sequence set forth in Table 1 (or a variant thereof) or Formula A. The AAV vectors (e.g., AAV2 vectors) described herein containing a capsid polypeptide that includes an amino acid sequence set forth in Table 1 (or a variant thereof) or Formula A can have the ability to infect retinal cells (e.g., retinal ganglion cells, photoreceptor cells, and bipolar cells) across retinal regions (e.g., across at least two retinal regions) in vivo and deliver exogenous nucleic acid to the infected retinal cells such that the infected retinal cells express the exogenous nucleic acid. This document also provides methods and materials for making and using AAV vectors (e.g., AAV2 vectors) having the ability to deliver nucleic acid to retinal cells across retinal regions and drive expression of delivered nucleic acid within the retinal cells. For example, the AAV vectors (e.g., AAV2 vectors) described herein can deliver nucleic acid to at least about 2 percent (e.g., at least about 2.5 percent, at least about 5 percent, at least about 7.5 percent, at least about 10 percent, or at least about 25 percent) of the retinal cells in the fovea region, at least about 2 percent (e.g., at least about 2.5 percent, at least about 5 percent, at least about 7.5 percent, at least about 10 percent, or at least about 25 percent) of the retinal cells in the parafovea region, at least about 2 percent (e.g., at least about 2.5 percent, at least about 5 percent, at least about 7.5 percent, at least about 10 percent, or at least about 25 percent) of the retinal cells in the vascular arcade region, and/or at least about 2 percent (e.g., at least about 2.5 percent, at least about 5 percent, at least about 7.5 percent, at least about 10 percent, or at least about 25 percent) of the retinal cells in the periphery region.

As described herein, an AAV vector (e.g., an AAV2 vector) provided herein having an AAV capsid polypeptide that includes an amino acid sequence set forth in Table 1 (or a variant thereof) or Formula A can have the ability to infect retinal cells (e.g., retinal ganglion cells, photoreceptor cells, and bipolar cells) across retinal regions (e.g., across at least two retinal regions) in vivo and deliver exogenous nucleic acid to the infected retinal cells such that the infected retinal cells express the exogenous nucleic acid. In some cases, an AAV vector (e.g., an AAV2 vector) provided herein can have the ability to infect and drive mRNA expression of an exogenous nucleic acid in at least about 2 percent (e.g., at least about 2.5 percent, at least about 5 percent, at least about 7.5 percent, at least about 10 percent, or at least about 25 percent) of the retinal cells in the fovea region, at least about 2 percent (e.g., at least about 2.5 percent, at least about 5 percent, at least about 7.5 percent, at least about 10 percent, or at least about 25 percent) of the retinal cells in the parafovea region, at least about 2 percent (e.g., at least about 2.5 percent, at least about 5 percent, at least about 7.5 percent, at least about 10 percent, or at least about 25 percent) of the retinal cells in the vascular arcade region, and/or at least about 2 percent (e.g., at least about 2.5 percent, at least about 5 percent, at least about 7.5 percent, at least about 10 percent, or at least about 25 percent) of the retinal cells in the periphery region of an eye of a mammal (e.g., a human or a non-human primate). In some cases, an AAV vector (e.g., an AAV2 vector) provided herein can have the ability to drive a level of mRNA expression of an exogenous nucleic acid in retinal cells of the fovea region, the parafovea region, the vascular arcade region, and/or the periphery region of an eye of a mammal (e.g., a human or a non-human primate) that is greater than the level of mRNA expression of an exogenous nucleic acid driven by a comparable AAV vector having an AAV capsid polypeptide that consists of the amino acid sequence of SEQ ID NO: 1 (e.g., a wild-type AAV2 vector) in retinal cells of those regions in a control mammal (e.g., a control human or a control non-human primate). In some cases, an AAV vector (e.g., an AAV2 vector) provided herein having an AAV capsid polypeptide that includes an amino acid sequence set forth in Table 1 (or a variant thereof) or Formula A can be used in place of the 7m8 AAV2 vector (Dalkara et al., Sci. Transl. Med., 5 (189): 189ra76 (2013) and Bennett et al., J. Struct. Biol., 209 (2): 107433 (2020)) or in place of the K912 AAV2 vector (Öztürk et al., eLife, 10: e64175 (2021)) to deliver nucleic acid to retinal cells (e.g., retinal ganglion cells, photoreceptor cells, and bipolar cells).

In another aspect, this document provides AAV vectors (e.g., AAV2 vectors) containing a capsid polypeptide that includes an amino acid sequence set forth in Table 1 (or a variant thereof) or Formula A. The AAV vectors (e.g., AAV2 vectors) described herein containing a capsid polypeptide that includes an amino acid sequence set forth in Table 1 (or a variant thereof) or Formula A can have the ability to infect retinal cells of the parafovea region of the eye in vivo and deliver exogenous nucleic acid to the infected retinal cells of the parafovea region such that the infected retinal cells express the exogenous nucleic acid. This document also provides methods and materials for making and using AAV vectors (e.g., AAV2 vectors) having the ability to deliver nucleic acid to retinal cells of the parafovea region of the eye.

As described herein, an AAV vector (e.g., an AAV2 vector) provided herein having an AAV capsid polypeptide that includes an amino acid sequence set forth in Table 1 (or a variant thereof) or Formula A can have the ability to infect retinal cells of the parafovea region of the eye in vivo and deliver exogenous nucleic acid to the infected retinal cells such that the infected retinal cells express the exogenous nucleic acid. In some cases, an AAV vector (e.g., an AAV2 vector) provided herein can have the ability to infect and drive mRNA expression of an exogenous nucleic acid in at least about 2 percent (e.g., at least about 2.5 percent, at least about 5 percent, at least about 7.5 percent, at least about 10 percent, or at least about 25 percent) of the retinal cells present in the parafovea region of an eye of a mammal (e.g., a human or a non-human primate). In some cases, an AAV vector (e.g., an AAV2 vector) provided herein can have the ability to drive a level of mRNA expression of an exogenous nucleic acid in retinal cells of the parafovea region of the eye of a mammal (e.g., a human or a non-human primate) that is greater than (e.g., at least 10 percent greater than, at least 25 percent greater than, at least 50 percent greater than, at least 75 percent greater than, or at least 100 percent greater than) the level of mRNA expression of an exogenous nucleic acid driven by a comparable AAV vector having an AAV capsid polypeptide that consists of the amino acid sequence of SEQ ID NO: 1 (e.g., a wild-type AAV2 vector) in retinal cells of the parafovea region of the eye of a control mammal (e.g., a control human or a control non-human primate). In some cases, an AAV vector (e.g., an AAV2 vector) provided herein having an AAV capsid polypeptide that includes an amino acid sequence set forth in Table 1 (or a variant thereof) or Formula A can be used in place of the 7m8 AAV2 vector (Dalkara et al., Sci. Transl. Med., 5 (189): 189ra76 (2013) and Bennett et al., J. Struct. Biol., 209 (2): 107433 (2020)) or in place of the K912 AAV2 vector (Öztürk et al., eLife, 10: e64175 (2021)) to deliver nucleic acid to retinal cells (e.g., retinal ganglion cells, photoreceptor cells, and bipolar cells) of the parafovea region of the eye.

In another aspect, this document provides AAV vectors (e.g., AAV2 vectors) containing a capsid polypeptide that includes an amino acid sequence set forth in Table 1 (or a variant thereof) or Formula A. The AAV vectors (e.g., AAV2 vectors) described herein containing a capsid polypeptide that includes an amino acid sequence set forth in Table 1 (or a variant thereof) or Formula A can have the ability to infect two or more (e.g., two or more, three or more, four or more, five or more, six or more, or seven or more) different retinal cell types within an eye in vivo and deliver exogenous nucleic acid to the infected retinal cells such that the infected retinal cells express the exogenous nucleic acid. This document also provides methods and materials for making and using AAV vectors (e.g., AAV2 vectors) having the ability to deliver nucleic acid to two or more (e.g., two or more, three or more, four or more, five or more, six or more, or seven or more) different retinal cell types within an eye and drive expression of delivered nucleic acid within those retinal cells. For example, the AAV vectors (e.g., AAV2 vectors) described herein can deliver nucleic acid to two, three, four, five, six, or seven of the following retinal cell types of an eye: retinal ganglion cells, amacrine cells, horizontal cells, bipolar cells, Muller glia cells, photoreceptor cells, and retinal pigment epithelial (RPE) cells. In some cases, an AAV vector (e.g., an AAV2 vector) described herein can deliver nucleic acid to at least some (e.g., at least 2 percent, at least 2.5 percent, at least 5 percent, at least 10 percent, or at least 25 percent) of the retinal ganglion cells, amacrine cells, horizontal cells, bipolar cells, Muller glia cells, photoreceptor cells, and RPE cells of an eye of a mammal (e.g., a human or a non-human primate) following an intravitreal administration.

As described herein, an AAV vector (e.g., an AAV2 vector) provided herein having an AAV capsid polypeptide that includes an amino acid sequence set forth in Table 1 (or a variant thereof) or Formula A can have the ability to infect two or more (e.g., two or more, three or more, four or more, five or more, six or more, or seven or more) different retinal cell types within an eye in vivo and deliver exogenous nucleic acid to the infected retinal cells such that the infected retinal cells express the exogenous nucleic acid. In some cases, an AAV vector (e.g., an AAV2 vector) provided herein can have the ability to infect and drive mRNA expression of an exogenous nucleic acid in at least about 2 percent (e.g., at least about 2.5 percent, at least about 5 percent, at least about 7.5 percent, at least about 10 percent, or at least about 25 percent) of the retinal ganglion cells, at least about 2 percent (e.g., at least about 2.5 percent, at least about 5 percent, at least about 7.5 percent, at least about 10 percent, or at least about 25 percent) of the amacrine cells, at least about 2 percent (e.g., at least about 2.5 percent, at least about 5 percent, at least about 7.5 percent, at least about 10 percent, or at least about 25 percent) of the horizontal cells, at least about 2 percent (e.g., at least about 2.5 percent, at least about 5 percent, at least about 7.5 percent, at least about 10 percent, or at least about 25 percent) of the bipolar cells, at least about 2 percent (e.g., at least about 2.5 percent, at least about 5 percent, at least about 7.5 percent, at least about 10 percent, or at least about 25 percent) of the Muller glia cells, at least about 2 percent (e.g., at least about 2.5 percent, at least about 5 percent, at least about 7.5 percent, at least about 10 percent, or at least about 25 percent) of the photoreceptor cells, and/or at least about 2 percent (e.g., at least about 2.5 percent, at least about 5 percent, at least about 7.5 percent, at least about 10 percent, or at least about 25 percent) of the RPE cells of an eye of a mammal (e.g., a human or a non-human primate) following, for example, an intravitreal administration. In some cases, an AAV vector (e.g., an AAV2 vector) provided herein can have the ability to drive a level of mRNA expression of an exogenous nucleic acid in retinal ganglion cells, amacrine cells, horizontal cells, bipolar cells, Muller glia cells, photoreceptor cells, and/or RPE cells of an eye of a mammal (e.g., a human or a non-human primate) that is greater than (e.g., at least 10 percent greater than, at least 25 percent greater than, at least 50 percent greater than, at least 75 percent greater than, or at least 100 percent greater than) the level of mRNA expression of an exogenous nucleic acid driven by a comparable AAV vector having an AAV capsid polypeptide that consists of the amino acid sequence of SEQ ID NO: 1 (e.g., a wild-type AAV2 vector) in those retinal cells in a control mammal (e.g., a control human or a control non-human primate). In some cases, an AAV vector (e.g., an AAV2 vector) provided herein having an AAV capsid polypeptide that includes an amino acid sequence set forth in Table 1 (or a variant thereof) or Formula A can be used in place of the 7m8 AAV2 vector (Dalkara et al., Sci. Transl. Med., 5 (189): 189ra76 (2013) and Bennett et al., J. Struct. Biol., 209 (2): 107433 (2020)) or in place of the K912 AAV2 vector (Öztürk et al., eLife, 10: e64175 (2021)) to deliver nucleic acid to two or more (e.g., two or more, three or more, four or more, five or more, six or more, or seven or more) different retinal cell types within an eye of a mammal (e.g., a human or a non-human primate).

In another aspect, this document provides AAV vectors (e.g., AAV2 vectors) containing a capsid polypeptide that includes an amino acid sequence set forth in Table 1 (or a variant thereof) or Formula A. The AAV vectors (e.g., AAV2 vectors) described herein containing a capsid polypeptide that includes an amino acid sequence set forth in Table 1 (or a variant thereof) or Formula A can have the ability to infect RPE cells in vivo and deliver exogenous nucleic acid to the infected RPE cells such that the infected RPE cells express the exogenous nucleic acid. This document also provides methods and materials for making and using AAV vectors (e.g., AAV2 vectors) having the ability to deliver nucleic acid to RPE cells and drive expression of delivered nucleic acid within the RPE cells. For example, the AAV vectors (e.g., AAV2 vectors) described herein can deliver nucleic acid to at least about 2 percent (e.g., at least about 2.5 percent, at least about 5 percent, at least about 7.5 percent, at least about 10 percent, or at least about 25 percent) of the RPE cells of an eye of a mammal after, for example, an intravitreal administration.

As described herein, an AAV vector (e.g., an AAV2 vector) provided herein having an AAV capsid polypeptide that includes an amino acid sequence set forth in Table 1 (or a variant thereof) or Formula A can have the ability to infect RPE cells in vivo and deliver exogenous nucleic acid to the infected RPE cells such that the infected RPE cells express the exogenous nucleic acid. In some cases, an AAV vector (e.g., an AAV2 vector) provided herein can have the ability to infect and drive mRNA expression of an exogenous nucleic acid in at least about 2 percent (e.g., at least about 2.5 percent, at least about 5 percent, at least about 7.5 percent, at least about 10 percent, or at least about 25 percent) of the RPE cells of an eye of a mammal (e.g., a human or a non-human primate). In some cases, an AAV vector (e.g., an AAV2 vector) provided herein can have the ability to drive a level of mRNA expression of an exogenous nucleic acid in RPE cells of an eye of a mammal (e.g., a human or a non-human primate) that is greater than (e.g., at least 10 percent greater than, at least 25 percent greater than, at least 50 percent greater than, at least 75 percent greater than, or at least 100 percent greater than) the level of mRNA expression of an exogenous nucleic acid driven by a comparable AAV vector having an AAV capsid polypeptide that consists of the amino acid sequence of SEQ ID NO: 1 (e.g., a wild type AAV2 vector) in RPE cells in a control mammal (e.g., a control human or a control non-human primate). In some cases, an AAV vector (e.g., an AAV2 vector) provided herein having an AAV capsid polypeptide that includes an amino acid sequence set forth in Table 1 (or a variant thereof) or Formula A can be used in place of the 7m8 AAV2 vector (Dalkara et al., Sci. Transl. Med., 5 (189): 189ra76 (2013) and Bennett et al., J. Struct. Biol., 209 (2): 107433 (2020)) or in place of the K912 AAV2 vector (Öztürk et al., eLife, 10: e64175 (2021)) to deliver nucleic acid to RPE cells.

In another aspect, this document provides AAV vectors (e.g., AAV2 vectors) containing a capsid polypeptide that includes an amino acid sequence set forth in Table 1 (or a variant thereof) or Formula A. The AAV vectors (e.g., AAV2 vectors) described herein containing a capsid polypeptide that includes an amino acid sequence set forth in Table 1 (or a variant thereof) or Formula A can have the ability to infect photoreceptor cells of the retina in vivo and deliver exogenous nucleic acid to the infected photoreceptor cells such that the infected photoreceptor cells express the exogenous nucleic acid. This document also provides methods and materials for making and using AAV vectors (e.g., AAV2 vectors) having the ability to deliver nucleic acid to photoreceptor cells of the retina.

As described herein, an AAV vector (e.g., an AAV2 vector) provided herein having an AAV capsid polypeptide that includes an amino acid sequence set forth in Table I (or a variant thereof) or Formula A can have the ability to infect photoreceptor cells of the retina in vivo and deliver exogenous nucleic acid to the infected photoreceptor cells such that the infected photoreceptor cells express the exogenous nucleic acid. In some cases, an AAV vector (e.g., an AAV2 vector) provided herein can have the ability to infect and drive mRNA expression of an exogenous nucleic acid in at least about 2 percent (e.g., at least about 2.5 percent, at least about 5 percent, at least about 7.5 percent, at least about 10 percent, or at least about 25 percent) of photoreceptor cells of an eye of a mammal (e.g., a human or a non-human primate). In some cases, an AAV vector (e.g., an AAV2 vector) provided herein can have the ability to drive a level of mRNA expression of an exogenous nucleic acid in photoreceptor cells of the retina of a mammal (e.g., a human or a non-human primate) that is greater than (e.g., at least 10 percent greater than, at least 25 percent greater than, at least 50 percent greater than, at least 75 percent greater than, or at least 100 percent greater than) the level of mRNA expression of an exogenous nucleic acid driven by a comparable AAV vector having an AAV capsid polypeptide that consists of the amino acid sequence of SEQ ID NO: 1 (e.g., a wild-type AAV2 vector) in photoreceptor cells of the retina of a control mammal (e.g., a control human or a control non-human primate). In some cases, an AAV vector (e.g., an AAV2 vector) provided herein having an AAV capsid polypeptide that includes an amino acid sequence set forth in Table 1 (or a variant thereof) or Formula A can be used in place of the 7m8 AAV2 vector (Dalkara et al., Sci. Transl. Med., 5 (189): 189ra76 (2013) and Bennett et al., J. Struct. Biol., 209 (2): 107433 (2020)) or in place of the K912 AAV2 vector (Öztürk et al., eLife, 10: e64175 (2021)) to deliver nucleic acid to photoreceptor cells of the retina.

In another aspect, this document provides AAV vectors (e.g., AAV2 vectors) containing a capsid polypeptide that includes an amino acid sequence set forth in Table 1 (or a variant thereof) or Formula A. The AAV vectors (e.g., AAV2 vectors) described herein containing a capsid polypeptide that includes an amino acid sequence set forth in Table 1 (or a variant thereof) or Formula A can have the ability to infect retinal ganglion cells in vivo and deliver exogenous nucleic acid to the infected retinal ganglion cells such that the infected retinal ganglion cells express the exogenous nucleic acid. This document also provides methods and materials for making and using AAV vectors (e.g., AAV2 vectors) having the ability to deliver nucleic acid to retinal ganglion cells.

As described herein, an AAV vector (e.g., an AAV2 vector) provided herein having an AAV capsid polypeptide that includes an amino acid sequence set forth in Table 1 (or a variant thereof) or Formula A can have the ability to infect retinal ganglion cells in vivo and deliver exogenous nucleic acid to the infected retinal ganglion cells such that the infected retinal ganglion cells express the exogenous nucleic acid. In some cases, an AAV vector (e.g., an AAV2 vector) provided herein can have the ability to infect and drive mRNA expression of an exogenous nucleic acid in at least about 2 percent (e.g., at least about 2.5 percent, at least about 5 percent, at least about 7.5 percent, at least about 10 percent, or at least about 25 percent) of retinal ganglion cells of an eye of a mammal (e.g., a human or a non-human primate). In some cases, an AAV vector (e.g., an AAV2 vector) provided herein can have the ability to drive a level of mRNA expression of an exogenous nucleic acid in retinal ganglion cells of a mammal (e.g., a human or a non-human primate) that is greater than (e.g., at least 10 percent greater than, at least 25 percent greater than, at least 50 percent greater than, at least 75 percent greater than, or at least 100 percent greater than) the level of mRNA expression of an exogenous nucleic acid driven by a comparable AAV vector having an AAV capsid polypeptide that consists of the amino acid sequence of SEQ ID NO:1 (e.g., a wild-type AAV2 vector) in retinal ganglion cells of an eye of a control mammal (e.g., a control human or a control non-human primate). In some cases, an AAV vector (e.g., an AAV2 vector) provided herein having an AAV capsid polypeptide that includes an amino acid sequence set forth in Table 1 (or a variant thereof) or Formula A can be used in place of the 7m8 AAV2 vector (Dalkara et al., Sci. Transl. Med., 5 (189): 189ra76 (2013) and Bennett et al., J. Struct. Biol., 209 (2): 107433 (2020)) or in place of the K912 AAV2 vector (Öztürk et al., eLife, 10: e64175 (2021)) to deliver nucleic acid to photoreceptor cells of the retina.

In another aspect, this document provides AAV vectors (e.g., AAV2 vectors) containing a capsid polypeptide that includes an amino acid sequence set forth in Table 1 (or a variant thereof) or Formula A. The AAV vectors (e.g., AAV2 vectors) described herein containing a capsid polypeptide that includes an amino acid sequence set forth in Table 1 (or a variant thereof) or Formula A can have the ability to infect bipolar cells of the retina in vivo and deliver exogenous nucleic acid to the infected bipolar cells such that the infected retinal bipolar cells express the exogenous nucleic acid. This document also provides methods and materials for making and using AAV vectors (e.g., AAV2 vectors) having the ability to deliver nucleic acid to bipolar cells of the retina.

As described herein, an AAV vector (e.g., an AAV2 vector) provided herein having an AAV capsid polypeptide that includes an amino acid sequence set forth in Table 1 (or a variant thereof) or Formula A can have the ability to infect bipolar cells of the retina in vivo and deliver exogenous nucleic acid to the infected bipolar cells such that the infected bipolar cells express the exogenous nucleic acid. In some cases, an AAV vector (e.g., an AAV2 vector) provided herein can have the ability to infect and drive mRNA expression of an exogenous nucleic acid in at least about 2 percent (e.g., at least about 2.5 percent, at least about 5 percent, at least about 7.5 percent, at least about 10 percent, or at least about 25 percent) of bipolar cells of the retina of a mammal (e.g., a human or a non-human primate). In some cases, an AAV vector (e.g., an AAV2 vector) provided herein can have the ability to drive a level of mRNA expression of an exogenous nucleic acid in bipolar cells of the retina of a mammal (e.g., a human or a non-human primate) that is greater than (e.g., at least 10 percent greater than, at least 25 percent greater than, at least 50 percent greater than, at least 75 percent greater than, or at least 100 percent greater than) the level of mRNA expression of an exogenous nucleic acid driven by a comparable AAV vector having an AAV capsid polypeptide that consists of the amino acid sequence of SEQ ID NO:1 (e.g., a wild-type AAV2 vector) in bipolar cells of the retina of a control mammal (e.g., a control human or a control non-human primate). In some cases, an AAV vector (e.g., an AAV2 vector) provided herein having an AAV capsid polypeptide that includes an amino acid sequence set forth in Table 1 (or a variant thereof) or Formula A can be used in place of the 7m8 AAV2 vector (Dalkara et al., Sci. Transl. Med., 5 (189): 189ra76 (2013) and Bennett et al., J. Struct. Biol., 209 (2): 107433 (2020)) or in place of the K912 AAV2 vector (Öztürk et al., eLife, 10: e64175 (2021)) to deliver nucleic acid to photoreceptor cells of the retina.

In another aspect, this document provides AAV vectors (e.g., AAV2 vectors) containing a capsid polypeptide that includes an amino acid sequence set forth in Table 1 (or a variant thereof) or Formula A. The AAV vectors (e.g., AAV2 vectors) described herein containing a capsid polypeptide that includes an amino acid sequence set forth in Table 1 (or a variant thereof) or Formula A can have the ability to infect OFF-retinal ganglion cells in vivo and deliver exogenous nucleic acid to the infected OFF-retinal ganglion cells such that the infected OFF-retinal ganglion cells express the exogenous nucleic acid. This document also provides methods and materials for making and using AAV vectors (e.g., AAV2 vectors) having the ability to deliver nucleic acid to OFF-retinal ganglion cells.

As described herein, an AAV vector (e.g., an AAV2 vector) provided herein having an AAV capsid polypeptide that includes an amino acid sequence set forth in Table I (or a variant thereof) or Formula A can have the ability to infect OFF-retinal ganglion cells in vivo and deliver exogenous nucleic acid to the infected OFF-retinal ganglion cells such that the infected OFF-retinal ganglion cells express the exogenous nucleic acid. In some cases, an AAV vector (e.g., an AAV2 vector) provided herein can have the ability to infect and drive mRNA expression of an exogenous nucleic acid in at least about 2 percent (e.g., at least about 2.5 percent, at least about 5 percent, at least about 7.5 percent, at least about 10 percent, or at least about 25 percent) of OFF-retinal ganglion cells of a mammal (e.g., a human or a non-human primate). In some cases, an AAV vector (e.g., an AAV2 vector) provided herein can have the ability to drive a level of mRNA expression of an exogenous nucleic acid in OFF-retinal ganglion cells of a mammal (e.g., a human or a non-human primate) that is greater than (e.g., at least 10 percent greater than, at least 25 percent greater than, at least 50 percent greater than, at least 75 percent greater than, or at least 100 percent greater than) the level of mRNA expression of an exogenous nucleic acid driven by a comparable AAV vector having an AAV capsid polypeptide that consists of the amino acid sequence of SEQ ID NO: 1 (e.g., a wild-type AAV2 vector) in OFF-retinal ganglion cells of a control mammal (e.g., a control human or a control non-human primate). In some cases, an AAV vector (e.g., an AAV2 vector) provided herein having an AAV capsid polypeptide that includes an amino acid sequence set forth in Table 1 (or a variant thereof) or Formula A can be used in place of the 7m8 AAV2 vector (Dalkara et al., Sci. Transl. Med., 5 (189): 189ra76 (2013) and Bennett et al., J. Struct. Biol., 209 (2): 107433 (2020)) or in place of the K912 AAV2 vector (Öztürk et al., eLife, 10: e64175 (2021)) to deliver nucleic acid to photoreceptor cells of the retina.

In another aspect, this document provides AAV vectors (e.g., AAV2 vectors) containing a capsid polypeptide that includes an amino acid sequence set forth in Table 1 (or a variant thereof) or Formula A. The AAV vectors (e.g., AAV2 vectors) described herein containing a capsid polypeptide that includes an amino acid sequence set forth in Table 1 (or a variant thereof) or Formula A can have increased packaging efficiency, the ability to infect cells (e.g., retinal cells) in vivo or in vitro, and the ability to deliver exogenous nucleic acid to the infected cells such that the infected cells express the exogenous nucleic acid. This document also provides methods and materials for making and using AAV vectors (e.g., AAV2 vectors) having increased packaging efficiency, the ability to deliver nucleic acid to cells (e.g., retinal cells) in vivo or in vitro, and the ability to drive expression of delivered nucleic acid within the cells. For example, the AAV vectors (e.g., AAV2 vectors) described herein can have a packaging efficiency greater than that of a comparable AAV vector having an AAV capsid polypeptide that consists of the amino acid sequence of SEQ ID NO: 1 (e.g., a wild type AAV2 vector).

As described herein, an AAV vector (e.g., an AAV2 vector) provided herein having an AAV capsid polypeptide that includes an amino acid sequence set forth in Table 1 (or a variant thereof) or Formula A can have increased packaging efficiency (e.g., package efficiency greater than that of a comparable AAV vector having an AAV capsid polypeptide that consists of the amino acid sequence of SEQ ID NO: 1 (e.g., a wild type AAV2 vector)), the ability to infect cells (e.g., retinal cells) in vivo or in vitro, and the ability to drive exogenous nucleic acid to the infected cells such that the infected cells express the exogenous nucleic acid. In some cases, an AAV vector (e.g., an AAV2 vector) provided herein can have a packaging efficiency that is greater than (e.g., at least 10 percent greater than, at least 25 percent greater than, at least 50 percent greater than, at least 75 percent greater than, or at least 100 percent greater than) the packaging efficiency of a comparable AAV vector having an AAV capsid polypeptide that consists of the amino acid sequence of SEQ ID NO:1 (e.g., a wild-type AAV2 vector). In some cases, an AAV vector (e.g., an AAV2 vector) provided herein having an AAV capsid polypeptide that includes an amino acid sequence set forth in Table 1 (or a variant thereof) or Formula A can have a packaging efficiency greater than that of the 7m8 AAV2 vector (Dalkara et al., Sci. Transl. Med., 5 (189): 189ra76 (2013) and Bennett et al., J. Struct. Biol., 209 (2): 107433 (2020)) or the K912 AAV2 vector (Öztürk et al., eLife, 10: e64175 (2021)).

In general, one aspect of this document features an adeno-associated virus (AAV) vector (e.g., an AAV2 vector) comprising an AAV capsid polypeptide, wherein the capsid polypeptide comprises the amino acid sequence of any one of SEQ ID NOs: 2-5. The capsid polypeptide can comprise the amino acid sequence of SEQ ID NO: 1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs: 2-5 is located between amino acid positions 587 and 588 of SEQ ID NO: 1 (or the appropriate amino acid positions of the alternative sequence). The capsid polypeptide can comprise the amino acid sequence of SEQ ID NO:1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO: 1) except that the amino acid sequence of SEQ ID NO:5 is located between amino acid positions 587 and 588 of SEQ ID NO: 1 (or the appropriate amino acid positions of the alternative sequence). The capsid polypeptide can comprise the amino acid sequence of SEQ ID NO: 1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO: 1 (or the appropriate amino acid positions of the alternative sequence) are replaced with the amino acid sequence of any one of SEQ ID NOs: 2-5. The capsid polypeptide can comprise the amino acid sequence of SEQ ID NO: 1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO: 1 (or the appropriate amino acid positions of the alternative sequence) are replaced with the amino acid sequence of SEQ ID NO:2. The vector can be an AAV2 vector. The vector can infect greater than 2.5 percent of retinal cells when a titer of at least 1×10⁷ of the vector is administered intravitreally to an eye of a human (or a non-human primate). The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in retinal cells than the level of expression from a comparable AAV vector comprising a capsid polypeptide consisting of the amino acid sequence set forth in SEQ ID NO:1.

In another aspect, this document features an AAV capsid polypeptide comprising the amino acid sequence of any one of SEQ ID NOs: 2-5. The polypeptide can comprise the amino acid sequence of SEQ ID NO: 1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs: 2-5 is located between amino acid positions 587 and 588 of SEQ ID NO:1 (or the appropriate amino acid positions of the alternative sequence). The polypeptide can comprise the amino acid sequence of SEQ ID NO: 1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO: 1) except that the amino acid sequence of SEQ ID NO:5 is located between amino acid positions 587 and 588 of SEQ ID NO: 1 (or the appropriate amino acid positions of the alternative sequence). The polypeptide can comprise the amino acid sequence of SEQ ID NO: 1 (or an alternative sequence that is the amino acid sequence set forth in SEQ ID NO:10 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO: 1 (or the appropriate amino acid positions of the alternative sequence) are replaced with the amino acid sequence of any one of SEQ ID NOs: 2-5. The polypeptide can comprise the amino acid sequence of SEQ ID NO: 1 (or an alternative sequence that is the amino acid sequence set forth in SEQ ID NO:10 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO: 1 (or the appropriate amino acid positions of the alternative sequence, e.g., SEQ ID NO:10) are replaced with the amino acid sequence of SEQ ID NO:2. In some cases, the capsid polypeptide can comprise or consist of the amino acid sequence of any of SEQ ID NOs: 11-26. An AAV vector comprising the polypeptide can infect greater than 2.5 percent of retinal cells when a titer of at least 1×10⁷ of the vector is administered intravitreally to an eye of a human (or a non-human primate). An AAV vector comprising the polypeptide can express more nucleic acid in retinal cells than the level of expression from a comparable AAV vector comprising a capsid polypeptide consisting of the amino acid sequence set forth in SEQ ID NO:1.

In another aspect, this document features a nucleic acid molecule encoding an adeno-associated virus (AAV) vector comprising an AAV capsid polypeptide, wherein the capsid polypeptide comprises the amino acid sequence of any one of SEQ ID NOs: 2-5. The capsid polypeptide can comprise the amino acid sequence of SEQ ID NO: 1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO: 1) except that the amino acid sequence of any one of SEQ ID NOs: 2-5 is located between amino acid positions 587 and 588 of SEQ ID NO: 1 (or the appropriate amino acid positions of the alternative sequence, e.g., SEQ ID NO: 10). In some cases, the capsid polypeptide can comprise or consist of the amino acid sequence of any of SEQ ID NOs: 11-26. The capsid polypeptide can comprise the amino acid sequence of SEQ ID NO: 1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO: 1) except that the amino acid sequence of SEQ ID NO:5 is located between amino acid positions 587 and 588 of SEQ ID NO:1 (or the appropriate amino acid positions of the alternative sequence). The capsid polypeptide can comprise the amino acid sequence of SEQ ID NO: 1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO: 1 (or the appropriate amino acid positions of the alternative sequence) are replaced with the amino acid sequence of any one of SEQ ID NOs: 2-5. In some cases, the capsid polypeptide can comprise or consist of the amino acid sequence of any of SEQ ID NOs: 27-42. The capsid polypeptide can comprise the amino acid sequence of SEQ ID NO: 1 (or an alternative sequence that is the amino acid sequence set forth in SEQ ID NO:10 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 (or the appropriate amino acid positions of the alternative sequence) are replaced with the amino acid sequence of SEQ ID NO:2. The vector can be an AAV2 vector. The vector can infect greater than 2.5 percent of retinal cells when a titer of at least 1×10⁷ of the vector is administered intravitreally to an eye of a human (or a non-human primate). The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in retinal cells than the level of expression from a comparable AAV vector comprising a capsid polypeptide consisting of the amino acid sequence set forth in SEQ ID NO: 1. The nucleic acid molecule can be DNA.

In another aspect, this document features a nucleic acid molecule encoding an AAV capsid polypeptide comprising the amino acid sequence of any one of SEQ ID NOs: 2-5. The polypeptide can comprise the amino acid sequence of SEQ ID NO:1 (or an alternative sequence that is the amino acid sequence set forth in SEQ ID NO:10 or that is an amino acid sequence at least 95%, at least 96%, at least 97%, at least 98%, or at least 99% identical to the amino acid sequence set forth in SEQ ID NO: 1) except that the amino acid sequence of any one of SEQ ID NOs: 2-5 is located between amino acid positions 587 and 588 of SEQ ID NO: 1 (or the appropriate amino acid positions of the alternative sequence). The polypeptide can comprise the amino acid sequence of SEQ ID NO: 1 (or an alternative sequence that is the amino acid sequence set forth in SEQ ID NO:10 or that is an amino acid sequence at least 95%, at least 96%, at least 97%, at least 98%, or at least 99% identical to the amino acid sequence set forth in SEQ ID NO:1) except that the amino acid sequence of SEQ ID NO:5 is located between amino acid positions 587 and 588 of SEQ ID NO: 1 (or the appropriate amino acid positions of the alternative sequence). The polypeptide can comprise the amino acid sequence of SEQ ID NO: 1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO:1 (or the appropriate amino acid positions of the alternative sequence) are replaced with the amino acid sequence of any one of SEQ ID NOs: 2-5. The capsid polypeptide can comprise or consist of the amino acid sequence of any of SEQ ID NOs: 11-42. The polypeptide can comprise the amino acid sequence of SEQ ID NO: 1 (or an alternative sequence that is the amino acid sequence set forth in SEQ ID NO:10 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO: 1 (or the appropriate amino acid positions of the alternative sequence) are replaced with the amino acid sequence of SEQ ID NO:2. An AAV vector comprising the polypeptide can infect greater than 2.5 percent of retinal cells when a titer of at least 1×10⁷ of the vector is administered intravitreally to an eye of a human (or a non-human primate). An AAV vector comprising the polypeptide can express more nucleic acid in retinal cells than the level of expression from a comparable AAV vector comprising a capsid polypeptide consisting of the amino acid sequence set forth in SEQ ID NO:1. The nucleic acid molecule can be DNA.

In another aspect, this document features a host cell comprising a nucleic acid molecule of either of the two preceding paragraphs. The host cell can express the vector. The host cell can express the polypeptide.

In another aspect, this document features a host cell comprising an AAV vector comprising an AAV capsid polypeptide, wherein the capsid polypeptide comprises the amino acid sequence of any one of SEQ ID NOs: 2-5. The capsid polypeptide can comprise the amino acid sequence of SEQ ID NO: 1 (or an alternative sequence that is the amino acid sequence set forth in SEQ ID NO:10 or that is an amino acid sequence at least 95%, at least 96%, at least 97%, at least 98%, or at least 99% identical to the amino acid sequence set forth in SEQ ID NO: 1) except that the amino acid sequence of any one of SEQ ID NOs: 2-5 is located between amino acid positions 587 and 588 of SEQ ID NO:1 (or the appropriate amino acid positions of the alternative sequence). The capsid polypeptide can comprise the amino acid sequence of SEQ ID NO:1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO: 1) except that the amino acid sequence of SEQ ID NO:5 is located between amino acid positions 587 and 588 of SEQ ID NO:1 (or the appropriate amino acid positions of the alternative sequence). The capsid polypeptide can comprise the amino acid sequence of SEQ ID NO: 1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO: 1 (or the appropriate amino acid positions of the alternative sequence) are replaced with the amino acid sequence of any one of SEQ ID NOs: 2-5. The capsid polypeptide can comprise the amino acid sequence of SEQ ID NO: 1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO: 1 (or the appropriate amino acid positions of the alternative sequence) are replaced with the amino acid sequence of SEQ ID NO: 2. The capsid polypeptide can comprise or consist of the amino acid sequence of any of SEQ ID NOs: 11-42. The vector can be an AAV2 vector. The vector can infect greater than 2.5 percent of retinal cells when a titer of at least 1×10⁷ of the vector is administered intravitreally to an eye of a human (or a non-human primate). The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in retinal cells than the level of expression from a comparable AAV vector comprising a capsid polypeptide consisting of the amino acid sequence set forth in SEQ ID NO:1. The host cell can be a retinal cell.

In another aspect, this document features a host cell comprising an AAV capsid polypeptide comprising the amino acid sequence of any one of SEQ ID NOs: 2-5. The polypeptide can comprise the amino acid sequence of SEQ ID NO: 1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO: 1) except that the amino acid sequence of any one of SEQ ID NOs: 2-5 is located between amino acid positions 587 and 588 of SEQ ID NO: 1 (or the appropriate amino acid positions of the alternative sequence). The polypeptide can comprise the amino acid sequence of SEQ ID NO: 1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO:1) except that the amino acid sequence of SEQ ID NO:5 is located between amino acid positions 587 and 588 of SEQ ID NO:1 (or the appropriate amino acid positions of the alternative sequence). The polypeptide can comprise the amino acid sequence of SEQ ID NO: 1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO: 1 (or the appropriate amino acid positions of the alternative sequence) are replaced with the amino acid sequence of any one of SEQ ID NOs: 2-5. The polypeptide can comprise the amino acid sequence of SEQ ID NO: 1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO: 1 (or the appropriate amino acid positions of the alternative sequence) are replaced with the amino acid sequence of SEQ ID NO:2. The capsid polypeptide can comprise or consist of the amino acid sequence of any of SEQ ID NOs: 11-42. An AAV vector comprising the polypeptide can infect greater than 2.5 percent of retinal cells when a titer of at least 1×10⁷ of the vector is administered intravitreally to an eye of a human (or a non-human primate). An AAV vector comprising the polypeptide can express more nucleic acid in retinal cells than the level of expression from a comparable AAV vector comprising a capsid polypeptide consisting of the amino acid sequence set forth in SEQ ID NO:1. The host cell can be a retinal cell.

In another aspect, this document features a composition comprising an AAV vector comprising an AAV capsid polypeptide, wherein the capsid polypeptide comprises the amino acid sequence of any one of SEQ ID NOs: 2-5. The capsid polypeptide can comprise the amino acid sequence of SEQ ID NO: 1 (or an alternative sequence that is the amino acid sequence set forth in SEQ ID NO:10 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs: 2-5 is located between amino acid positions 587 and 588 of SEQ ID NO:1 (or the appropriate amino acid positions of the alternative sequence). The capsid polypeptide can comprise the amino acid sequence of SEQ ID NO: 1 (or an alternative sequence that is the amino acid sequence set forth in SEQ ID NO:10 or that is an amino acid sequence at least 95%, at least 96%, at least 97%, at least 98%, or at least 99% identical to the amino acid sequence set forth in SEQ ID NO: 1) except that the amino acid sequence of SEQ ID NO:5 is located between amino acid positions 587 and 588 of SEQ ID NO: 1 (or the appropriate amino acid positions of the alternative sequence). The capsid polypeptide can comprise the amino acid sequence of SEQ ID NO: 1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO: 1 (or the appropriate amino acid positions of the alternative sequence) are replaced with the amino acid sequence of any one of SEQ ID NOs: 2-5. The capsid polypeptide can comprise the amino acid sequence of SEQ ID NO: 1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO: 1 (or the appropriate amino acid positions of the alternative sequence) are replaced with the amino acid sequence of SEQ ID NO: 2. The capsid polypeptide can comprise or consist of the amino acid sequence of any of SEQ ID NOs: 11-42. The vector can be an AAV2 vector. The vector can infect greater than 2.5 percent of retinal cells when a titer of at least 1×10⁷ of the vector is administered intravitreally to an eye of a human (or a non-human primate). The vector can comprise an exogenous nucleic acid encoding an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more nucleic acid in retinal cells than the level of expression from a comparable AAV vector comprising a capsid polypeptide consisting of the amino acid sequence set forth in SEQ ID NO:1, The composition can comprise from about 1×10⁷ to about 1×10¹⁴ of the vector. The composition can comprise phosphate buffered saline, Hank's Balanced Salt Solution, or Pluronic F68.

In another aspect, this document features a method for delivering an exogenous nucleic acid sequence to a retinal cell within a mammal. The method comprises (or consists essentially of, or consists of) contacting the retinal cell with an AAV vector comprising an AAV capsid polypeptide and the exogenous nucleic acid sequence, wherein the capsid polypeptide comprises the amino acid sequence of any one of SEQ ID NOs: 2-5, wherein the AAV vector infects the retinal cell, thereby delivering the exogenous nucleic acid sequence to the retinal cell. The capsid polypeptide can comprise the amino acid sequence of SEQ ID NO: 1 (or an alternative sequence that is the amino acid sequence set forth in SEQ ID NO:10 or that is an amino acid sequence at least 95%, at least 96%, at least 97%, at least 98%, or at least 99% identical to the amino acid sequence set forth in SEQ ID NO:1) except that the amino acid sequence of any one of SEQ ID NOs: 2-5 is located between amino acid positions 587 and 588 of SEQ ID NO: 1 (or the appropriate amino acid positions of the alternative sequence). The capsid polypeptide can comprise the amino acid sequence of SEQ ID NO:1 (or an alternative sequence that is the amino acid sequence set forth in SEQ ID NO:10 or that is an amino acid sequence at least 95%, at least 96%, at least 97%, at least 98%, or at least 99% identical to the amino acid sequence set forth in SEQ ID NO:1) except that the amino acid sequence of SEQ ID NO:5 is located between amino acid positions 587 and 588 of SEQ ID NO: 1 (or the appropriate amino acid positions of the alternative sequence). The capsid polypeptide can comprise the amino acid sequence of SEQ ID NO: 1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO: 1 (or the appropriate amino acid positions of the alternative sequence) are replaced with the amino acid sequence of any one of SEQ ID NOs: 2-5. The capsid polypeptide can comprise the amino acid sequence of SEQ ID NO: 1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO: 1 (or the appropriate amino acid positions of the alternative sequence) are replaced with the amino acid sequence of SEQ ID NO:2. The capsid polypeptide can comprise or consist of the amino acid sequence of any of SEQ ID NOs: 11-42. The mammal can be a human (or a non-human primate). The vector can be an AAV2 vector. The vector can infect greater than 2.5 percent of retinal cells when a titer of at least 1×10⁷ of the vector is administered intravitreally to an eye of a human (or a non-human primate). The exogenous nucleic acid sequence can encode an RNA or a polypeptide. The exogenous nucleic acid can encode an RNA. The RNA can be an siRNA or microRNA. The exogenous nucleic acid can encode a polypeptide. The polypeptide can be an ABCA4 polypeptide, a CRB1 polypeptide, an NPHP5 polypeptide, or an NR2E3 polypeptide. The vector can express more of the exogenous nucleic acid sequence in the retinal cell than the level of expression in a retinal cell from a comparable AAV vector comprising a capsid polypeptide consisting of the amino acid sequence set forth in SEQ ID NO:1. The method can comprise intravitreally administering a composition comprising the vector to the mammal, thereby contacting the retinal cell with the vector. The composition can comprise from about 1×10⁷ to about 1×10¹⁴ of the vector.

In another aspect, this document features a method for treating a retinal condition. The method comprises (or consists essentially of, or consists of) contacting retinal cells of a mammal having the retinal condition with AAV vectors comprising an AAV capsid polypeptide and an exogenous nucleic acid sequence, wherein the capsid polypeptide comprises the amino acid sequence of any one of SEQ ID NOs: 2-5, wherein the AAV vectors infect the retinal cells and drive expression of the exogenous nucleic acid sequence within the retinal cells, thereby treating the retinal condition. The mammal can be a human (or a non-human primate). The retinal condition can be selected from the group consisting of LCA, OCA1, retinitis pigmentosa, rod/cone dystrophy, cone dystrophy, Stargardt Disease, Usher syndrome, XLRP, and XLRS. The capsid polypeptide can comprise the amino acid sequence of SEQ ID NO: 1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO: 1) except that the amino acid sequence of any one of SEQ ID NOs: 2-5 is located between amino acid positions 587 and 588 of SEQ ID NO:1 (or the appropriate amino acid positions of the alternative sequence). The capsid polypeptide can comprise the amino acid sequence of SEQ ID NO: 1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO: 1) except that the amino acid sequence of SEQ ID NO:5 is located between amino acid positions 587 and 588 of SEQ ID NO: 1 (or the appropriate amino acid positions of the alternative sequence). The capsid polypeptide can comprise the amino acid sequence of SEQ ID NO:1 (or an alternative sequence that is the amino acid sequence set forth in SEQ ID NO:10 or that is an amino acid sequence at least 95%, at least 96%, at least 97%, at least 98%, or at least 99% identical to the amino acid sequence set forth in SEQ ID NO:1) except that the amino acids from position 585 to 590 of SEQ ID NO: 1 (or the appropriate amino acid positions of the alternative sequence) are replaced with the amino acid sequence of any one of SEQ ID NOs: 2-5. The capsid polypeptide can comprise the amino acid sequence of SEQ ID NO: 1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO: 1) except that the amino acids from position 585 to 590 of SEQ ID NO: 1 (or the appropriate amino acid positions of the alternative sequence) are replaced with the amino acid sequence SEQ ID NO:2. The capsid polypeptide can comprise or consist of the amino acid sequence of any of SEQ ID NOs: 11-42. The vectors can be AAV2 vectors. The vectors can infect greater than 2.5 percent of retinal cells when a titer of at least 1×10⁷ of the vectors is administered intravitreally to an eye of the mammal. The exogenous nucleic acid sequence can encode an RNA. The RNA can be an siRNA or a microRNA. The exogenous nucleic acid can encode a polypeptide. The polypeptide can be an ABCA4 polypeptide, a CRB1 polypeptide, an NPHP5 polypeptide, and an NR2E3 polypeptide. The vectors can express more of the exogenous nucleic acid sequence in the retinal cells than the level of expression in retinal cells from a comparable AAV vector comprising a capsid polypeptide consisting of the amino acid sequence set forth in SEQ ID NO:1. The method can comprise intravitreally administering a composition comprising the vectors to the mammal, thereby contacting the retinal cells with the vectors. The composition can comprise from about 1×10⁷ to about 1×10¹⁴ of the vectors.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and methods are described below. All publications, patent applications, patents, and other references mentioned herein are incorporated by reference in their entirety. In case of conflict, the present specification, including definitions will control. In addition, the materials, methods, and examples are illustrative only and not intended to be limiting.

The details of one or more embodiments of the invention are set forth in the accompanying description below. Other features, objects, and advantages of the invention will be apparent from the description and from the claims.

### DESCRIPTION OF DRAWINGS

FIG. 1 is a listing of SEQ ID NOs: 11-76.

FIG. 2 is a diagram of AAV vectors that include a wild type AAV2 Rep polypeptide and an indicated AAV2 capsid polypeptide engineered to include an insert sequence (e.g., any one of SEQ ID NOs: 2-5 or a sequence of Formula A) located between positions 587 and 588 (using SEQ ID NO: 1 numbering), according to some embodiments.

FIG. 3 is a diagram of AAV vectors that include a mutant AAV2 Rep polypeptide (AAV2-MIT-REP) and an indicated AAV2 capsid polypeptide engineered to include an insert sequence (e.g., any one of SEQ ID NOs: 2-5 or a sequence of Formula A) located between positions 587 and 588 (using SEQ ID NO: 1 numbering), according to some embodiments.

FIG. 4 is a diagram of AAV vectors that include a wild type AAV2 Rep polypeptide and an indicated AAV2 capsid polypeptide engineered to include an insert sequence (e.g., any one of SEQ ID NOs: 2-5 or a sequence of Formula A) as a replacement of amino acid residues at positions 585 to 590 (using SEQ ID NO: 1 numbering), according to some embodiments.

FIG. 5 is a diagram of AAV vectors that include a mutant AAV2 Rep polypeptide (AAV2-MIT-REP) and an indicated AAV2 capsid polypeptide engineered to include an insert sequence (e.g., any one of SEQ ID NOs: 2-5 or a sequence of Formula A) as a replacement of amino acid residues at positions 585 to 590 (using SEQ ID NO:1 numbering), according to some embodiments.

### DETAILED DESCRIPTION

This document provides AAV vectors (e.g., AAV2 vectors). For example, this document provides AAV vectors (e.g., AAV2 vectors) containing a capsid polypeptide that includes an amino acid sequence set forth in Table 1 (or a variant thereof) or Formula A. Any appropriate AAV vector can be designed to include a capsid polypeptide described herein (e.g., a capsid polypeptide that includes an amino acid sequence set forth in Table 1 (or a variant thereof) or Formula A). For example, AAV2, AAV8, and AAV9 can be designed to include a capsid polypeptide that includes an amino acid sequence set forth in Table 1 (or a variant thereof) or Formula A. In some cases, an AAV2 having an ACG start codon for the AAV Rep polypeptides (e.g., AAV2 Rep78 and Rep68 polypeptides; see, e.g., SEQ ID NOs: 75-76) instead of an ATG start codon (e.g., an AAV2-MIT-REP) can be designed to include a capsid polypeptide that includes an amino acid sequence set forth in Table 1 (or a variant thereof) or Formula A.

Any appropriate AAV capsid polypeptide can be designed to include an amino acid sequence set forth in Table 1 (or a variant thereof) or Formula A. For example, AAV2, AAV6, AAV8 and AAV9 capsid polypeptides can be designed to include an amino acid sequence set forth in Table 1 (or a variant thereof) or Formula A. In some cases, an AAV2 capsid polypeptide can be designed to include an amino acid sequence set forth in Table 1 (or a variant thereof) or Formula A. In some cases, an AAV2 capsid polypeptide having the following amino acid sequence can be designed to include an amino acid sequence set forth in Table 1 (or a variant thereof) or Formula A:

(SEQ ID NO: 1)
MAADGYLPDWLEDTLSEGIRQWWKLKPGPPPPKPAERHKDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
VEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNT
PSGTTTQSRLQFSQAGASDIRDQSRNWLPGPCYRQQRVSKTSADNNNSEY
SWTGATKYHLNGRDSLVNPGPAMASHKDDEEKFFPQSGVLIFGKQGSEKT
NVDIEKVMITDEEEIRTTNPVATEQYGSVSTNLQRGNRQAATADVNTQGV
LPGMVWQDRDVYLQGPIWAKIPHTDGHFHPSPLMGGFGLKHPPPQILIKN
TPVPANPSTTFSAAKFASFITQYSTGQVSVEIEWELQKENSKRWNPEIQY
TSNYNKSVNVDFTVDTNGVYSEPRPIGTRYLTRNL.

The two bold amino acid residues are at positions 587 and 588, and the underlined amino acids are at positions 585 to 590.

In some cases, an AAV capsid polypeptide (e.g., an AAV2 capsid polypeptide) having the following amino acid sequence can be designed to include an amino acid sequence set forth in Table 1 (or a variant thereof) or Formula A:

(SEQ ID NO: 10)
MAADGYLPDWLEDTLSEGIRQWWKLKPGPPPPKPAERHKDDSRGLVLPGY
KYLGPFNGLDKGEPVN [symbol] ADAAALEHDKAYDRQLDSGDNPYLKYNHADAE
FQERLKEDTSFGGNLGRAVFQAKKRVLEPLGLVEEPVKTAPGKKRPVEHS
PVEPDSSSGTGKAGQQPARKRLNFGQTGDADSVPDPQPLGQPPAAPSGLG
TNTMATGSGAPMADNNEGADGVGNSSGNWHCDSTWMGDRVITTSTRTWAL
PTYNNHLYKQISSQSGASNDNHYFGYSTPWGYFDFNRFHCHFSPRDWQRL
INNNWGFRPKRLNFKLFNIQVKEVTQNDGTTTIANNLTSTVQVFTDSEYQ
LPYVLGSAHQGCLPPFPADVFMVPQYGYLTLNNGSQAVGRSSFYCLEYFP
SQMLRTGNNFTFSYTFEDVPFHSSYAHSQSLDRLMNPLIDQYLYYLSRTN
TPSGTTTQSRLQFSQAGASDIRDQSRNWLPGPCYRQQRVSKTSADNNNSE
YSWTGATKYHLNGRDSLVNPGPAMASHKDDEEKFFPQSGVLIFGKQGSEK
TNVDIEKVMITDEEEIRTTNPVATEQYGSVSTNLQRGNRQAATADVNTQG
VLPGMVWQDRDVYLQGPIWAKIPHTDGHFHPSPLMGGFGLKHPPPQILIK
NTPVPANPSTTFSAAKFASFITQYSTGQVSVEIEWELQKENSKRWNPEIQ
YTSNYNKS [symbol] NVDFTVDINGVYSEPRPIGTRYLTRNL, wherein X₁
is E or A, and wherein X₂ is V or I.

In some cases, an AAV capsid polypeptide (e.g., an AAV2 capsid polypeptide) that is at least 95%, at least 96%, at least 97%, at least 98%, or at least 99% identical to the amino acid sequence set forth in SEQ ID NO: 1 can be designed to include an amino acid sequence set forth in Table 1 (or a variant thereof) or Formula A.

In some cases, certain AAV2 sequences contemplated herein can include modifications or mutations of SEQ ID NO: 1 such as a V708I and/or E67A substitution.

When designing an AAV capsid polypeptide (e.g., an AAV2 capsid polypeptide) to include an amino acid sequence set forth in Table 1 (or a variant thereof) or Formula A, that included amino acid sequence can be located at any appropriate location along the AAV capsid polypeptide (e.g., the AAV2 capsid polypeptide). For example, an amino acid sequence set forth in Table 1 (or a variant thereof) or Formula A such as any one of SEQ ID NOs: 2-5 can be located between the naturally-occurring amino acid residues at positions 587 and 588 of an AAV capsid polypeptide (e.g., an AAV2 capsid polypeptide), can be located between the naturally-occurring amino acid residues at positions 452 and 453 of an AAV capsid polypeptide (e.g., an AAV2 capsid polypeptide), or can be located between the naturally-occurring amino acid residues at positions 453 and 454 of an AAV capsid polypeptide (e.g., an AAV2 capsid polypeptide).

TABLE 1
Amino acid sequences that can be inserted into an
AAV capsid polypeptide.

| | SEQ | | SEQ |
|---|---|---|---|
| Amino Acid | ID | Nucleic Acid Sequence encoding | ID |
| Sequence | NO: | the Amino Acid Sequence | NO: |
| EGSGRN | 2 | GAGGGCAGCGGCCGGAAC | 6 |
| AEGSGRN | 3 | GCAGAGGGCAGCGGCCGGAAC | 7 |
| LAEGSGRN | 4 | CTAGCAGAGGGCAGCGGCCGGAAC | 8 |
| LAEGSGRNA | 5 | CTAGCAGAGGGCAGCGGCCGGAACGCT | 9 |

As described herein, an AAV vector can be designed to have an AAV capsid polypeptide that includes an amino acid sequence insert of Formula A. For example, an AAV vector can be designed to have an AAV capsid polypeptide of SEQ ID NO: 1 (or an alternative sequence that is the amino acid sequence set forth in SEQ ID NO:10 or that is an amino acid sequence at least 95%, at least 96%, at least 97%, at least 98%, or at least 99% identical to the amino acid sequence set forth in SEQ ID NO:1) that includes an amino acid sequence insert of Formula A located between amino acid positions 587 and 588 of SEQ ID NO: 1 (or the appropriate amino acid positions of the alternative sequence). Formula A can be as follows:

(SEQ ID NO: 2)
-L1-EGSGRN -L2-,

wherein L1 and L2 are each independently optional amino acid linkers having one, two, or three amino acids. In some cases, L1, L2, or both L1 and L2 can be absent. In some cases, L1 can be one amino acid X1, two amino acids X2-X1, or three amino acids X3-X2-X1. When X1 is present, it can be an amino acid residue selected from the group consisting of A, V, I, and L. When X2 is present, it can be an amino acid residue selected from the group consisting of A, V, I, and L. When X3 is present, it can be an amino acid residue selected from the group consisting of A, V, I, and L. In some cases, L2 can be one amino acid Z1, two amino acids Z1-Z2, or three amino acids Z1-Z2-Z3. When Z1 is present, it can be an amino acid residue selected from the group consisting of A, V, I, and L. When Z2 is present, it can be an amino acid residue selected from the group consisting of A, V, I, and L. When Z3 is present, it can be an amino acid residue selected from the group consisting of A, V, I, and L. Examples of an L1 linkers include, without limitation, A, V, I, L, AA, AV, AI, AL, VA, VV, VI, VL, IA, IV, II, IL, LA, LV, L1, LL, AAA, AAV, AAI, AAL, AVA, AVV, AVI, AVL, AIA, AIV, AII, AIL, ALA, ALV, ALI, ALL, VAA, VAV, VAI, VAL, VVA, VVV, VVI, VVL, VIA, VIV, VII, VIL, VLA, VLV, VLI, VLL, IAA, IAV, IAI, IAL, IVA, IVV, IVI, IVL, IIA, IIV, III, IIL, ILA, ILV, ILI, ILL, LAA, LAV, LAI, LAL, LVA, LVV, LVI, LVL, LIA, LIV, LII, LIL, LLA, LLV, LLI, and LLL. Examples of an L2 linkers include, without limitation, A, V, I, L, AA, AV, AI, AL, VA, VV, VI, VL, IA, IV, II, IL, LA, LV, L1, LL, AAA, AAV, AAI, AAL, AVA, AVV, AVI, AVL, AIA, AIV, AII, AIL, ALA, ALV, ALI, ALL, VAA, VAV, VAI, VAL, VVA, VVV, VVI, VVL, VIA, VIV, VII, VIL, VLA, VLV, VLI, VLL, IAA, IAV, IAI, IAL, IVA, IVV, IVI, IVL, IIA, IIV, III, IIL, ILA, ILV, ILI, ILL, LAA, LAV, LAI, LAL, LVA, LVV, LVI, LVL, LIA, LIV, LII, LIL, LLA, LLV, LLI, and LLL.

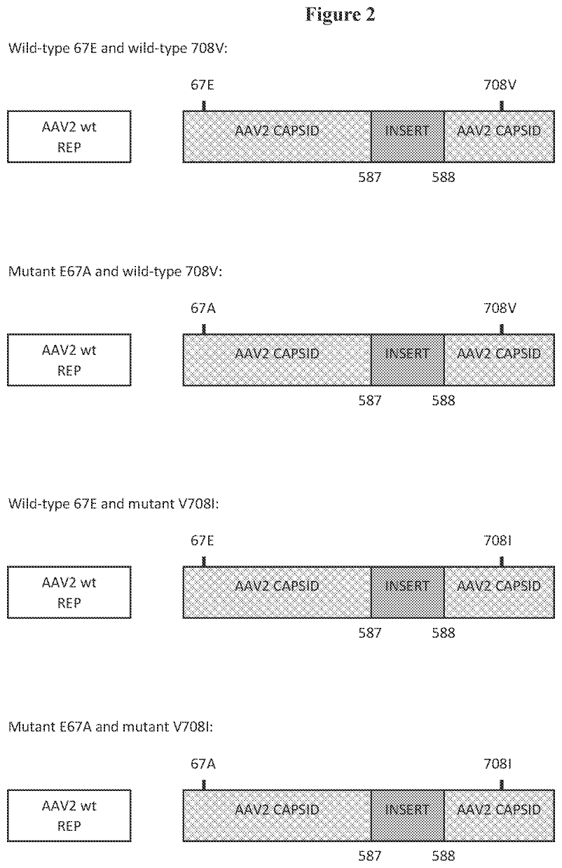

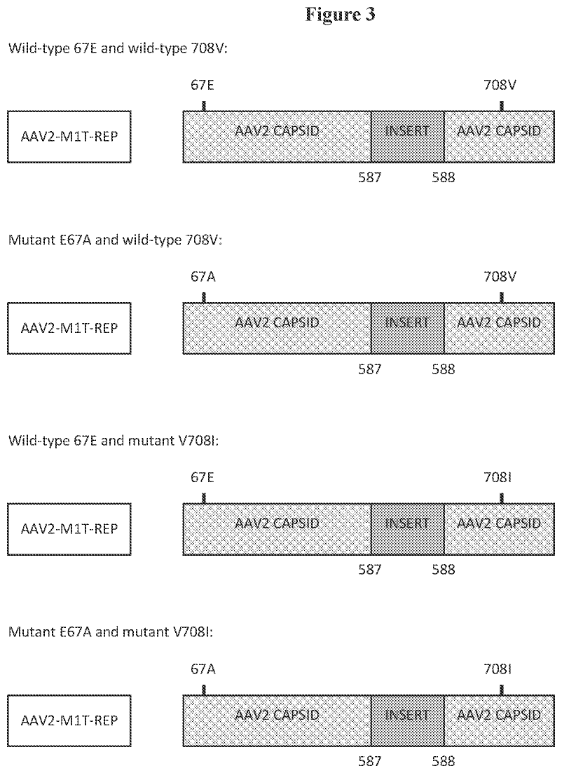

In some cases, an AAV2 capsid polypeptide provided herein can have the sequence set forth in SEQ ID NO: 1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO:1) with an amino acid sequence set forth in Table 1 (or a variant thereof) or Formula A inserted between asparagine-587 and arginine-588 (or the appropriate amino acid positions of the alternative sequence) (see, e.g., FIGS. 2-3). In some cases, an AAV2 capsid polypeptide provided herein can have the sequence set forth in SEQ ID NO:1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO: 1) with an amino acid sequence set forth in any one of SEQ ID NOs: 2-5 (or a variant thereof) inserted between asparagine-587 and arginine-588 (or the appropriate amino acid positions of the alternative sequence).

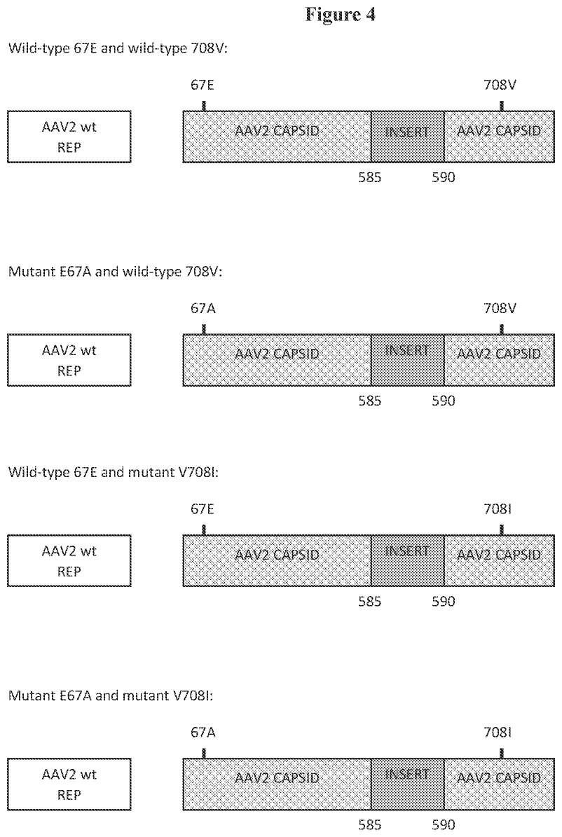

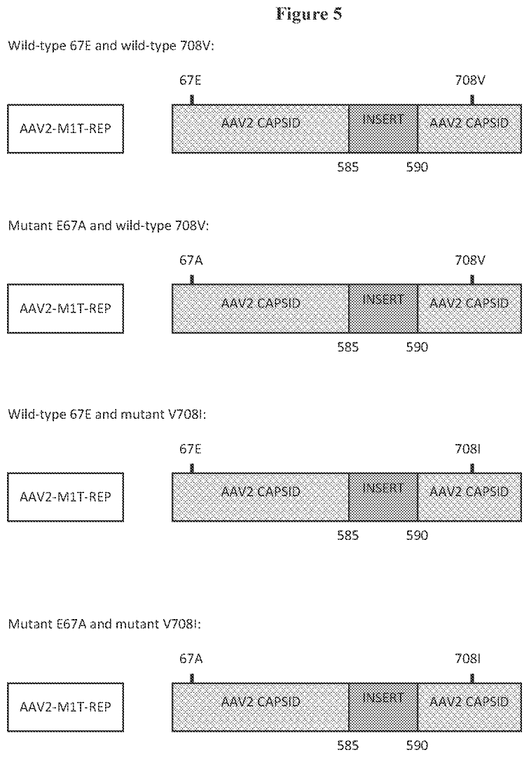

In some cases, when designing an AAV capsid polypeptide (e.g., an AAV2 capsid polypeptide) to include an amino acid sequence set forth in Table I (or a variant thereof) or Formula A, that included amino acid sequence can be used to replace one or more naturally-occurring amino acid residues located at any appropriate location along the AAV capsid polypeptide (e.g., the AAV2 capsid polypeptide). For example, an amino acid sequence set forth in Table 1 (or a variant thereof) or Formula A such as any one of SEQ ID NOs: 2-5 can be used to replace the naturally-occurring amino acid residues at positions 585 to 590 of an AAV capsid polypeptide (e.g., an AAV2 capsid polypeptide) (see, e.g., FIGS. 4-5).

In some cases, an AAV2 capsid polypeptide provided herein can have the sequence set forth in SEQ ID NO: 1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO: 1) except that the amino acid residues at positions 585 to 590 (or the appropriate amino acid positions of the alternative sequence) are replaced with an amino acid sequence set forth in Table 1 (or a variant thereof) or Formula A. In some cases, an AAV2 capsid polypeptide provided herein can have the sequence set forth in SEQ ID NO:1 (or an alternative sequence that is the amino acid sequence set forth in SEQ ID NO: 10 or that is an amino acid sequence at least 95%, at least 96%, at least 97%, at least 98%, or at least 99% identical to the amino acid sequence set forth in SEQ ID NO: 1) with the exception that amino acid residues 585 to 590 (or the appropriate amino acid positions of the alternative sequence) are replaced with the amino acid sequence set forth in any one of SEQ ID NOs: 2-5 (or a variant thereof).

In some cases, an AAV capsid polypeptide (e.g., an AAV2 capsid polypeptide) can be designed to include two or more amino acid sequences set forth in Table 1 (or a variant thereof) or Formula A. For example, an AAV capsid polypeptide (e.g., an AAV2 capsid polypeptide) can be designed to include two or three amino acid sequences set forth in Table 1 (or a variant thereof) or Formula A.

As described herein, an AAV capsid polypeptide (e.g., an AAV2 capsid polypeptide) can be designed to include an amino acid sequence set forth in Table 1 (or a variant thereof) or Formula A. A variant of an amino acid sequence set forth in Table I refers to an amino acid sequence that is identical to that amino acid sequence set forth in Table 1 except that it has one, two, or three amino acid additions, deletions, substitutions, or combinations thereof. For example, a variant of SEQ ID NO:2 can be SEQ ID NO:2 except that it has one, two, or three amino acid additions, deletions, substitutions, or combinations thereof. In some cases, a variant provided herein can be the amino acid sequence set forth in any one of SEQ ID NOs: 2-5 except that it contains one, two, or three amino acid additions. In some cases, a variant provided herein can be the amino acid sequence set forth in any one of SEQ ID NOs: 2-5 except that it contains one, two, or three amino acid deletions. In some cases, a variant provided herein can be the amino acid sequence set forth in any one of SEQ ID NOs: 2-5 except that it contains one, two, or three amino acid substitutions. In some cases, a variant provided herein can be the amino acid sequence set forth in any one of SEQ ID NOs: 2-5 except that it contains one amino acid addition, deletion, or substitution. In some cases, a variant provided herein can be the amino acid sequence set forth in any one of SEQ ID NOs: 2-5 except that it contains two amino acid additions, deletions, substitutions, or a combination thereof. In some cases, a variant provided herein can be the amino acid sequence set forth in any one of SEQ ID NOs: 2-5 except that it contains three amino acid additions, deletions, substitutions, or a combination thereof.

In some cases, an amino acid substitution present in a variant can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some cases, an amino acid substitution present in a variant can be a non-conservative amino acid substitution. Non-conservative amino acid substitutions can be made by substituting one amino acid residue for another amino acid residue having a dissimilar side chain. Examples of non-conservative substitutions include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

The percent sequence identity between a particular amino acid sequence and an amino acid sequence referenced by a particular sequence identification number is determined as follows. First, an amino acid sequence is compared to the sequence set forth in a particular sequence identification number using the BLAST 2 Sequences (Bl2seq) program from the stand-alone version of BLASTZ containing BLASTP version 2.0.14. This stand-alone version of BLASTZ can be obtained from Fish & Richardson's web site (e.g., www.fr.com/blast/) or the U.S. government's National Center for Biotechnology Information web site (www.ncbi.nlm.nih.gov). Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq-i c:\seq1.txt-j c:\seq2.txt-p blastp-o c:\output.txt. If the two compared sequences share homology, then the designated output file will present those regions of homology as aligned sequences. If the two compared sequences do not share homology, then the designated output file will not present aligned sequences. Once aligned, the number of matches is determined by counting the number of positions where an identical amino acid residue is presented in both sequences. A matched position refers to a position in which an identical amino acid residue occurs at the same position in aligned sequences. The percent sequence identity is determined by dividing the number of matches by the length of the sequence set forth in the identified sequence (e.g., SEQ ID NO:1), followed by multiplying the resulting value by 100. For example, an amino acid sequence that bas 725 matches when aligned with the sequence set forth in SEQ ID NO:1 is 98.6 percent identical to the sequence set forth in SEQ ID NO: 1 (i.e., 725÷735×100=98.6). It is noted that the percent sequence identity value is rounded to the nearest tenth. For example, 78.11, 78.12, 78.13, and 78.14 is rounded down to 78.1, while 78.15, 78.16, 78.17, 78.18, and 78.19 is rounded up to 78.2. It also is noted that the length value will always be an integer.

Methods for generating an amino acid sequence variant can include site-specific mutagenesis or random mutagenesis (e.g., by PCR) of a nucleic acid encoding an AAV capsid polypeptide. See, for example, Zoller, Curr. Opin. Biotechnol. 3:348-354 (1992).

The AAV vectors (e.g., AAV2 vectors) described herein can be designed to include one or more exogenous nucleic acid sequences. For example, an AAV vector (e.g., an AAV2 vector) described herein can be designed to include an exogenous nucleic acid sequence that encodes an RNA of interest and/or a polypeptide of interest. An exogenous nucleic acid sequence can be designed to encode any appropriate RNA of interest. Examples of RNAs of interest that can be encoded by an exogenous nucleic acid sequence designed to be included within an AAV vector provided herein include, without limitation, siRNAs, RNA components for gene editing, and microRNAs. In some cases, an RNA of interest that can be encoded by an exogenous nucleic acid sequence included within an AAV vector provided herein can be SIRNA-027 to treat, e.g., sub-foveal CNVM secondary to age-related macular degeneration (see, e.g., NCT00363714), Cand5/Bevasiranib to treat, e.g., diabetic macular edema (see, e.g., NCT00306904), PF-04523655 to treat, e.g., diabetic macular edema (see, e.g., NCT01445899), QPI-1007 to treat, e.g., optic nerve atrophy in NAION (see, e.g., NCT01064505), Aganirsen to treat, e.g., ischemic CRVO to prevent neovascular glaucoma (see, e.g., NCT02947867), QR-421a to treat, e.g., retinitis pigmentosa/Usher syndrome type 2 (see, e.g., NCT03780257), QR-1123 to treat, e.g., autosomal dominant retinitis pigmentosa (see, e.g., NCT04123626), IONIS-FB-LRx to treat, e.g., geographic atrophy secondary to age-related macular degeneration (see, e.g., NCT03815825), or Sepofarsen/QR-110 to treat, e.g., Leber's congenital amaurosis (see, e.g., NCT03913143).

An exogenous nucleic acid sequence can be designed to encode any appropriate polypeptide of interest. Examples of polypeptides of interest that can be encoded by an exogenous nucleic acid sequence designed to be included within an AAV vector provided herein include, without limitation, therapeutic polypeptides, trophic factor polypeptides, gene editing polypeptides (e.g., a Cas9 polypeptide, a TALEN polypeptide, or a zinc finger polypeptide), enzymes, optogenetic tool polypeptides (e.g., a ChR polypeptide, an NhpR polypeptide, or a ReachR polypeptide), antibodies, antibody domains (e.g., VH domains), cytokines, anti-angiogenic polypeptides, and neuroprotective polypeptides. Examples of polypeptides of interest that can be encoded by an exogenous nucleic acid sequence designed to be included within an AAV vector provided herein include, without limitation, an ABCA4 polypeptide, a CRB1 polypeptide, an NPHP5 polypeptide, an NR2E3 polypeptide, a PDE6A polypeptide, a PDE6B polypeptide, a PDE6C polypeptide, a PRPF31 polypeptide, a RPE65 polypeptide, a RPGR polypeptide, a RSI polypeptide, a TYR polypeptide, a USH2A polypeptide, a MYO7A polypeptide, an REP1 polypeptide, an OPN1LW polypeptide, an OPN1MW polypeptide, a CNGA3 polypeptide, a CNGB3 polypeptide, a GUCY2D polypeptide, a GACA1A polypeptide, a GNAT2 polypeptide, a PDE6H polypeptide, a PROM1 polypeptide, a PRPH2 polypeptide, a CRX polypeptide, an NPHP5 polypeptide, an EYS polypeptide, an ND4 polypeptide, a CLN1-14 polypeptide (e.g., a CLN3 polypeptide, a CLN5 polypeptide, a CLN6 polypeptide, or a CLN8 polypeptide), an NYX polypeptide, a GRM6 polypeptide, a TRPM1 polypeptide, a GPR179 polypeptide, an LRIT3 polypeptide, a glial cell derived neurotrophic factor (GDNF) polypeptide, a brain-derived neurotrophic factor (BDNF) polypeptide, a fibroblast growth factor (FGF) polypeptide, a truncated rod-derived cone viability factor (RdCVF) polypeptide, a full-length rod-derived cone viability factor (RdCVFL) polypeptide, an X-linked inhibitor of apoptosis (XIAP) polypeptide, a soluble fms-related receptor tyrosine kinase 1 (sFLT) polypeptide, a CYP4V2 polypeptide, a palmitoyl protein thioesterase I polypeptide, a tripeptidyl peptidase 1 polypeptide, a DNAJC5 polypeptide, a MFSD8 polypeptide, a cathepsin D polypeptide, a granulin polypeptide, an ATP13A2 polypeptide, a cathepsin F polypeptide, a KCTD7 polypeptide, a “P” gene polypeptide, a TRP1 polypeptide, a MATP (SLC45A2) polypeptide, a SLC24A5 polypeptide, a LRMDA polypeptide, a GPR143 polypeptide, an RPGR-exon 1-ORF15 polypeptide, an USH2b polypeptide, an USH1C polypeptide, a CDH23 polypeptide, a PCDH15 polypeptide, a SANS polypeptide, an USH1H polypeptide, a CIB2 polypeptide, an USH1K polypeptide, an ADGRV1 polypeptide, a WHRN polypeptide, a PDZD7 polypeptide, a CLRN1 polypeptide, a HARS polypeptide, an RP2 polypeptide, a FAM161 polypeptide, a DLK polypeptide, a RHO polypeptide, a CHM polypeptide, a BEST1 polypeptide, a RPI polypeptide, an OPA1 polypeptide, a CEP290 polypeptide, a RDH12 polypeptide, a CACNA IF polypeptide, a BBS1 polypeptide, a FAM161A polypeptide, a CERKL polypeptide, a PRPF8 polypeptide, a RP1L1 polypeptide, a SNRNP200 polypeptide, an IMPG2 polypeptide, a CDHR1 polypeptide, an IMPDH1 polypeptide, a CNGB1 polypeptide, a MERTK polypeptide, a KCNV2 polypeptide, an AIPL1 polypeptide, a RPGRIP1 polypeptide, a TULP1 polypeptide, a C2ORF71 (aka PCARE) polypeptide, a MAK polypeptide, a TIMP3 polypeptide, a GUCA1A polypeptide, an ALMS1 polypeptide, a BBS10 polypeptide, an IFT140 polypeptide, a CNGA1 polypeptide, a NMNAT1 polypeptide, a COL2A1 polypeptide, an EFEMP1 polypeptide, a WFS1 polypeptide, a RDH5 polypeptide, a PRPF3 polypeptide, a LRP5 polypeptide, a TOPORS polypeptide, a DHDDS polypeptide, a LCA5 polypeptide, an IQCB1 polypeptide, a RP9 polypeptide, an ATXN7 polypeptide, a BBS2 polypeptide, a SAG RLBP1 polypeptide, a ND6 (MT-ND6) polypeptide, a C1QTNF5 polypeptide, a VPS13B polypeptide, a KIF11 polypeptide, a MT-TL1 polypeptide, a KLHL7 polypeptide, an ACO2 polypeptide, a C21orf2 (aka CFAP410) polypeptide, an AHI1 polypeptide, a KIZ polypeptide, a SPATA7 polypeptide, a TTLL5 polypeptide, an HGSNAT polypeptide, a NRL polypeptide, an OAT polypeptide, a FLVCR1 polypeptide, an ABCC6 polypeptide, a LRAT polypeptide, a CEP78 polypeptide, a CDH3 polypeptide, a FZD4 polypeptide, a BBS12 polypeptide, an HK1 polypeptide, a PRDM13 polypeptide, an ADAM9 polypeptide, a BBS7 polypeptide, a CABP4 polypeptide, an ABHD12 polypeptide, a COL18A1 polypeptide, a MFRP polypeptide, a RIMS1 polypeptide, a ROM1 polypeptide, a BBS4 polypeptide, an IMPG1 polypeptide, an INPP5E polypeptide, a VCAN polypeptide, a POC1B polypeptide, a RAX2 polypeptide, a TSPAN12 polypeptide, a CACNA2D4 polypeptide, a JAG1 polypeptide, a MKKS polypeptide, a NPHP4 polypeptide, a BBS9 polypeptide, a COL11A1 polypeptide, an ELOVL4 polypeptide, a NDP polypeptide, a NPHP1 polypeptide, a RGR polypeptide, a BBS5 polypeptide, a WDR19 polypeptide, a C8ORF37 polypeptide, a CTNNA1 polypeptide, a LAMP2 polypeptide, a PEX1 polypeptide, a PHYH polypeptide, an ATF6 polypeptide, a PRPS1 polypeptide, a SEMA4A polypeptide, an ARL6 polypeptide, a CNNM4 polypeptide, an OTX2 polypeptide, a PRPF6 polypeptide, a RBP3 polypeptide, a PNPLA6 polypeptide, a SLC24A1 polypeptide, an USH1G polypeptide, a PITPNM3 polypeptide, a TTC8 polypeptide, an ARSG polypeptide, a CWC27 polypeptide, a DRAM2 polypeptide, a PRCD polypeptide, a REEP6 polypeptide, a SSBP1 polypeptide, a LAMAI polypeptide, a RAB28 polypeptide, a ZNF408 polypeptide, a GNAT1 polypeptide, an IDH3A polypeptide, a PDE6G polypeptide, a PEX6 polypeptide, a TUB polypeptide, a CEP250 polypeptide, a FSCN2 polypeptide, a GRK1 polypeptide, a RBP4 polypeptide, a RD3 polypeptide, an AGBL5 polypeptide, a CAPN5 polypeptide, an IFT172 polypeptide, a KCNJ13 polypeptide, a PAX2 polypeptide, a CC2D2A polypeptide, a HMCN1 polypeptide, a MT-ATP6 polypeptide, a RCBTB1 polypeptide, an ARL2BP polypeptide, a CA4 polypeptide, a DFNB31 polypeptide, a GNB3 polypeptide, a MMACHC polypeptide, a PRPF4 polypeptide, a RGS9 polypeptide, an ARHGEF18 polypeptide, a KIAA1549 polypeptide, a MKS1 polypeptide, a MTTP (not MT-TP) polypeptide, a PLK4 polypeptide, a RPGRIP1L polypeptide, a SDCCAG8 polypeptide, a SRD5A3 polypeptide, a TUBB4B polypeptide, an ADAMTS18 polypeptide, an ARL3 polypeptide, a COL11A2 polypeptide, a MVK polypeptide, a NBAS polypeptide, an OFD1 polypeptide, a P3H2 polypeptide, a RGS9BP polypeptide, a CSPP1 polypeptide, an ITM2B polypeptide, a PANK2 polypeptide, a PEX7 polypeptide, a POMGNT1 polypeptide, a SLC4A7 polypeptide, a TMEM231 polypeptide, a TRNT1 polypeptide, a TUBGCP6 polypeptide, a ZNF513 polypeptide, an AFG3L2 polypeptide, an ARL13B polypeptide, a C5ORF42 (aka CPLANE1) polypeptide, a COL9A1 polypeptide, a CTSD polypeptide, a DTHD1 polypeptide, a DYNC2H1 polypeptide, an IFT81 polypeptide, a KIAA0586 polypeptide, a MFN2 polypeptide, a NPHP3 polypeptide, a PCYT1A polypeptide, a PEX12 polypeptide, a PLA2G5 polypeptide, a POC5 polypeptide, a SCAPER polypeptide, a SLC25A46 polypeptide, a TMEM237 polypeptide, a TRAF3IP1 polypeptide, a TTC21B polypeptide, a TUBGCP4 polypeptide, an ADIPOR1 polypeptide, a CEP164 polypeptide, a CLCC1 polypeptide, a COL9A2 polypeptide, a CTNNB1 polypeptide, a DHX38 polypeptide, a GNPTG polypeptide, a GRN polypeptide, a GUCA1B polypeptide, an IFT27 polypeptide, an IFT74 polypeptide, a KIAA0556 polypeptide, a LRP2 polypeptide, a MAPKAPK3 polypeptide, a MIR204 polypeptide, a MT-ND3 polypeptide, a MT-RNR1 polypeptide, a MT-TS2 polypeptide, a ND5 (MT-ND5) polypeptide, a NEK2 polypeptide, an OPN1SW polypeptide, a PEX13 polypeptide, a PEX2 polypeptide, a RHBDD2 polypeptide, a SAMD11 polypeptide, a SCLT1 polypeptide, a SLC7A14 polypeptide, a TCIN1 polypeptide, a TCTN2 polypeptide, a TLCD3B polypeptide, a TREX1 polypeptide, a TTPA polypeptide, an UNCI19 polypeptide, a WDPCP polypeptide, an ACBD5 polypeptide, an AHR polypeptide, an ARMC9 polypeptide, an ASRGL1 polypeptide, an ATOH7 polypeptide, a B9D1 polypeptide, a B9D2 polypeptide, a BBIP1 polypeptide, a C12ORF65 polypeptide, a C2CD3 polypeptide, a CSAR2 polypeptide, a CCDC188 polypeptide, a CCT2 polypeptide, a CEP104 polypeptide, a CEP120 polypeptide, a CEP19 polypeptide, a CEP41 polypeptide, a CISD2 polypeptide, a CLUAP1 polypeptide, a COL9A3 polypeptide, a CRB2 polypeptide, a CTC1 polypeptide, a DACT2 polypeptide, a DDR1 polypeptide, an ENSA polypeptide, an ESPN polypeptide, an EXOSC2 polypeptide, a FBN3 polypeptide, a GDF6 polypeptide, a GPR125 polypeptide, a HKDC1 polypeptide, a HMX1 polypeptide, an IDH3B polypeptide, an IFT43 polypeptide, an IFT80 polypeptide, an INVS polypeptide, a KIAA0753 polypeptide, a KIF3B polypeptide, a KIF7 polypeptide, a LRRTM4 polypeptide, a LZTFL1 polypeptide, a MT-ATP8 polypeptide, a MT-CO1 polypeptide, a MT-CO2 polypeptide, a MT-CO3 polypeptide, a MT-CYB polypeptide, a MT-ND2 polypeptide, a MT-ND4L polypeptide, a MT-RNR2 polypeptide, a MT-TA polypeptide, a MT-TC polypeptide, a MT-TD polypeptide, a MT-TE polypeptide, a MT-TF polypeptide, a MT-TG polypeptide, a MT-TH polypeptide, a MT-TI polypeptide, a MT-TK polypeptide, a MT-TL2 polypeptide, a MT-TM polypeptide, a MT-TN polypeptide, a MT-TP (Not MTTP) polypeptide, a MT-TQ polypeptide, a MT-TR polypeptide, a MT-TS1 polypeptide, a MT-TT polypeptide, a MT-TV polypeptide, a MT-TW polypeptide, a MT-TY polypeptide, a NEUROD1 polypeptide, a PDE6D polypeptide, a PEX10 polypeptide, a PEX11B polypeptide, a PEX14 polypeptide, a PEX16 polypeptide, a PEX19 polypeptide, a PEX26 polypeptide, a PEX3 polypeptide, a PEX5 polypeptide, a PGK1 polypeptide, a PISD polypeptide, a PPP2R3C polypeptide, a PROS1 polypeptide, a PSEN1 polypeptide, a RDH11 polypeptide, a RRM2B polypeptide, a SMARCA4 polypeptide, a SPP2 polypeptide, a TCTN3 polypeptide, a TEAD1 polypeptide, a TMEM107 polypeptide, a TMEM138 polypeptide, a TMEM216 polypeptide, a TMEM67 polypeptide, a TPP1 polypeptide, a TRIM32 polypeptide, a USP45 polypeptide, and a ZNF423 polypeptide.

In some cases, one or more AAV vectors provided herein can be designed to carry out gene editing within one or more cells (e.g., retinal cells). Such gene editing can result in a genomic modification of one or more cells. Examples of such genomic modifications include, without limitation, a targeted insertion of a nucleic acid encoding an RNA and/or polypeptide of interest into one or more cells, a targeted modification (e.g., targeted inactivation or knock-out) of a genomic sequence of one or more cells, and a targeted replacement of nucleic acid (e.g., nucleic acid encoding an RNA, a regulatory nucleic acid sequence, and/or nucleic acid encoding a polypeptide of interest) within one or more cells.

Any appropriate gene editing components can be engineered into one or more AAV vectors provided herein such that those one or more AAV vectors can be used to deliver the gene editing components to target cells (e.g., one or more retinal cells) within a mammal (e.g., a human or a non-human primate) in a manner effective to edit the genome of those cells. Typically, the gene editing components include, without limitation, a component that is capable of cleaving genomic nucleic acid at a desired location and an optional donor nucleic acid designed to be inserted into that desired location once it is cleaved. Any appropriate rare-cutting endonuclease can be used to cleave genomic nucleic acid at a desired location. Examples of such rare-cutting endonucleases include, without limitation, meganucleases, transcription activator-like effector (TALE) nucleases (TALENS™; Cellectis, Paris, France), zinc-finger-nucleases (ZFNs), and endonucleases of a clustered regularly interspaced short palindromic repeats (CRISPR)/Cas system (e.g., endonucleases of a CRISPR/Cas 9 system). See, e.g., Baker, Nature Methods, 9:23-26 (2012); International PCT Patent Application Publication No. WO 2004/067736; International PCT Patent Application Publication No. WO 2011/072246; U.S. Pat. No. 8,586,363; Porteus and Carroll, Nature Biotechnol., 23:967-973 (2005); Jinek et al., Science, 337:816-821 (2012); Mali et al., Science, 339:823-826 (2013); Li et al., Nature Biotechnology, 31 (8): 688-691 (2013); and Makarova et al., Nat. Rev. Microbiol., 9 (6): 467-477 (2011)).

In some cases, to facilitate gene replacement, two sequences in genomic nucleic acid of a cell (e.g., a retinal cell)—one on either side of a sequence to be removed—can be targeted for endonuclease cleavage. For example, a first target sequence adjacent to the 5′ end of a sequence to be removed and a second target sequence adjacent to the 3′ end of the sequence to be removed can be targeted by guide RNAs to enable Cas9 cleavage or can be targeted by TALENs designed to specifically recognize those targets. Delivery using one or more AAV vectors provided herein of (a) endonucleases targeted to the genomic DNA and (b) a donor nucleic acid construct can allow cleavage at both genomic targets, removal of the sequence between the genomic targets, and insertion of the donor sequence into the location of the deletion.

An AAV vector (e.g., an AAV2 vector) provided herein can include any appropriate promoter and/or other regulatory sequence (e.g., enhancers, transcription initiation sites, translation initiation sites, and termination signals) operably linked an exogenous nucleic acid sequence designed to be expressed. In some cases, a promoter used to drive expression can be a constitutive promotor, a regulatable promotor, a tissue-specific promoter, or a viral promotor. Examples of constitutive promotors that can be used as described herein include, without limitation, SV40 promotors, CMV promotors, and E1ALPHA promotors. Examples of regulatable promoters that can be used as described herein include, without limitation, inducible promotors and repressible promotors. Examples of tissue-specific promotors that can be used as described herein include, without limitation, rhodopsin promotors, cone arrestin promotors, and synapsin promotors. Examples of viral promotors that can be used as described herein include, without limitation, adenoviral promotors, vaccinia virus promotors, CMV promotors (e.g., immediate early CMV promotors), and AAV promoters.

In some cases, an AAV vector (e.g., an AAV2 vector) provided herein can include a total number of nucleotides up to about 5 kb. In some cases, an AAV vector (e.g., an AAV2 vector) provided herein can include a total number of nucleotides that is from about 1 kb to about 5 kb, from about 1 kb to about 4 kb, from about 1 kb to about 3 kb, from about 2 kb to about 5 kb, from about 2 kb to about 4 kb, from about 2 kb to about 3 kb, from about 3 kb to about 5 kb, from about 3 kb to about 4 kb, or from about 4 kb to about 5 kb.

An AAV vector (e.g., an AAV2 vector) described herein containing an AAV capsid polypeptide (e.g., an AAV2 capsid polypeptide) that includes an amino acid sequence set forth in Table 1 (or a variant thereof) or Formula A can have the ability to infect retinal cells (e.g., retinal ganglion cells) in vivo and deliver exogenous nucleic acid sequence to the infected retinal cells such that the infected retinal cells express the exogenous nucleic acid sequence. In some cases, an AAV vector (e.g., an AAV2 vector) provided herein can have the ability to infect and drive RNA expression of an exogenous nucleic acid sequence in more retinal cells of a mammal (e.g., a human or a non-human primate) when compared to wild-type AAV2. In some cases, an AAV vector (e.g., an AAV2 vector) provided herein can have the ability to drive a level of RNA expression of an exogenous nucleic acid sequence in retinal cells of a mammal (e.g., a human or a non-human primate) that is greater than the level of RNA expression of an exogenous nucleic acid sequence driven by a control AAV vector (e.g., wild-type AAV2) having an AAV capsid polypeptide that consists of the amino acid sequence set forth in SEQ ID NO: 1 in retinal cells of a control mammal (e.g., a control human or a control non-human primate).

Examples of retinal cells that can be infected by an AAV vector (e.g., an AAV2 vector) described herein containing an AAV capsid polypeptide (e.g., an AAV2 capsid polypeptide) that includes an amino acid sequence set forth in Table 1 (or a variant thereof) or Formula A include, without limitation, retinal ganglion cells, retinal pigment epithelium cells, photoreceptor cells, bipolar cells, amacrine cells, Muller glia, and horizontal cells.

This document also provides compositions containing one or more AAV vectors provided herein (e.g., one or more AAV2 vectors provided herein). For example, one or more AAV vectors provided herein (e.g., one or more AAV2 vectors provided herein) can be formulated as a pharmaceutical composition for administration to a mammal (e.g., a human or a non-human primate) to treat that mammal. In some cases, one or more AAV vectors provided herein (e.g., one or more AAV2 vectors provided herein) can be formulated as a pharmaceutical composition for administration to a mammal (e.g., a human or a non-human primate) to deliver an exogenous nucleic acid sequence to retinal cells (e.g., retinal ganglion cells) for expression within retinal cells. For example, an AAV vector (e.g., an AAV2 vector) provided herein can be formulated as a pharmaceutical composition for administration to a mammal (e.g. a human or a non-human primate). In some cases, a pharmaceutical composition provided herein can include a pharmaceutically acceptable carrier such as a buffer, a salt, a surfactant, a sugar, a tonicity modifier, or combinations thereof as, for example, described elsewhere (Gervasi et al., Eur. J. Pharmaceutics and Biopharmaceutics, 131:8-24 (2018)). Examples of pharmaceutically acceptable carriers that can be used to make a pharmaceutical composition provided herein include, without limitation, water, lactic acid, citric acid, sodium chloride, sodium citrate, sodium succinate, sodium phosphate, a surfactant (e.g., polysorbate 20, polysorbate 80, or poloxamer 188), dextran 40, or a sugar (e.g., sorbitol, mannitol, sucrose, dextrose, or trehalose), or combinations thereof. For example, a pharmaceutical composition designed to include an AAV vector (e.g., an AAV2 vector) provided herein can be formulated to include a buffer (e.g., an acetate, citrate, histidine, succinate, phosphate, or hydroxymethyl-aminomethane (Tris) buffer), a surfactant (e.g., polysorbate 20, polysorbate 80, or poloxamer 188), and a sugar such as sucrose. Other ingredients that can be included within a pharmaceutical composition provided herein include, without limitation, amino acids such as glycine or arginine, antioxidants such as ascorbic acid, methionine, or ethylenediaminetetraacetic acid (EDTA), or combinations thereof.

In some cases, when a pharmaceutical composition is formulated to include one or more AAV vectors (e.g., one or more AAV2 vectors) provided herein, any appropriate titer of the AAV vectors can be used. For example, a pharmaceutical composition provided herein can be formulated to have AAV vectors (e.g., AAV2 vectors) provided herein at a titer that is greater than 1×10⁷ (e.g., greater than 1×10⁸, greater than 1×10⁹, greater than 1×10¹⁰, greater than 1×10¹¹, greater than 1×10¹², greater than 1×10¹³, or greater than 1×10¹⁴). In some cases, a pharmaceutical composition provided herein can be formulated to have AAV vectors (e.g., AAV2 vectors) provided herein at a titer that is from about 1×10⁷ to about 1×10¹⁴ (e.g., from about 1×10⁷ to about 1×10¹³, from about 1×10⁷ to about 1×10¹², from about 1×10⁷ to about 1×10¹¹, from about 1×10⁷ to about 1×10¹⁰, from about 1×10⁸ to about 1×10¹⁴, from about 1×10⁹ to about 1×10¹⁴, from about 1×10¹⁰ to about 1×10¹⁴, from about 1×10⁸ to about 1×10¹², or from about 1×10⁹ to about 1×10¹¹).

A pharmaceutical composition provided herein can be in any appropriate form. For example, a pharmaceutical composition provided herein can be designed to be a liquid, a semi-solid, or a solid. In some cases, a pharmaceutical composition provided herein can be a liquid solution (e.g., an injectable and/or infusible solution), a dispersion, a suspension, a tablet, a pill, a powder, a microemulsion, a liposome, or a suppository. In some cases, a pharmaceutical composition provided herein can be lyophilized. In some cases, a pharmaceutical composition provided herein (e.g., a pharmaceutical composition that includes one or more AAV vectors provided herein such as one or more AAV2 vectors provided herein) can be formulated with a carrier or coating designed to protect against rapid release. For example, a pharmaceutical composition provided herein can be formulated as a controlled release formulation or as a regulated release formulation as described elsewhere (U.S. Patent Application Publication Nos. 2019/0241667; 2019/0233522; and 2019/0233498).

This document also provides nucleic acid molecules encoding an AAV capsid polypeptide that includes an amino acid sequence set forth in Table 1 (or a variant thereof) or Formula A. In some cases, a nucleic acid molecule can be designed to encode an AAV capsid polypeptide that includes an amino acid sequence that is encoded by a DNA sequence set forth in Table 1 (e.g., any one of SEQ ID NOs: 6-9).

This document also provides nucleic acid molecules encoding an AAV vector (e.g., an AAV2 vector) described herein. For example, an isolated nucleic acid molecule can be designed to encode one or more AAV vectors provided herein (e.g., an AAV having an AAV capsid polypeptide that includes an amino acid sequence set forth in Table 1 (or a variant thereof) or Formula A). In some cases, a nucleic acid molecule can be designed to encode an AAV vector having an AAV capsid polypeptide that includes an amino acid sequence that is encoded by a DNA sequence set forth in Table 1 (e.g., any one of SEQ ID NOs: 6-9).

This document also provides host cells containing a nucleic acid molecule provided herein. For example, a host cell can be designed to include a nucleic acid molecule encoding an AAV capsid polypeptide described herein and/or a nucleic acid molecule encoding an AAV vector described herein. In some cases, a host cell can be designed to include a nucleic acid molecule encoding an AAV capsid polypeptide that includes an amino acid sequence set forth in Table 1 (or a variant thereof) or Formula A. In some cases, a host cell can be designed to include a nucleic acid molecule encoding an AAV vector having an AAV capsid polypeptide that includes an amino acid sequence set forth in Table 1 (or a variant thereof) or Formula A. Examples of host cells that can be designed to include a nucleic acid molecule encoding an AAV capsid polypeptide described herein and/or a nucleic acid molecule encoding an AAV vector described herein include, without limitation, HEK293T cells (ATCC), 293AAV cells (Cell Biolabs), NEB 5-alpha cells, TakaraBio Stellar cells, and MegaX cells. Any appropriate method can be used to introduce a nucleic acid molecule provided herein (e.g., a nucleic acid molecule encoding an AAV capsid polypeptide described herein and/or an AAV vector described herein) into a cell. For example, viral transfection, electroporation, transient transfection, and gene gun techniques can be used to introduce a nucleic acid molecule provided herein into a cell.

This document also provides methods and materials for making an AAV vector (e.g., an AAV2 vector) provided herein. For example, this document provides methods and materials for making AAV vectors (e.g., AAV2 vectors) containing an AAV capsid polypeptide that includes an amino acid sequence set forth in Table 1 (or a variant thereof) or Formula A. As described herein, an AAV vector can be constructed to include an AAV capsid polypeptide that includes an amino acid sequence set forth in Table 1 (or a variant thereof) or Formula A. Any appropriate method can be used to construct an AAV vector having an AAV capsid polypeptide (e.g., an AAV2 capsid polypeptide) provided herein (e.g., a capsid polypeptide that includes an amino acid sequence set forth in Table 1 (or a variant thereof) or Formula A). For example, molecular cloning and AAV vector production techniques such as those described elsewhere can be used to construct and produce an AAV vector having an AAV capsid polypeptide (e.g., an AAV2 capsid polypeptide) provided herein (see, e.g., Sambrook et al., Molecular Cloning: A Laboratory Manual, 2nd edition, Cold Spring Harbor Laboratory, NY (1989); Ausubel et al., Current Protocols in Molecular Biology, Green Publishing Associates and John Wiley & Sons, New York, N.Y. (1994); Grieger et al., Nat. Protoc., 1 (3): 1412-28 (2006); and Flannery et al., Methods Mol. Biol., 935:351-69 (2013)). In some cases, AAV vectors can be produced in HEK293T cells (ATCC) or 293AAV cells (Cell Biolabs) using a double or triple transfection method (see, e.g., Grieger et al., Nat. Protoc., 1 (3): 1412-28 (2006); and Flannery et al., Methods Mol. Biol., 935:351-69 (2013)).

This document also provides methods and materials for using an AAV vector (e.g., an AAV2 vector) provided herein. For example, this document provides methods and materials for using AAV vectors (e.g., AAV2 vectors) containing an AAV capsid polypeptide that includes an amino acid sequence set forth in Table 1 (or a variant thereof) or Formula A. As described herein, an AAV vector provided herein can be used to infect retinal cells (e.g., retinal ganglion cells) in vivo and to deliver an exogenous nucleic acid sequence to the infected retinal cells such that the infected retinal cells express the exogenous nucleic acid sequence.

In some cases, an AAV vector (e.g., an AAV2 vector) provided herein (e.g., an AAV vector containing an AAV capsid polypeptide that includes an amino acid sequence set forth in Table 1 (or a variant thereof) or Formula A) can be used to treat a retinal condition (e.g., a retinal disease). For example, an AAV vector (e.g., an AAV2 vector) provided herein that is designed to contain and drive expression of an exogenous nucleic acid sequence encoding an RNA and/or polypeptide capable of treating a retinal condition (e.g., a retinal disease) can be administered to a mammal (e.g., a human or a non-human primate) having a retinal condition in a manner such that the AAV vector (a) infects retinal cells (e.g., retinal ganglion cells) and (b) drives expression of the delivered exogenous nucleic acid in the infected retinal cells, thereby reducing the severity of one or more symptoms of the retinal condition and/or slowing the progression of the retinal condition.

As described herein, an AAV vector (e.g., an AAV2 vector) provided herein can be designed to include and drive expression of an exogenous nucleic acid sequence encoding any appropriate RNA of interest and/or polypeptide of interest. When an AAV vector provided herein is designed to treat a retinal condition (e.g., a retinal disease), an exogenous nucleic acid sequence that encodes an RNA and/or polypeptide capable of treating the retinal condition can be included within the AAV vector. Examples of RNAs that can be encoded by an exogenous nucleic acid sequence designed to treat a retinal condition (e.g., a retinal disease) and designed to be included within an AAV vector provided herein include, without limitation, SIRNA-027 to treat, e.g., sub-foveal CNVM secondary to age-related macular degeneration (see, e.g., NCT00363714), Cand5/Bevasiranib to treat, e.g., diabetic macular edema (see, e.g., NCT00306904), PF-04523655 to treat, e.g., diabetic macular edema (see, e.g., NCT01445899), QPI-1007 to treat, e.g., optic nerve atrophy in NAION (see, e.g., NCT01064505), Aganirsen to treat, e.g., ischemic CRVO to prevent neovascular glaucoma (see, e.g., NCT02947867), QR-421a to treat, e.g., retinitis pigmentosa/Usher syndrome type 2 (see, e.g., NCT03780257), QR-1123 to treat, e.g., autosomal dominant retinitis pigmentosa (see, e.g., NCT04123626), IONIS-FB-LRx to treat, e.g., geographic atrophy secondary to age-related macular degeneration (see, e.g., NCT03815825), and Sepofarsen/QR-110 to treat, e.g., Leber's congenital amaurosis (see, e.g., NCT03913143). Examples of polypeptides that can be encoded by an exogenous nucleic acid sequence designed to treat a retinal condition (e.g., a retinal disease) and designed to be included within an AAV vector provided herein include, without limitation, an ABCA4 polypeptide, a CRB1 polypeptide, an NPHP5 polypeptide, an NR2E3 polypeptide, a PDE6A polypeptide, a PDE6B polypeptide, a PDE6C polypeptide, a PRPF31 polypeptide, a RPE65 polypeptide, a RPGR polypeptide, a RS1 polypeptide, a TYR polypeptide, a USH2A polypeptide, a MYO7A polypeptide, an REP1 polypeptide, an OPN1LW polypeptide, an OPN1MW polypeptide, a CNGA3 polypeptide, a CNGB3 polypeptide, a GUCY2D polypeptide, a GACA1A polypeptide, a GNAT2 polypeptide, a PDE6H polypeptide, a PROM1 polypeptide, a PRPH2 polypeptide, a CRX polypeptide, an NPHP5 polypeptide, an EYS polypeptide, an ND4 polypeptide, a CLN1-14 polypeptide (e.g., a CLN3 polypeptide, a CLN5 polypeptide, a CLN6 polypeptide, or a CLN8 polypeptide), an NYX polypeptide, a GRM6 polypeptide, a TRPM1 polypeptide, a GPR179 polypeptide, an LRIT3 polypeptide, a glial cell derived neurotrophic factor (GDNF) polypeptide, a brain-derived neurotrophic factor (BDNF) polypeptide, a fibroblast growth factor (FGF) polypeptide, a truncated rod-derived cone viability factor (RdCVF) polypeptide, a full-length rod-derived cone viability factor (RdCVFL) polypeptide, an X-linked inhibitor of apoptosis (XIAP) polypeptide, a soluble fins-related receptor tyrosine kinase 1 (sFLT) polypeptide, a CYP4V2 polypeptide, a palmitoyl protein thioesterase 1 polypeptide, a tripeptidyl peptidase 1 polypeptide, a DNAJC5 polypeptide, a MFSD8 polypeptide, a cathepsin D polypeptide, a granulin polypeptide, an ATP13A2 polypeptide, a cathepsin F polypeptide, a KCTD7 polypeptide, a “P” gene polypeptide, a TRP1 polypeptide, a MATP (SLC45A2) polypeptide, a SLC24A5 polypeptide, a LRMDA polypeptide, a GPR143 polypeptide, an RPGR-exon 1-ORF15 polypeptide, an USH2b polypeptide, an USH1C polypeptide, a CDH23 polypeptide, a PCDH15 polypeptide, a SANS polypeptide, an USH1H polypeptide, a CIB2 polypeptide, an USH1K polypeptide, an ADGRV1 polypeptide, a WHRN polypeptide, a PDZD7 polypeptide, a CLRN1 polypeptide, a HARS polypeptide, an RP2 polypeptide, a FAM161 polypeptide, a DLK polypeptide, a RHO polypeptide, a CHM polypeptide, a BEST1 polypeptide, a RPI polypeptide, an OPA1 polypeptide, a CEP290 polypeptide, a RDH12 polypeptide, a CACNA1F polypeptide, a BBS1 polypeptide, a FAM161A polypeptide, a CERKL polypeptide, a PRPF8 polypeptide, a RP1L1 polypeptide, a SNRNP200 polypeptide, an IMPG2 polypeptide, a CDHR1 polypeptide, an IMPDH1 polypeptide, a CNGB1 polypeptide, a MERTK polypeptide, a KCNV2 polypeptide, an A1PL1 polypeptide, a RPGRIP1 polypeptide, a TULP1 polypeptide, a C2ORF71 (aka PCARE) polypeptide, a MAK polypeptide, a TIMP3 polypeptide, a GUCA1A polypeptide, an ALMS1 polypeptide, a BBS10 polypeptide, an IFT140 polypeptide, a CNGA1 polypeptide, a NMNAT1 polypeptide, a COL2A1 polypeptide, an EFEMP1 polypeptide, a WFS1 polypeptide, a RDH5 polypeptide, a PRPF3 polypeptide, a LRP5 polypeptide, a TOPORS polypeptide, a DHDDS polypeptide, a LCA5 polypeptide, an IQCB1 polypeptide, a RP9 polypeptide, an ATXN7 polypeptide, a BBS2 polypeptide, a SAG RLBP1 polypeptide, a ND6 (MT-ND6) polypeptide, a C1QTNF5 polypeptide, a VPS13B polypeptide, a KIF11 polypeptide, a MT-TL1 polypeptide, a KLHL7 polypeptide, an ACO2 polypeptide, a C21orf2 (aka CFAP410) polypeptide, an AHI1 polypeptide, a KIZ polypeptide, a SPATA7 polypeptide, a TTLL5 polypeptide, an HGSNAT polypeptide, a NRL polypeptide, an OAT polypeptide, a FLVCR1 polypeptide, an ABCC6 polypeptide, a LRAT polypeptide, a CEP78 polypeptide, a CDH3 polypeptide, a FZD4 polypeptide, a BBS12 polypeptide, an HK1 polypeptide, a PRDM13 polypeptide, an ADAM9 polypeptide, a BBS7 polypeptide, a CABP4 polypeptide, an ABHD12 polypeptide, a COL18A1 polypeptide, a MFRP polypeptide, a RIMS1 polypeptide, a ROMI polypeptide, a BBS4 polypeptide, an IMPG1 polypeptide, an INPP5E polypeptide, a VCAN polypeptide, a POC1B polypeptide, a RAX2 polypeptide, a TSPAN12 polypeptide, a CACNA2D4 polypeptide, a JAG1 polypeptide, a MKKS polypeptide, a NPHP4 polypeptide, a BBS9 polypeptide, a COL11A1 polypeptide, an ELOVL4 polypeptide, a NDP polypeptide, a NPHP1 polypeptide, a RGR polypeptide, a BBS5 polypeptide, a WDR19 polypeptide, a C8ORF37 polypeptide, a CTNNA1 polypeptide, a LAMP2 polypeptide, a PEX1 polypeptide, a PHYH polypeptide, an ATF6 polypeptide, a PRPS1 polypeptide, a SEMA4A polypeptide, an ARL6 polypeptide, a CNNM4 polypeptide, an OTX2 polypeptide, a PRPF6 polypeptide, a RBP3 polypeptide, a PNPLA6 polypeptide, a SLC24A1 polypeptide, an USH1G polypeptide, a PITPNM3 polypeptide, a TTC8 polypeptide, an ARSG polypeptide, a CWC27 polypeptide, a DRAM2 polypeptide, a PRCD polypeptide, a REEP6 polypeptide, a SSBP1 polypeptide, a LAMAI polypeptide, a RAB28 polypeptide, a ZNF408 polypeptide, a GNAT1 polypeptide, an IDH3A polypeptide, a PDE6G polypeptide, a PEX6 polypeptide, a TUB polypeptide, a CEP250 polypeptide, a FSCN2 polypeptide, a GRK1 polypeptide, a RBP4 polypeptide, a RD3 polypeptide, an AGBL5 polypeptide, a CAPN5 polypeptide, an IFT172 polypeptide, a KCNJ13 polypeptide, a PAX2 polypeptide, a CC2D2A polypeptide, a HMCN1 polypeptide, a MT-ATP6 polypeptide, a RCBTB1 polypeptide, an ARL2BP polypeptide, a CA4 polypeptide, a DFNB31 polypeptide, a GNB3 polypeptide, a MMACHC polypeptide, a PRPF4 polypeptide, a RGS9 polypeptide, an ARHGEF18 polypeptide, a KIAA1549 polypeptide, a MKS1 polypeptide, a MTTP (not MT-TP) polypeptide, a PLK4 polypeptide, a RPGRIP1L polypeptide, a SDCCAG8 polypeptide, a SRD5A3 polypeptide, a TUBB4B polypeptide, an ADAMTS18 polypeptide, an ARL3 polypeptide, a COL11A2 polypeptide, a MVK polypeptide, a NBAS polypeptide, an OFD1 polypeptide, a P3H2 polypeptide, a RGS9BP polypeptide, a CSPP1 polypeptide, an ITM2B polypeptide, a PANK2 polypeptide, a PEX7 polypeptide, a POMGNT1 polypeptide, a SLC4A7 polypeptide, a TMEM231 polypeptide, a TRNT1 polypeptide, a TUBGCP6 polypeptide, a ZNF513 polypeptide, an AFG3L2 polypeptide, an ARL13B polypeptide, a C5ORF42 (aka CPLANE1) polypeptide, a COL9A1 polypeptide, a CTSD polypeptide, a DTHD1 polypeptide, a DYNC2HI polypeptide, an IFT81 polypeptide, a KIAA0586 polypeptide, a MFN2 polypeptide, a NPHP3 polypeptide, a PCYT1A polypeptide, a PEX12 polypeptide, a PLA2G5 polypeptide, a POC5 polypeptide, a SCAPER polypeptide, a SLC25A46 polypeptide, a TMEM237 polypeptide, a TRAF3IP1 polypeptide, a TTC21B polypeptide, a TUBGCP4 polypeptide, an ADIPOR1 polypeptide, a CEP164 polypeptide, a CLCC1 polypeptide, a COL9A2 polypeptide, a CTNNB1 polypeptide, a DHX38 polypeptide, a GNPTG polypeptide, a GRN polypeptide, a GUCA1B polypeptide, an IFT27 polypeptide, an IFT74 polypeptide, a KIAA0556 polypeptide, a LRP2 polypeptide, a MAPKAPK3 polypeptide, a MIR204 polypeptide, a MT-ND3 polypeptide, a MT-RNR1 polypeptide, a MT-TS2 polypeptide, a ND5 (MT-ND5) polypeptide, a NEK2 polypeptide, an OPN1SW polypeptide, a PEX13 polypeptide, a PEX2 polypeptide, a RHBDD2 polypeptide, a SAMD11 polypeptide, a SCLT1 polypeptide, a SLC7A14 polypeptide, a TCTN1 polypeptide, a TCTN2 polypeptide, a TLCD3B polypeptide, a TREX1 polypeptide, a TTPA polypeptide, an UNCI19 polypeptide, a WDPCP polypeptide, an ACBD5 polypeptide, an AHR polypeptide, an ARMC9 polypeptide, an ASRGL1 polypeptide, an ATOH7 polypeptide, a B9DI polypeptide, a B9D2 polypeptide, a BBIP1 polypeptide, a C12ORF65 polypeptide, a C2CD3 polypeptide, a C5AR2 polypeptide, a CCDC188 polypeptide, a CCT2 polypeptide, a CEP104 polypeptide, a CEP120 polypeptide, a CEP19 polypeptide, a CEP41 polypeptide, a CISD2 polypeptide, a CLUAP1 polypeptide, a COL9A3 polypeptide, a CRB2 polypeptide, a CTC1 polypeptide, a DACT2 polypeptide, a DDR1 polypeptide, an ENSA polypeptide, an ESPN polypeptide, an EXOSC2 polypeptide, a FBN3 polypeptide, a GDF6 polypeptide, a GPR125 polypeptide, a HKDC1 polypeptide, a HMX1 polypeptide, an IDH3B polypeptide, an IFT43 polypeptide, an IFT80 polypeptide, an INVS polypeptide, a KIAA0753 polypeptide, a KIF3B polypeptide, a KIF7 polypeptide, a LRRTM4 polypeptide, a LZTFL1 polypeptide, a MT-ATP8 polypeptide, a MT-CO1 polypeptide, a MT-CO2 polypeptide, a MT-CO3 polypeptide, a MT-CYB polypeptide, a MT-ND2 polypeptide, a MT-ND4L polypeptide, a MT-RNR2 polypeptide, a MT-TA polypeptide, a MT-TC polypeptide, a MT-TD polypeptide, a MT-TE polypeptide, a MT-TF polypeptide, a MT-TG polypeptide, a MT-TH polypeptide, a MT-TI polypeptide, a MT-TK polypeptide, a MT-TL2 polypeptide, a MT-TM polypeptide, a MT-TN polypeptide, a MT-TP (Not MTTP) polypeptide, a MT-TQ polypeptide, a MT-TR polypeptide, a MT-TS1 polypeptide, a MT-TT polypeptide, a MT-TV polypeptide, a MT-TW polypeptide, a MT-TY polypeptide, a NEUROD1 polypeptide, a PDE6D polypeptide, a PEX10 polypeptide, a PEX11B polypeptide, a PEX14 polypeptide, a PEX16 polypeptide, a PEX19 polypeptide, a PEX26 polypeptide, a PEX3 polypeptide, a PEX5 polypeptide, a PGK1 polypeptide, a PISD polypeptide, a PPP2R3C polypeptide, a PROS1 polypeptide, a PSEN1 polypeptide, a RDH11 polypeptide, a RRM2B polypeptide, a SMARCA4 polypeptide, a SPP2 polypeptide, a TCTN3 polypeptide, a TEAD1 polypeptide, a TMEM107 polypeptide, a TMEM138 polypeptide, a TMEM216 polypeptide, a TMEM67 polypeptide, a TPP1 polypeptide, a TRIM32 polypeptide, a USP45 polypeptide, and a ZNF423 polypeptide.

Any appropriate retinal condition (e.g., a retinal disease) can be treated using an AAV vector (e.g., an AAV2 vector) provided herein (e.g., an AAV vector containing an AAV capsid polypeptide that includes an amino acid sequence set forth in Table I (or a variant thereof) or Formula A and an exogenous nucleic acid sequence encoding a therapeutic RNA and/or polypeptide). Examples of such retinal conditions include, without limitation, Leber congenital amaurosis (LCA), Leber hereditary optic neuropathy (LHON), oculocutaneous albinism type 1 (OCA1), retinitis pigmentosa, rod/cone dystrophy, cone dystrophy, rod dystrophy, Stargardt Disease, Usher syndrome, X-linked retinitis pigmentosa (XLRP), X-linked retinoschisis (XLRS), choroideremia, achromatopsia, blue cone monochromacy, color blindness, glaucoma, optic atrophy, Batten disease, congenital stationary night blindness (CSNB), macular degeneration, CRB1-related retinal dystrophy, and foveal cone dystrophy.

Examples of therapeutic RNAs and polypeptides that can be delivered using an AAV vector provided herein to treat particular retinal conditions are set forth in Tables 2 and 3. Examples of genomic nucleic acids that can be inactivated and/or knocked out to treat particular retinal conditions using one or more AAV vectors provided herein that are designed to deliver gene editing components are set forth in Table 3. Examples of genomic nucleic acids of disease causing alleles that can be replaced with healthy alleles to treat particular retinal conditions using one or more AAV vectors provided herein that are designed to deliver gene editing components are set forth in Table 3.

TABLE 2
Examples of therapeutic polypeptide for treating retinal conditions.

| Retinal Condition | Examples of therapeutic polypeptides |
|---|---|
| Stargardt Disease | ABCA4 |
| Leber congenital amaurosis 8 | CRB1 |
| Leber congenital amaurosis | NPHP5 |
| Retinitis pigmentosa 37 | NR2E3 |
| Rod/Cone dystrophy | PDE6A/PDE6B |
| Rod/Cone dystrophy | PDE6c |
| Retinitis pigmentosa 11 | PRPF31 |
| Leber congenital amaurosis 2 | RPE65 |
| X-linked retinitis pigmentosa | RPGR |
| X-linked retinoschisis | RS1 |
| Oculocutaneous albinism type 1 | TYR |
| Usher syndrome | PCDH15 |
| Usher syndrome | USH2a |
| Usher syndrome | USH2b |
| Usher syndrome, subtype IB caused by | MYO7A |
| mutations in the MYO7A gene | |
| Usher syndrome, subtype IC caused by | USH1C |
| mutations in the USH1C gene | |
| Usher syndrome, subtype ID caused by | CDH23 |
| mutations in the CDH23 gene | |
| Usher syndrome, subtype ID-F caused by | PCDH15 and/or CDH23 |
| mutations in the PCDH15 and/or CDH23 | |
| genes | |
| Usher syndrome, subtype IF caused by | PCDH15 |
| mutations in the PCDH15 gene | |
| Usher syndrome, subtype IG caused by | SANS |
| mutations in the SANS gene | |
| Usher syndrome, subtype IH caused by | USH1H |
| mutations in the USH1H gene | |
| Usher syndrome, subtype IJ caused by | CIB2 |
| mutations in the CIB2 gene | |
| Usher syndrome, subtype IK caused by | USH1K |
| mutations in the USH1K gene | |
| Usher syndrome, subtype IIA, caused by | USH2A |
| mutations in the USH2A | |
| Usher syndrome, subtype IIC caused by | ADGRV1 |
| mutations in the ADGRV1 gene | |
| Usher syndrome, subtype IID caused by | WHRN |
| mutations in the WHRN gene | |
| Usher syndrome, subtype IIC caused by | GPR98 and/or PDZD7 |
| mutations in the GPR98 and/or PDZD7 | |
| genes (ADGRV1 is also known as GPR98) | |
| Usher syndrome, subtype IIIA caused by | CLRN1 |
| mutations in the CLRN1 gene | |
| Usher syndrome, subtype IIIB caused by | IIIB, caused by mutations in the HARS |
| mutations in the HARS gene | gene |
| Retinitis pigmentosa | One or more trophic factors |
| Leber congenital amaurosis | One or more trophic factors |
| Achromatopsia | CNGA3, CNGB3, and/or PDE6H |
| Any blinding disease | Optogenetic tools: Chr, NhpR, ReachR, |
| | and/or others |
| Blue cone monochromacy | OPN1LW and/or OPN1MW |
| Cone dystrophy | GNAT2 |
| Cone-rod dystrophy | PDE6C and/or PROM1 |

TABLE 3
Examples of polypeptide that can be expressed to treat retinal conditions, examples
of polypeptides that can be knocked out to treat retinal conditions, and/or examples
of polypeptides that can be knocked out and replace with an alternative (e.g.,
wild-type or non-disease version) to treat retinal conditions.

| | | | | Gene |
|---|---|---|---|---|
| | | Gene | Gene | editing- |
| Disease | Polypeptide | expression | KO | replacement |
| Achromatopsia | CNGA3 | X | | |
| Achromatopsia | CNGB3 | X | | |
| Achromatopsia | PDE6H | X | | |
| Any blinding disease | Optogenetic | X | | |
| | tools: Chr, | | | |
| | NhpR, and/or | | | |
| | ReachR | | | |
| Batten disease | CTSD | X | | |
| Bietti crystalline dystrophy | CYP4V2 | X | | X |
| Blue cone monochromacy | OPN1LW | X | | |
| Blue cone monochromacy | OPN1MW | X | | |
| Choroideremia | REP1 | X | | |
| Cone dystrophy | PDE6c | X | | |
| Cone dystrophy | GNAT2 | X | | |
| Cone-rod dystrophy | PDE6C | X | | |
| Cone-rod dystrophy | PROM1 | X | | |
| Cone/rod dystrophy | PRPH2 | X | X | X |
| CSNB | NYX | X | | |
| CSNB | GRM6 | X | | |
| CSNB | TRPM1 | X | | |
| CSNB | GPR179 | X | | |
| CSNB | LR1T3 | X | | |
| Glaucoma | Trophic factors | X | | |
| Glaucoma | Complement | X | | |
| | inhibition | | | |
| | factors | | | |
| Glaucoma | Apoptosis | X | | |
| | inhibition | | | |
| | factors | | | |
| Glaucoma | Survival factors | X | | |
| Glaucoma | Neuroprotective | X | | |
| | factors | | | |
| LCA | NPHP5 | X | | |
| LCA | GUCY2D | X | | |
| Leber congenital amaurosis | RPE65 | X | | |
| Leber congenital amaurosis 8 | CRB1 | X | | X |
| LHON | ND4 | X | | |
| Macular dystrophy | CRX | X | X | X |
| Oculocutaneous albinism | TYR | X | | |
| type 1 | | | | |
| Optic atrophy | CLN1-14 | X | | |
| Retinitis pigmentosa | EYS | X | | X |
| Retinitis pigmentosa | RHO | X | X | X |
| Retinitis pigmentosa | PRPF31 | X | | |
| Retinitis pigmentosa | PDE6A | X | | |
| Bestrophinopathy | BEST1 | X | X | |
| Retinitis pigmentosa | PRPF3 | X | | |
| Retinitis pigmentosa | PRPF8 | X | | |
| Retinitis pigmentosa | TOPORS | X | X | X |
| Retinitis pigmentosa 37 | NR2E3 | X | X | X |
| Rod/Cone dystrophy | PDE6B | X | | |
| RP, LCA, Others | Trophic factors | X | | |
| Stargardt Disease | ABCA4 | X | | X |
| Usher syndrome | PCDH15 | X | | |
| Usher syndrome | USH2A | X | | X |
| Usher syndrome | MYO7A | X | | X |
| XLRP | RPGR | X | | |
| XLRS | RS1 | X | | |
| Wet AMD | Survival factors | X | | |
| Dry AMD | Survival factors | X | | |
| Diabetic retinopathy | Survival factors | X | | |
| Diabetic Macular Edema | Survival factors | X | | |
| Retinitis pigmentosa | Survival factors | X | | |
| Wet AMD | Apoptosis | X | | |
| | inhibition | | | |
| | factors | | | |
| Dry AMD | Apoptosis | X | | |
| | inhibition | | | |
| | factors | | | |
| Diabetic retinopathy | Apoptosis | X | | |
| | inhibition | | | |
| | factors | | | |
| Diabetic Macular Edema | Apoptosis | X | | |
| | inhibition | | | |
| | factors | | | |
| Retinitis pigmentosa | Apoptosis | X | | |
| | inhibition | | | |
| | factors | | | |
| Wet AMD | Complement | X | | |
| | inhibition | | | |
| | factors | | | |
| Dry AMD | Complement | X | | |
| | inhibition | | | |
| | factors | | | |
| Diabetic retinopathy | Complement | X | | |
| | inhibition | | | |
| | factors | | | |
| Diabetic Macular Edema | Complement | X | | |
| | inhibition | | | |
| | factors | | | |
| Wet AMD | Neuroprotective | X | | |
| | factors | | | |
| Dry AMD | Neuroprotective | X | | |
| | factors | | | |
| Diabetic retinopathy | Neuroprotective | X | | |
| | factors | | | |
| Diabetic Macular Edema | Neuroprotective | X | | |
| | factors | | | |
| Retinitis pigmentosa | Neuroprotective | X | | |
| | factors | | | |
| Wet AMD | Anti-VEGF | X | | |
| | polypeptides | | | |
| Diabetic retinopathy | Anti-VEGF | | | |
| | polypeptides | | | |
| Diabetic Macular Edema | Anti-VEGF | X | | |
| | polypeptides | | | |
| Wet AMD | Optogenetic | X | | |
| | tools: Chr, | | | |
| | NhpR, and/or | | | |
| | ReachR | | | |
| Dry AMD | Optogenetic | X | | |
| | tools: Chr, | | | |
| | NhpR, and/or | | | |
| | ReachR | | | |
| Diabetic retinopathy | Optogenetic | X | | |
| | tools: Chr, | | | |
| | NhpR, and/or | | | |
| | ReachR | | | |
| Diabetic Macular Edema | Optogenetic | X | | |
| | tools: Chr, | | | |
| | NhpR, and/or | | | |
| | ReachR | | | |
| Retinitis pigmentosa | Optogenetic | X | | |
| | tools: Chr, | | | |
| | NhpR, and/or | | | |
| | ReachR | | | |

In some cases, a retinal condition can be treated using an AAV vector provided herein that is designed to express one or more polypeptides having the ability to inhibit vascular angiogenesis. Examples of polypeptides having the ability to inhibit vascular angiogenesis that can be used as described herein include, without limitation, monoclonal anti-VEGF antibody polypeptides, angiostatin polypeptides, siRNA polypeptides, and endostatin polypeptides. In some cases, wet AMD can be treated using an AAV vector provided herein that is designed to express a monoclonal anti-VEGF antibody polypeptide, an angiostatin polypeptide, an siRNA, and/or endostatin polypeptide. In some cases, diabetic retinopathy can be treated using an AAV vector provided herein that is designed to express a monoclonal anti-VEGF antibody polypeptide, an angiostatin polypeptide, an siRNA, and/or an endostatin polypeptide. In some cases, diabetic macular edema can be treated using an AAV vector provided herein that is designed to express a monoclonal anti-VEGF antibody polypeptide, an angiostatin polypeptide, an siRNA, and/or an endostatin polypeptide.

In some cases, a retinal condition can be treated using an AAV vector provided herein that is designed to express one or more polypeptides with neuroprotective capabilities. Examples of polypeptides having the ability to provide neuroprotective activity that can be used as described herein include, without limitation, GDNF polypeptides, CNTF polypeptides, IGF-1 polypeptides, VEGF polypeptides, and BDNF polypeptides. In some cases, wet AMD can be treated using an AAV vector provided herein that is designed to express a GDNF polypeptide, a CNTF polypeptide, an IGF-1 polypeptide, a VEGF polypeptide, and/or a BDNF polypeptide. In some cases, dry AMD can be treated using an AAV vector provided herein that is designed to express a GDNF polypeptide, a CNTF polypeptide, an IGF-1 polypeptide, a VEGF polypeptide, and/or a BDNF polypeptide. In some cases, diabetic retinopathy can be treated using an AAV vector provided herein that is designed to express a GDNF polypeptide, a CNTF polypeptide, an IGF-1 polypeptide, a VEGF polypeptide, and/or a BDNF polypeptide. In some cases, diabetic macular edema can be treated using an AAV vector provided herein that is designed to express a GDNF polypeptide, a CNTF polypeptide, an IGF-1 polypeptide, a VEGF polypeptide, and/or a BDNF polypeptide.

In some cases, a retinal condition can be treated using an AAV vector provided herein that is designed to express one or more polypeptides having the ability to provide optogenetic capabilities. Examples of polypeptides having the ability to provide optogenetic capabilities that can be used as described herein include, without limitation, ChR polypeptides, ChR2 polypeptides, ArchT polypeptides, NpHR polypeptides, and ChrimsonR polypeptides. In some cases, wet AMD can be treated using an AAV vector provided herein that is designed to express a ChR polypeptide, a ChR2 polypeptide, an ArchT polypeptide, a NpHR polypeptide, and/or a ChrimsonR polypeptide. In some cases, dry AMD can be treated using an AAV vector provided herein that is designed to express a ChR polypeptide, a ChR2 polypeptide, an ArchT polypeptide, a NpHR polypeptide, and/or a ChrimsonR polypeptide. In some cases, diabetic retinopathy can be treated using an AAV vector provided herein that is designed to express a ChR polypeptide, a ChR2 polypeptide, an ArchT polypeptide, a NpHR polypeptide, and/or a ChrimsonR polypeptide. In some cases, diabetic macular edema can be treated using an AAV vector provided herein that is designed to express a ChR polypeptide, a ChR2 polypeptide, an ArchT polypeptide, a NpHR polypeptide, and/or a ChrimsonR polypeptide.

In some cases, a retinal condition can be treated using an AAV vector provided herein that is designed to express one or more polypeptides having the ability to inhibit apoptosis. Examples of polypeptides having the ability to inhibit apoptosis that can be used as described herein include, without limitation, XIAP polypeptides, cIAP1 polypeptides, C-IAP2 polypeptides, Livin polypeptides, and Survivin polypeptides. In some cases, wet AMD can be treated using an AAV vector provided herein that is designed to express a XIAP polypeptide, a cIAP1 polypeptide, a C-IAP2 polypeptide, a Livin polypeptide, and/or a Survivin polypeptide. In some cases, diabetic retinopathy can be treated using an AAV vector provided herein that is designed to express a XIAP polypeptide, a cIAP1 polypeptide, a C-IAP2 polypeptide, a Livin polypeptide, and/or a Survivin polypeptide. In some cases, diabetic macular edema can be treated using an AAV vector provided herein that is designed to express a XIAP polypeptide, a cIAP1 polypeptide, a C-IAP2 polypeptide, a Livin polypeptide, and/or a Survivin polypeptide.

In some cases, a retinal condition can be treated using an AAV vector provided herein that is designed to express one or more polypeptides having the ability to inhibit complement. Examples of polypeptides having the ability to inhibit complement that can be used as described herein include, without limitation, Complement Factor I polypeptides, Complement factor H polypeptides, and sCD59 polypeptides. In some cases, wet AMD can be treated using an AAV vector provided herein that is designed to express a Complement Factor I polypeptide, a Complement factor H polypeptide, and/or a sCD59 polypeptide. In some cases, dry AMD can be treated using an AAV vector provided herein that is designed to express a Complement Factor I polypeptide, a Complement factor H polypeptide, and/or a sCD59 polypeptide. In some cases, diabetic retinopathy can be treated using an AAV vector provided herein that is designed to express a Complement Factor I polypeptide, a Complement factor H polypeptide, and/or a sCD59 polypeptide. In some cases, diabetic macular edema can be treated using an AAV vector provided herein that is designed to express a Complement Factor I polypeptide, a Complement factor H polypeptide, and/or a sCD59 polypeptide.

In some cases, a retinal condition can be treated using an AAV vector provided herein that is designed to express one or more polypeptides having the ability to induce survival factors. Examples of polypeptides having the ability to induce survival factors that can be used as described herein include, without limitation, RdCVF polypeptides, RdCVFL polypeptides, HIF-1 polypeptides, IAP family polypeptides, and BCL-2 family polypeptides. In some cases, wet AMD can be treated using an AAV vector provided herein that is designed to express a RdCVF polypeptide, a RdCVFL polypeptide, an HIF-1 polypeptide, an IAP family polypeptide, and/or a BCL-2 family polypeptide. In some cases, dry AMD can be treated using an AAV vector provided herein that is designed to express a RdCVF polypeptide, a RdCVFL polypeptide, an HIF-1 polypeptide, an IAP family polypeptide, and/or a BCL-2 family polypeptide. In some cases, diabetic retinopathy can be treated using an AAV vector provided herein that is designed to express a RdCVF polypeptide, a RdCVFL polypeptide, an HIF-1 polypeptide, an IAP family polypeptide, and/or a BCL-2 family polypeptide. In some cases, diabetic macular edema can be treated using an AAV vector provided herein that is designed to express a RdCVF polypeptide, a RdCVFL polypeptide, an HIF-1 polypeptide, an IAP family polypeptide, and/or a BCL-2 family polypeptide.

Any appropriate method can be used to administer an AAV vector provided herein or composition (e.g., a pharmaceutical composition) provided herein to a mammal (e.g., a human or a non-human primate). For example, a composition provided herein (e.g., a pharmaceutical composition containing one or more AAV vectors provided herein) can be administered to a mammal (e.g., a human or a non-human primate) intravitreally, intravenously (e.g., via an intravenous injection or infusion), subcutaneously (e.g., via a subcutaneous injection), intraperitoneally (e.g., via an intraperitoneal injection), orally, via inhalation, intramuscularly (e.g., via intramuscular injection), subretinally, intravitreally, systemically, or suprachoroidally. In some cases, the route and/or mode of administration of a composition (e.g., a pharmaceutical composition provided herein) can be adjusted for the mammal being treated.

In some cases, an effective amount of a composition containing an AAV vector provided herein (e.g., a pharmaceutical composition provided herein) to treat a retinal condition can be an amount that reduces the severity of one or more symptoms of the retinal condition and/or slows the progression of the retinal condition without producing significant toxicity to the mammal. For example, an effective amount of an AAV vector provided herein can be from about 1×10⁷ viral genomes to about 1×10¹⁴ viral genomes (e.g., from about 1×10⁷ viral genomes to about 1×10¹³ viral genomes, from about 1×10⁷ viral genomes to about 1×10¹² viral genomes, from about 1×10⁷ viral genomes to about 1×10¹¹ viral genomes, from about 1×10⁷ viral genomes to about 1×10¹⁰ viral genomes, from about 1×10⁸ viral genomes to about 1×10¹⁴ viral genomes, from about 1×10⁹ viral genomes to about 1×10¹⁴ viral genomes, from about 1×10¹⁰ viral genomes to about 1×10¹⁴ viral genomes, from about 1×10⁸ viral genomes to about 1×10¹² viral genomes, or from about 1×10⁹ viral genomes to about 1×10¹¹ viral genomes). In some cases, an effective amount of an AAV vector provided herein can be from about 1×10¹⁰ viral genomes/kg of body weight to about 1×10¹⁴ viral genomes/kg of body weight (e.g., from about 1×10¹⁰ viral genomes/kg of body weight to about 1×10¹³ viral genomes/kg of body weight, from about 1×10¹⁰ viral genomes/kg of body weight to about 1×10¹² viral genomes/kg of body weight, from about 1×10¹⁰ viral genomes/kg of body weight to about 1×10¹¹ viral genomes/kg of body weight). The effective amount can remain constant or can be adjusted as a sliding scale or variable dose depending on the mammal's response to treatment. Various factors can influence the actual effective amount used for a particular application. For example, the severity of a retinal condition, the route of administration, the age and general health condition of the mammal, excipient usage, the possibility of co-usage with other therapeutic or prophylactic treatments such as use of other retinal drugs, and the judgment of the treating physician may require an increase or decrease in the actual effective amount of a composition provided herein (e.g., a pharmaceutical composition containing an AAV vector provided herein) that is administered.

In some cases, an effective frequency of administration of a composition containing an AAV vector provided herein (e.g., a pharmaceutical composition provided herein) can be a frequency that reduces the severity of one or more symptoms of the retinal condition and/or slows the progression of the retinal condition without producing significant toxicity to the mammal. Various factors can influence the actual effective frequency used for a particular application. For example, the severity of a retinal condition, the route of administration, the age and general health condition of the mammal, excipient usage, the possibility of co-usage with other therapeutic or prophylactic treatments such as use of other retinal drugs, and the judgment of the treating physician may require an increase or decrease in the actual effective frequency of administration of a composition provided herein.

In some cases, an effective duration of administration of a composition containing an AAV vector provided herein (e.g., a pharmaceutical composition provided herein) can be a duration that reduces the severity of one or more symptoms of the retinal condition and/or slows the progression of the retinal condition without producing significant toxicity to the mammal. For example, an effective duration of administration of a pharmaceutical composition provided herein can vary from a single time point of administration to several weeks to several months (e.g., 4 to 12 weeks). In some cases, the duration can be for as long as the mammal is alive. Multiple factors can influence the actual effective duration used for a particular application. For example, the severity of a retinal condition, the route of administration, the age and general health condition of the mammal, excipient usage, the possibility of co-usage with other therapeutic or prophylactic treatments such as use of other retinal drugs, and the judgment of the treating physician may require an increase or decrease in the actual effective duration of administration of a composition provided herein (e.g., a pharmaceutical composition containing an AAV vector provided herein).

In some cases, an effective amount of a composition containing an AAV vector provided herein (e.g., a pharmaceutical composition provided herein) to treat a retinal condition can be administered once or twice to a mammal (e.g., a human or a non-human primate) to treat that mammal.

The invention will be further described in the following examples, which do not limit the scope of the invention described in the claims.

### EXAMPLES

### Example 1—Construction of AAV Vectors Containing Mutated Capsid Polypeptides

A high-throughput method was used to create AAV vectors with mutated capsid polypeptides and to screen those created AAV vectors for particular AAV vectors having the ability to exhibit high efficiency and/or specificity for infecting retinal cells. See, e.g., Öztürk et al., bioRxiv, 2020.10.01.323196 (2020) and Öztürk et al., eLife, 10: e64175 (2021). Briefly, highly complex libraries of AAV mutants were created and injected into the eyes of primates (cynolmolgus macaques or rhesus macaques). These libraries were created such that each AAV vector in the library contained a unique DNA barcode, which allowed for tracking of a mutated AAV capsid polypeptide. In one library version, successfully packaged AAV vectors were polymerase chain reaction (PCR) amplified and repackaged, resulting in a “repack” library. In another library version, AAV vectors were injected into primate retinas, and nucleic acid encoding the AAV capsid polypeptides were then amplified from the nuclei of foveal cells, resulting in an “enriched” library. Each iteration of the AAV library (e.g., the original library, the repack library, and the enriched library) was injected intravitreally into primate eyes.

After injection, the AAV vectors competed with each other in vivo. Infection of successful AAV vectors led to expression of the DNA barcodes. Single cell suspensions were created from isolated retinal tissue, and single cell microfluidic technology (10× Genomics) was used to create cDNA libraries of individual cells. Computational analysis was performed to identify optimal vectors, according to cell specificities, expression levels, and/or other desirable characteristics, based on the presence and quantity of DNA barcodes in transcriptomes from thousands of different cells of multiple cell types in parallel. The performance of AAV capsid polypeptides was evaluated on the basis of mRNA transcription levels rather than the presence of DNA, reflecting the ability of the AAV vectors to drive expression of the AAV vector nucleic acid as opposed to simply having the ability to enter a cell.

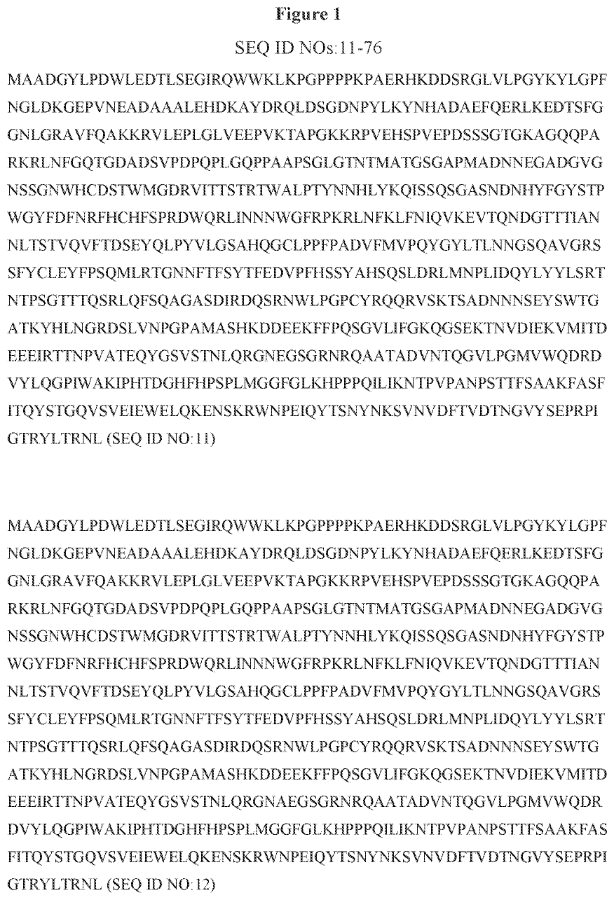

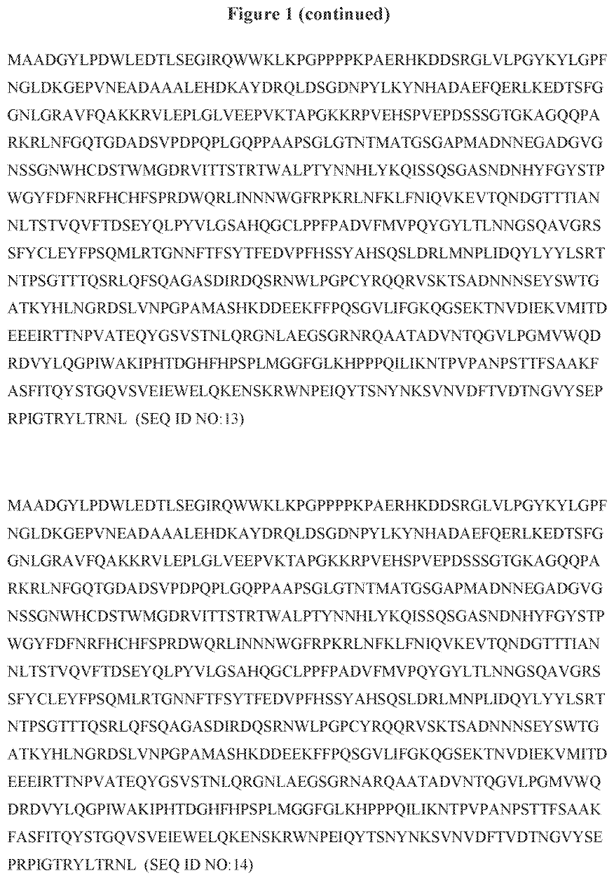

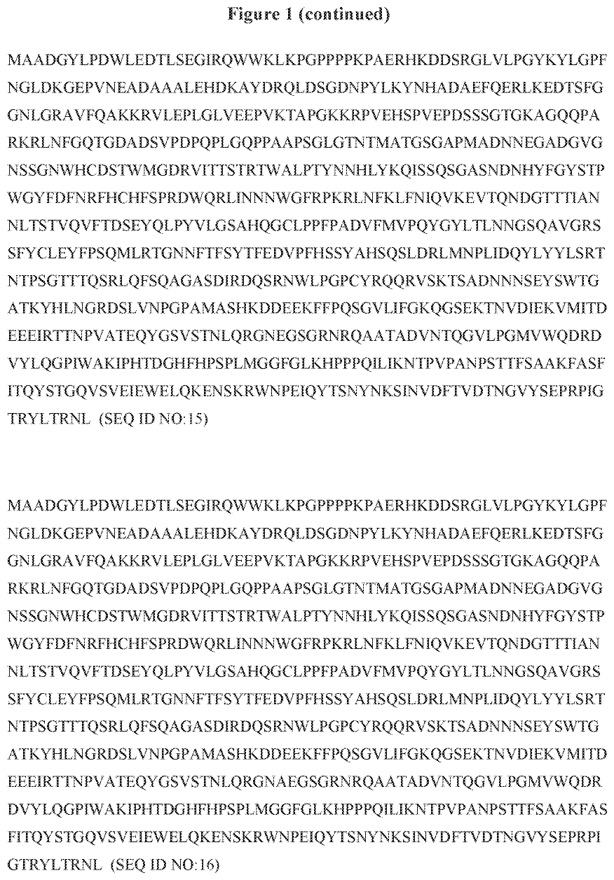

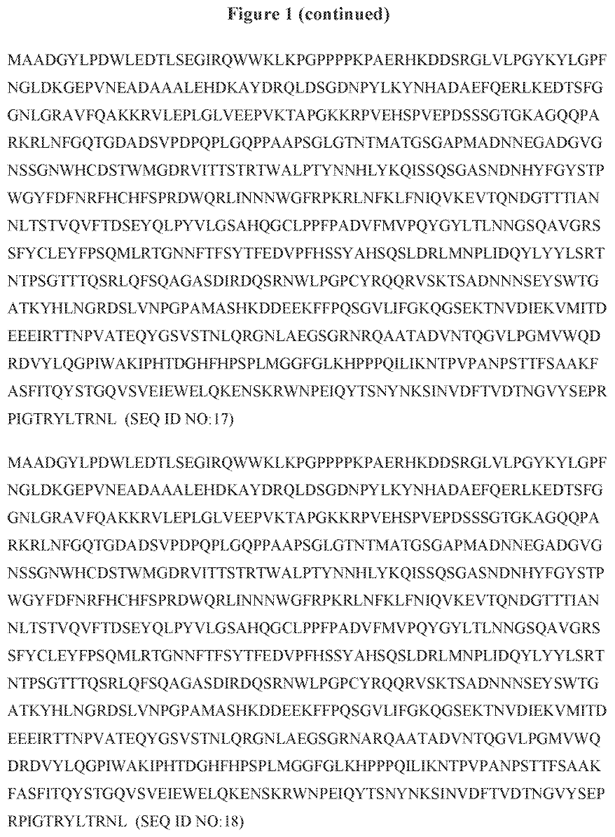

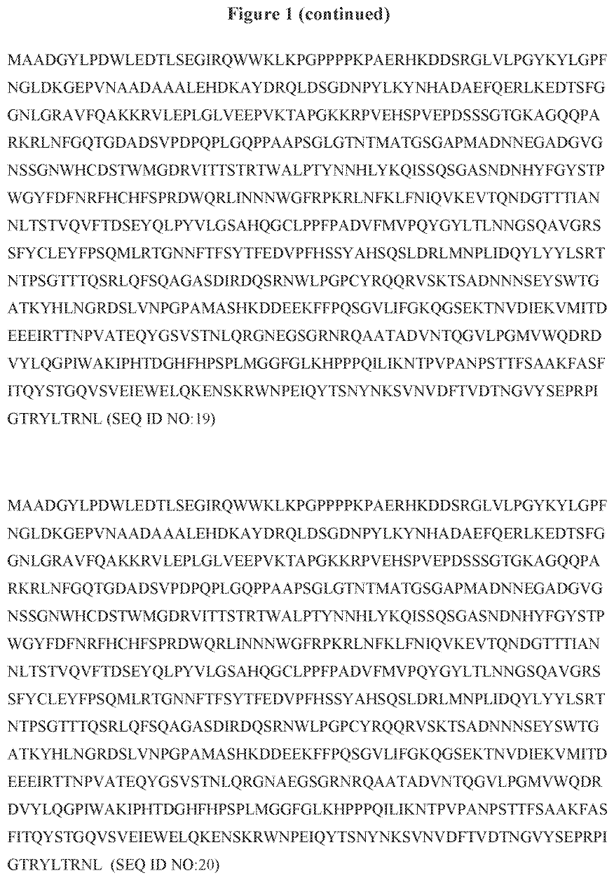

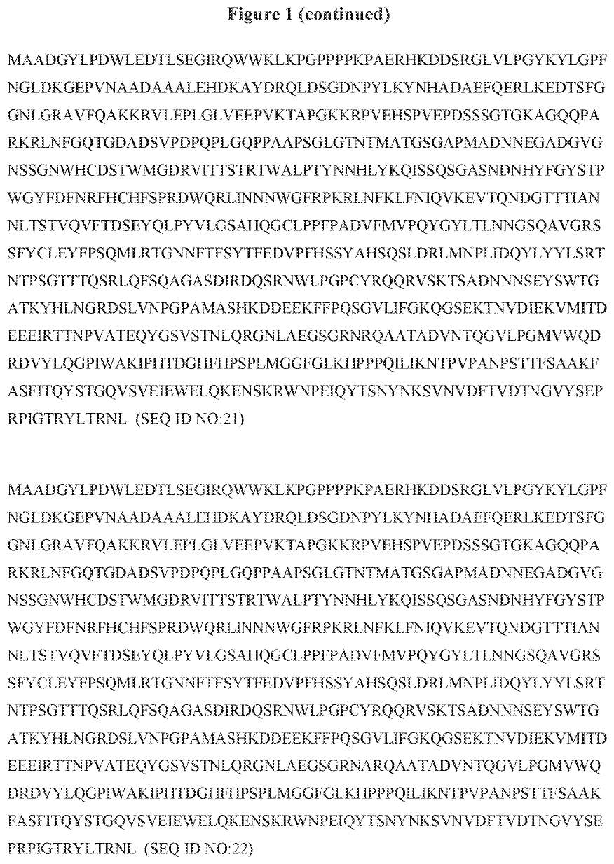

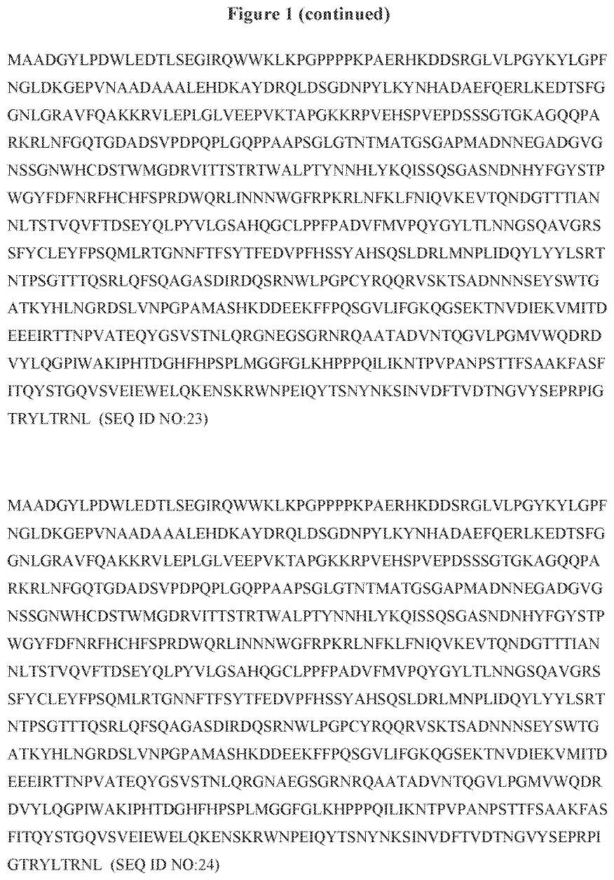

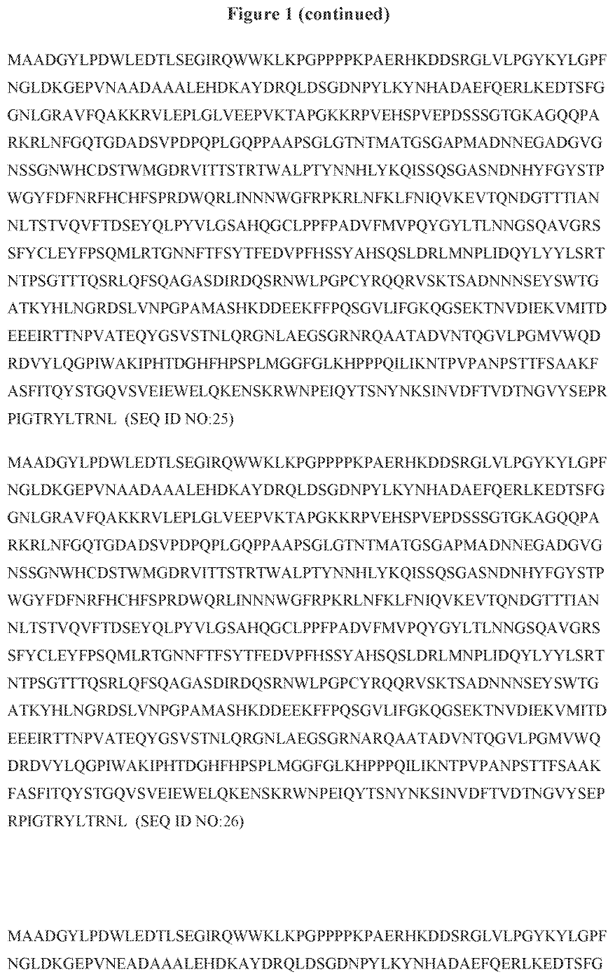

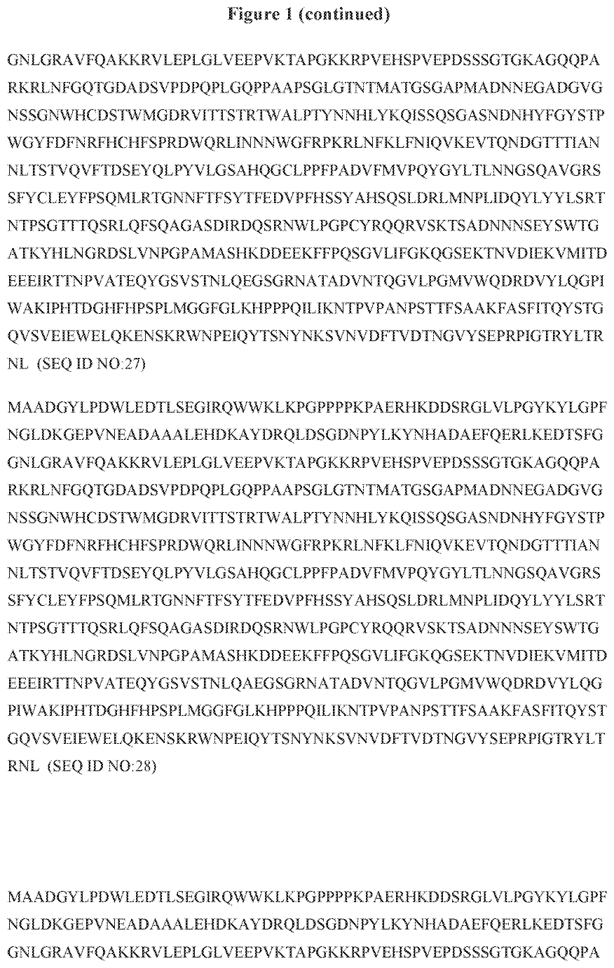

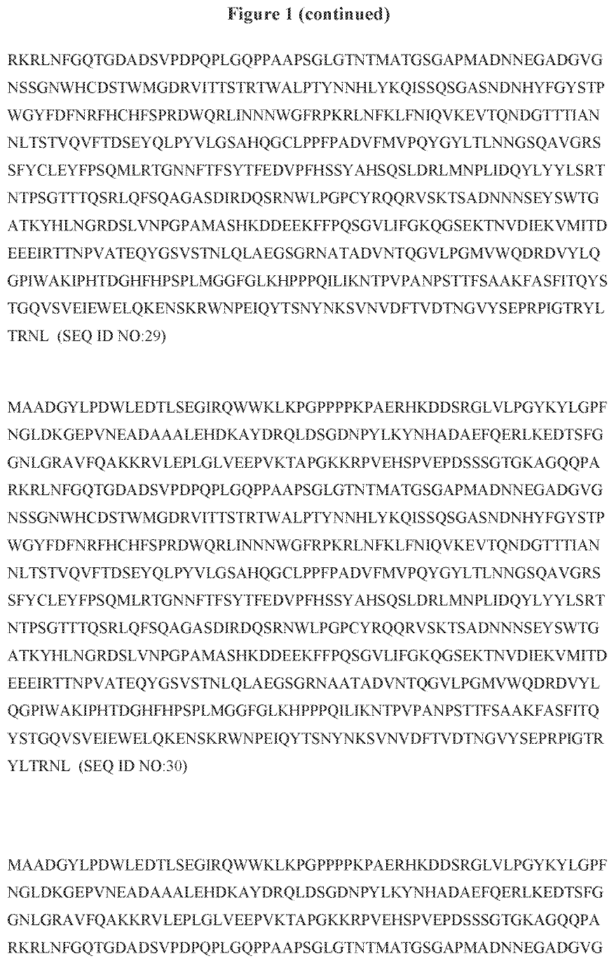

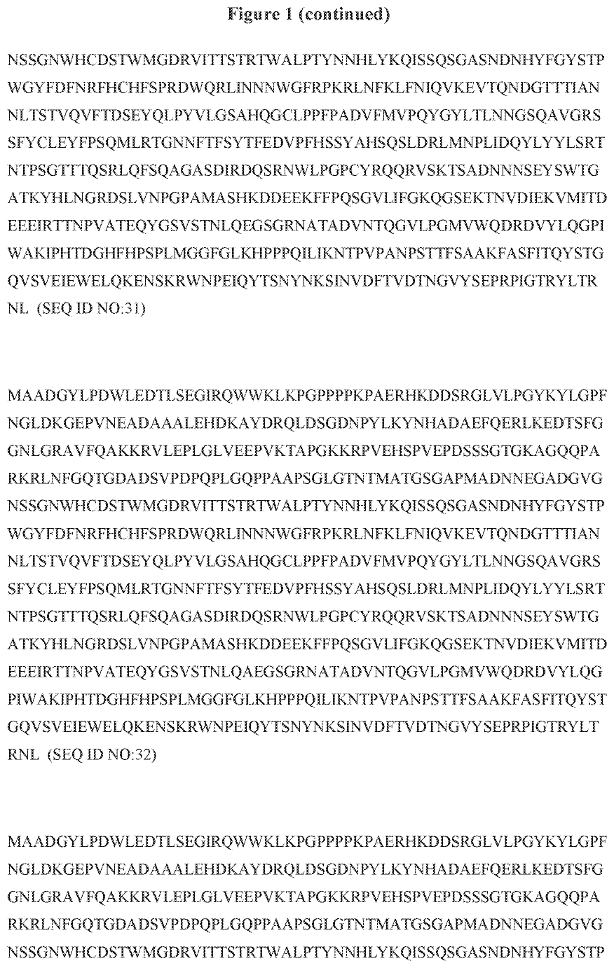

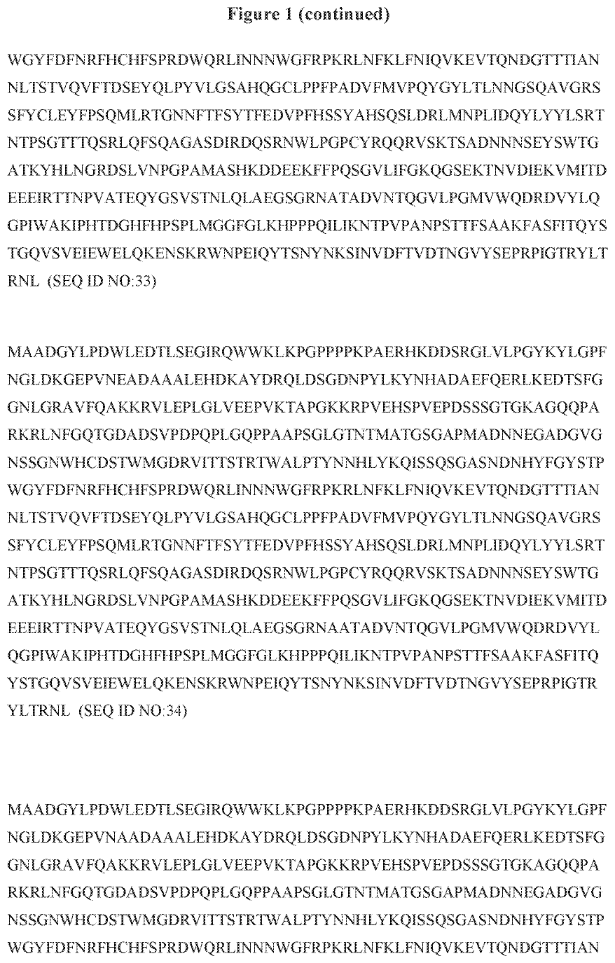

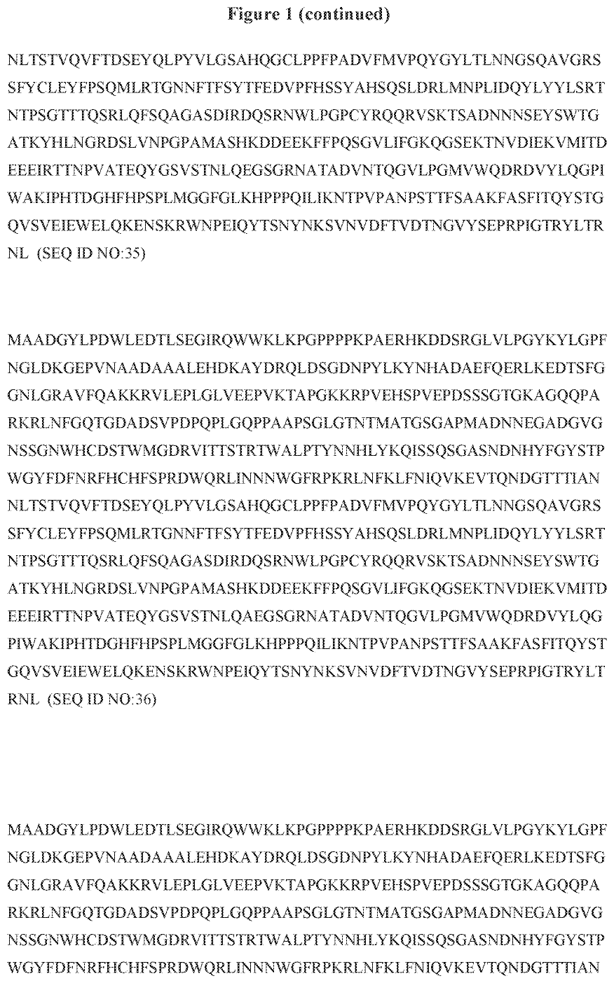

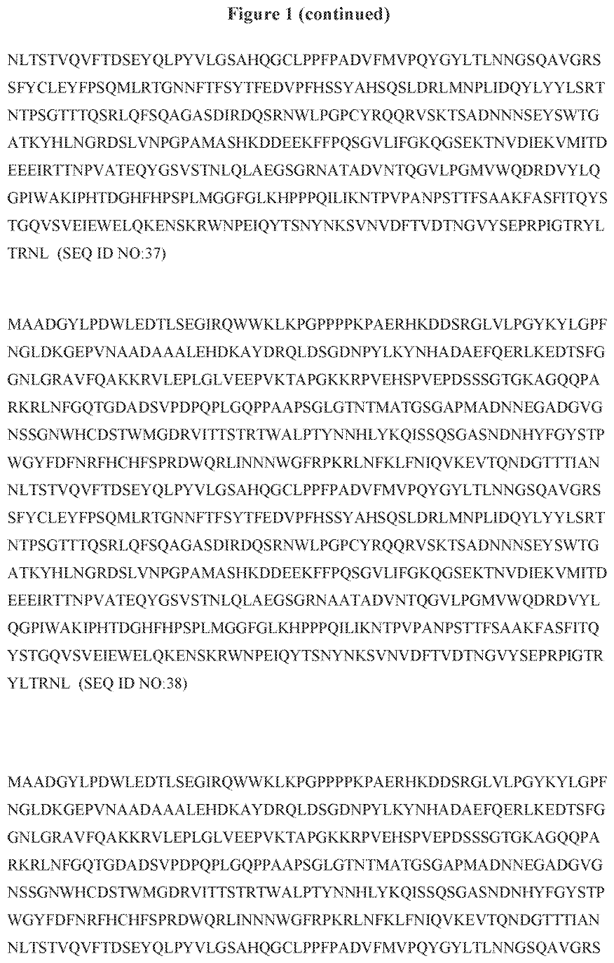

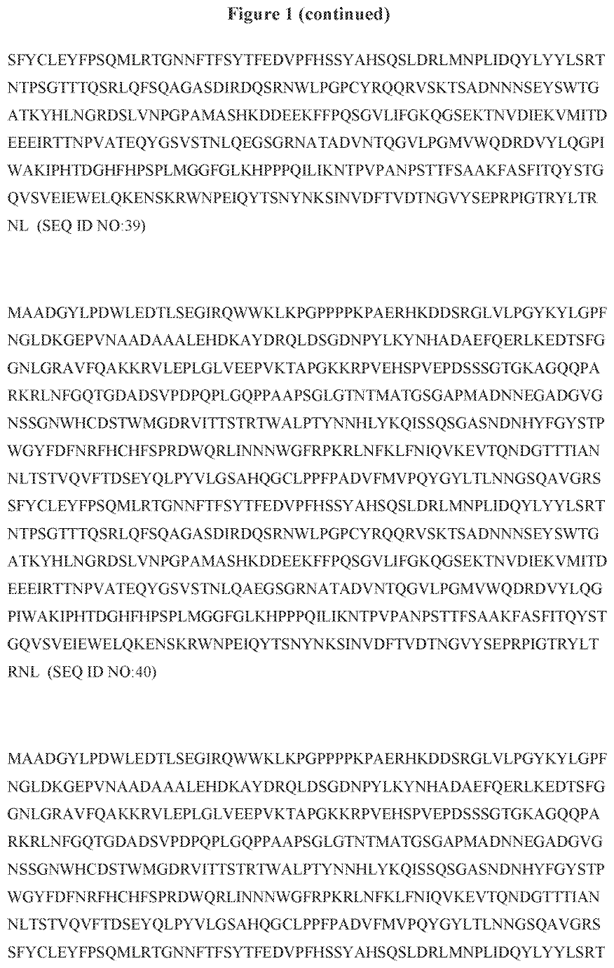

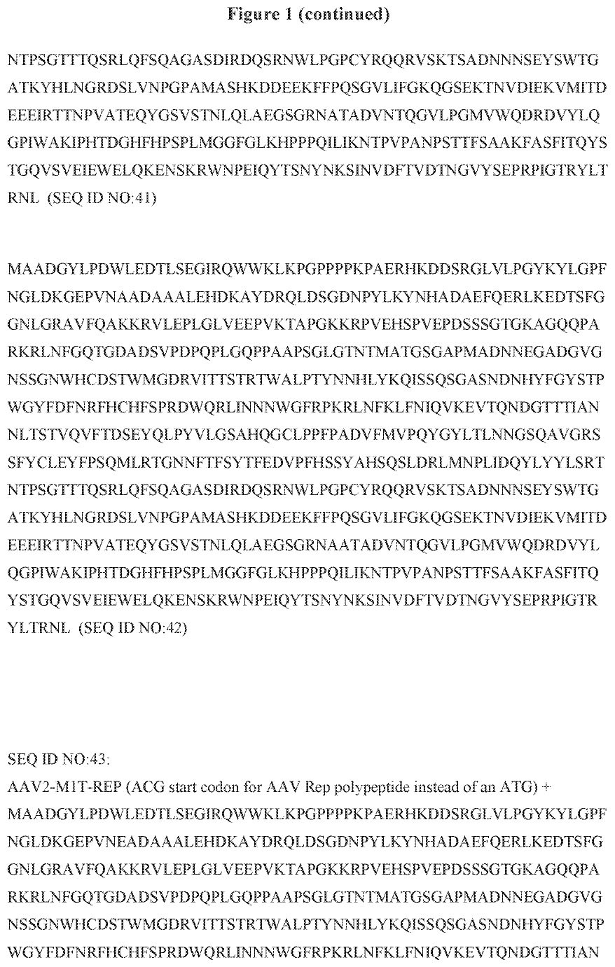

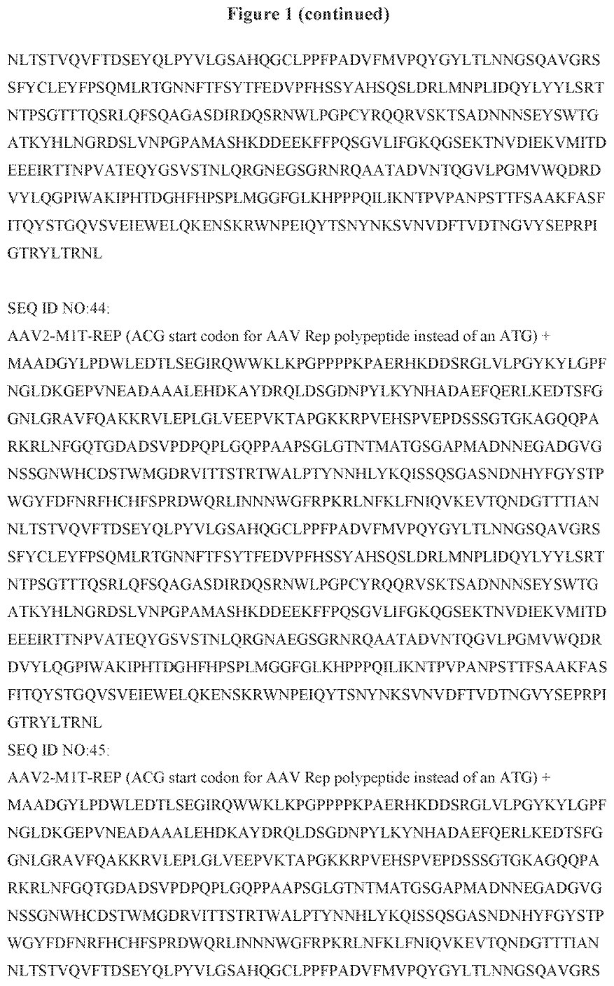

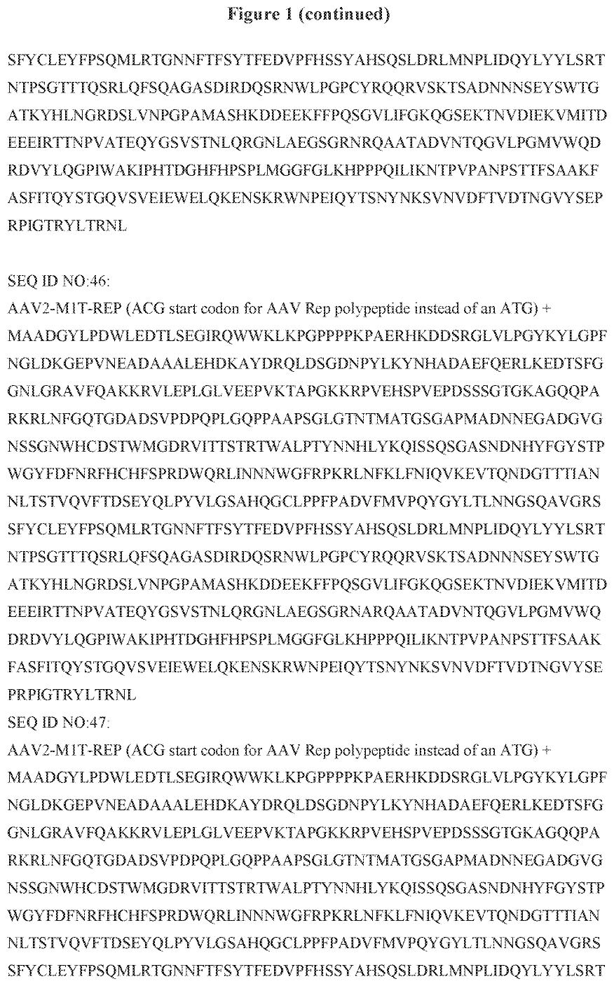

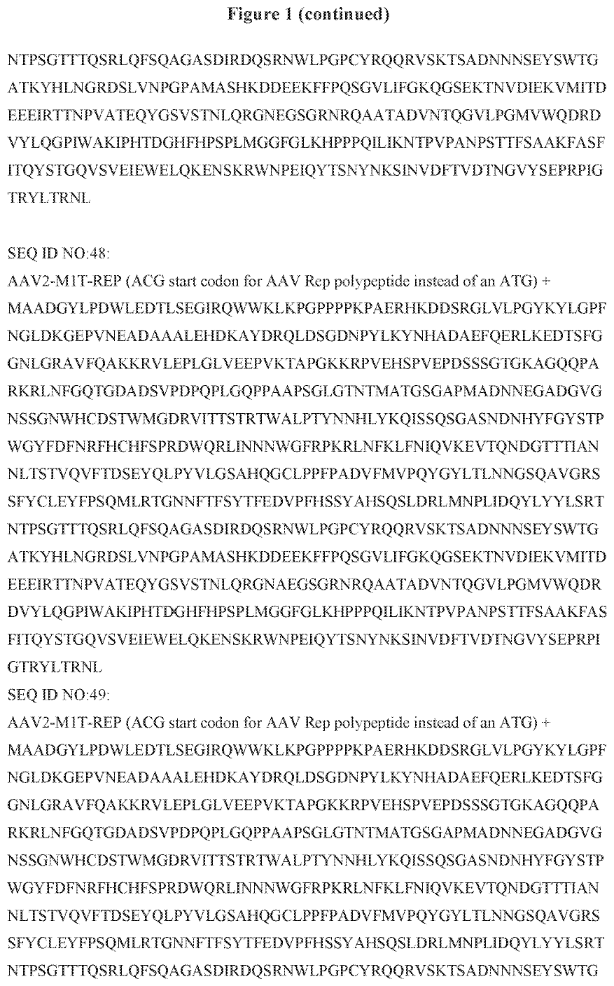

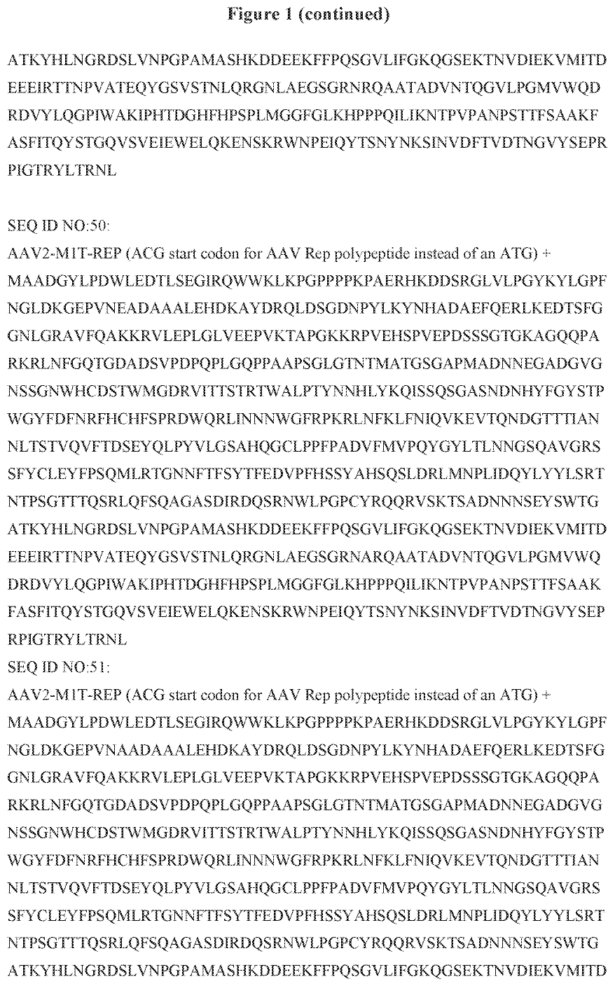

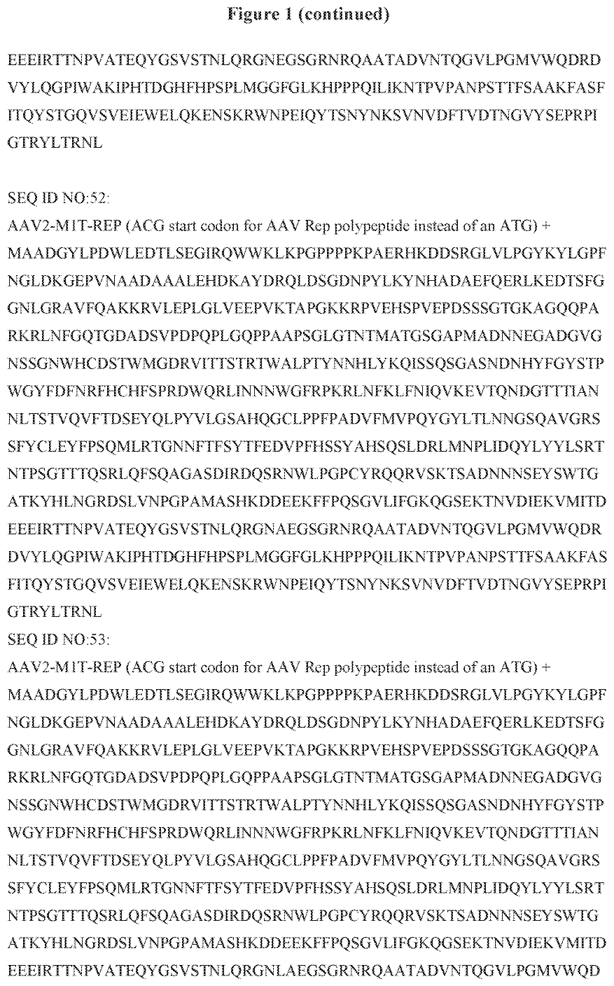

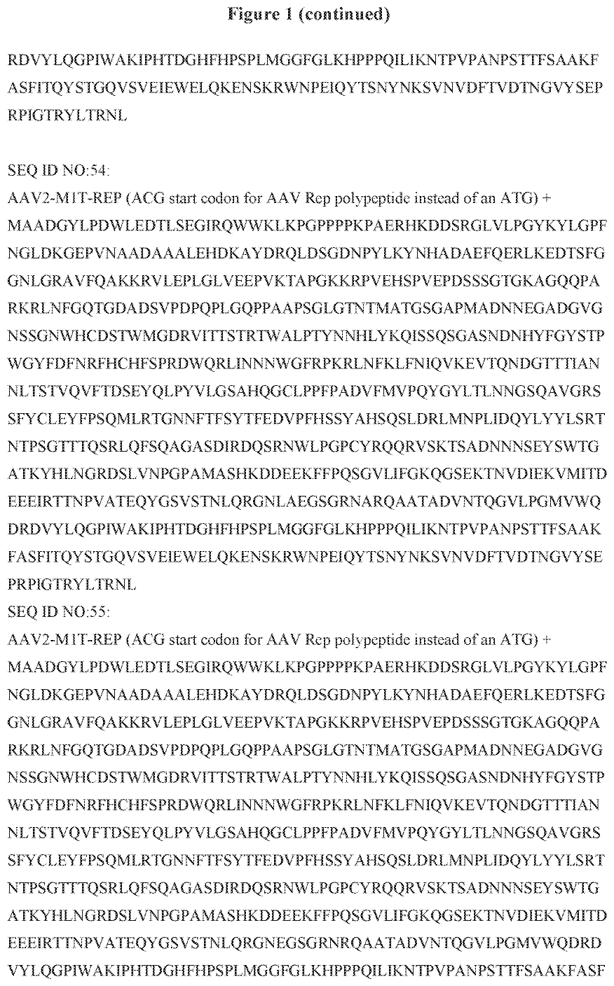

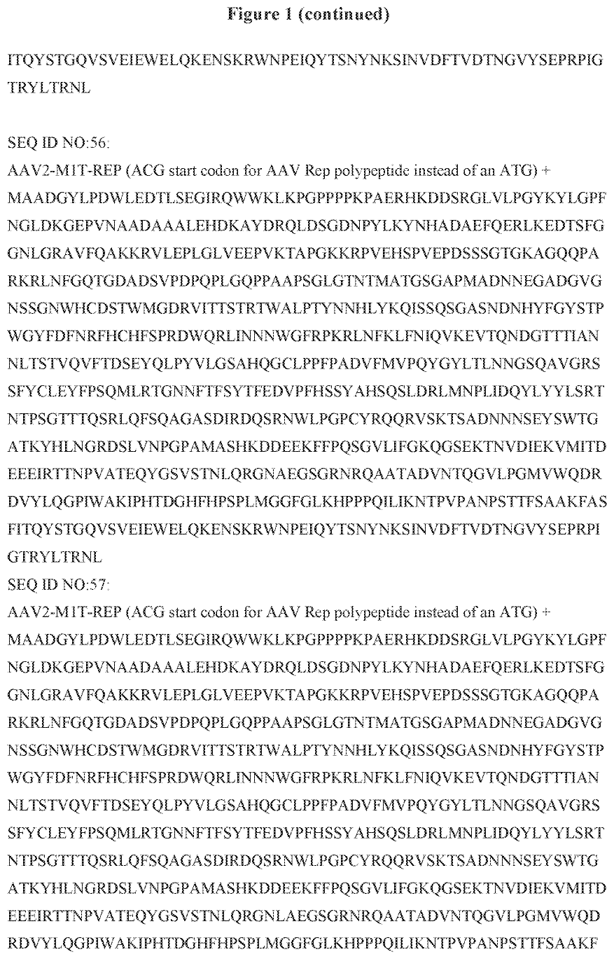

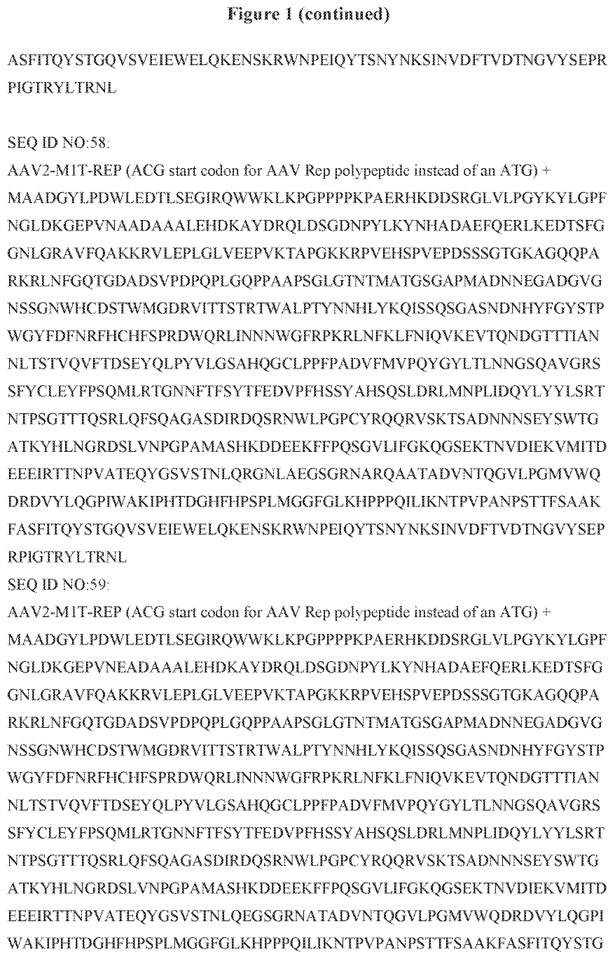

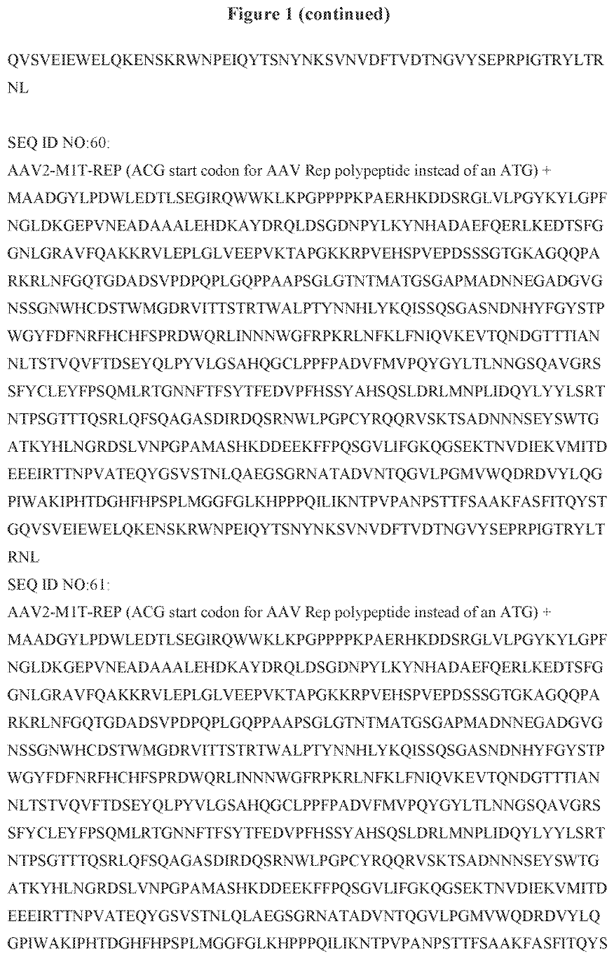

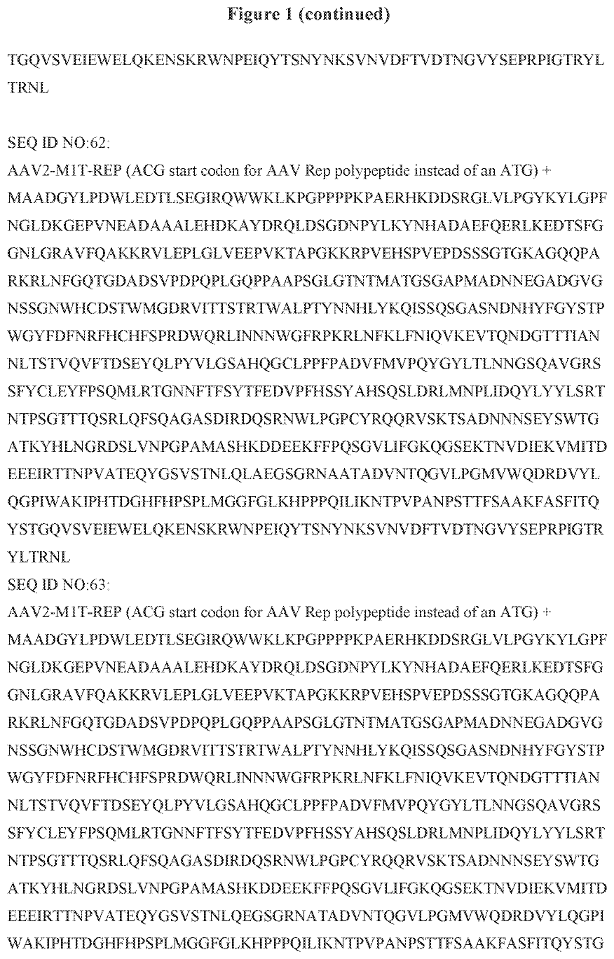

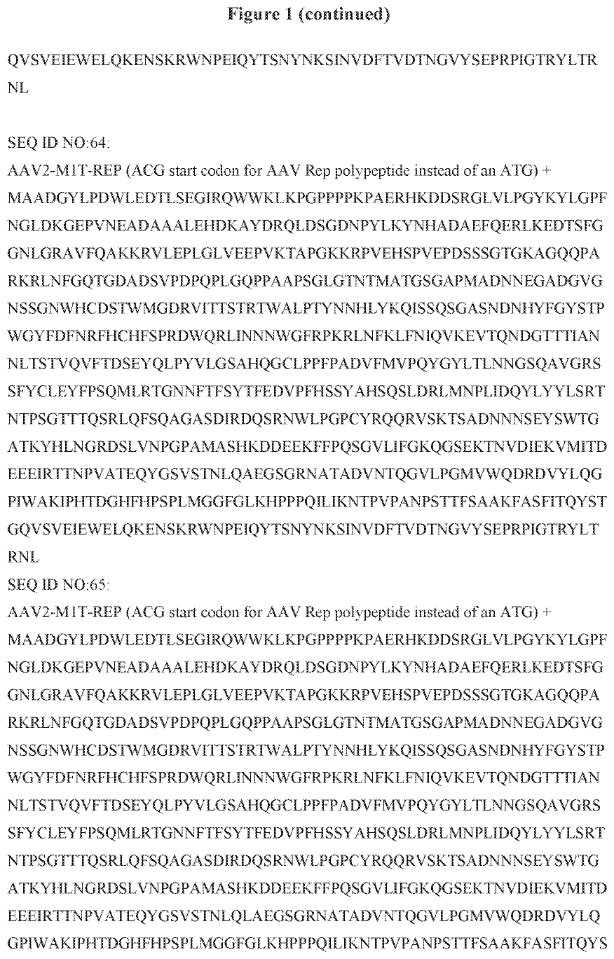

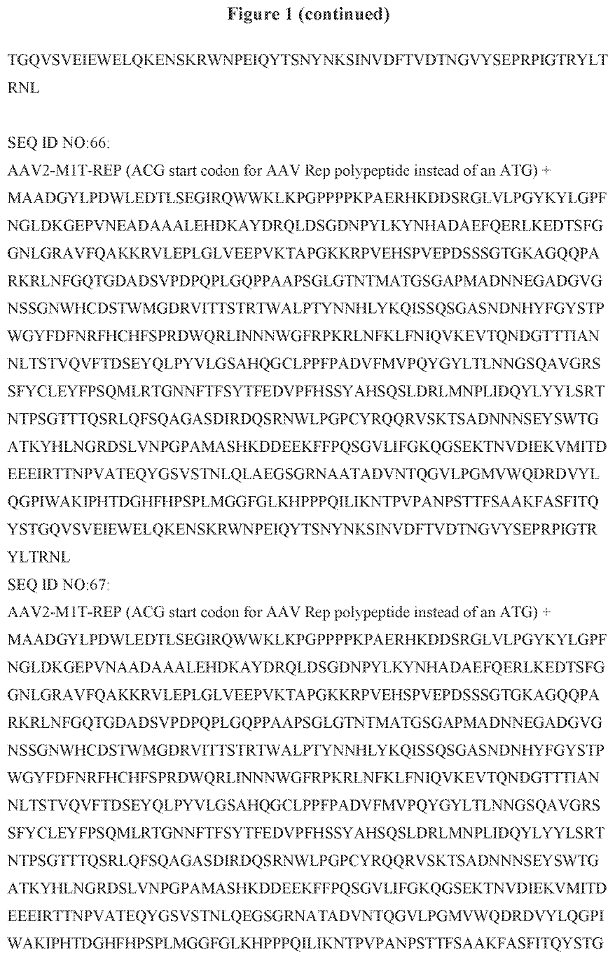

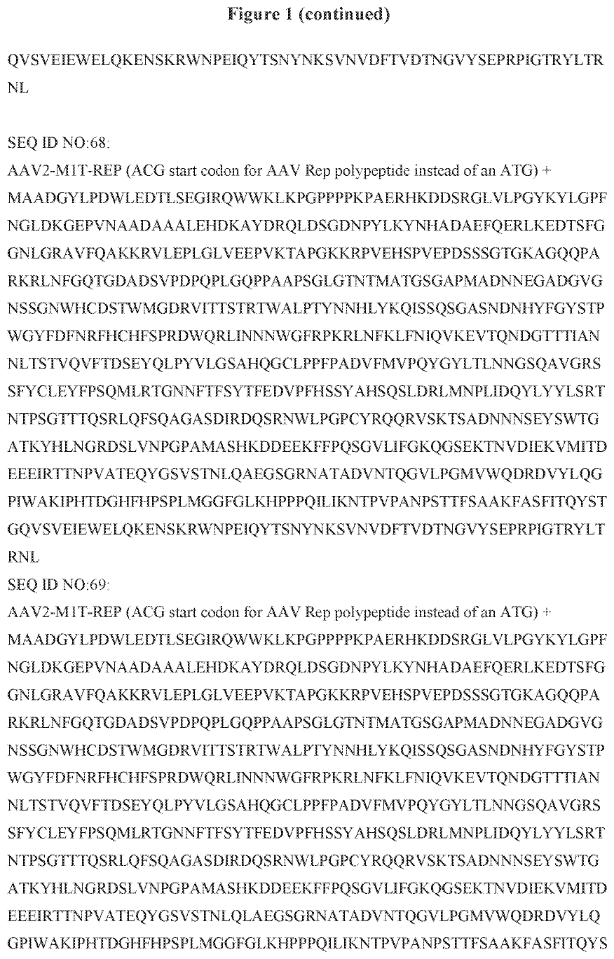

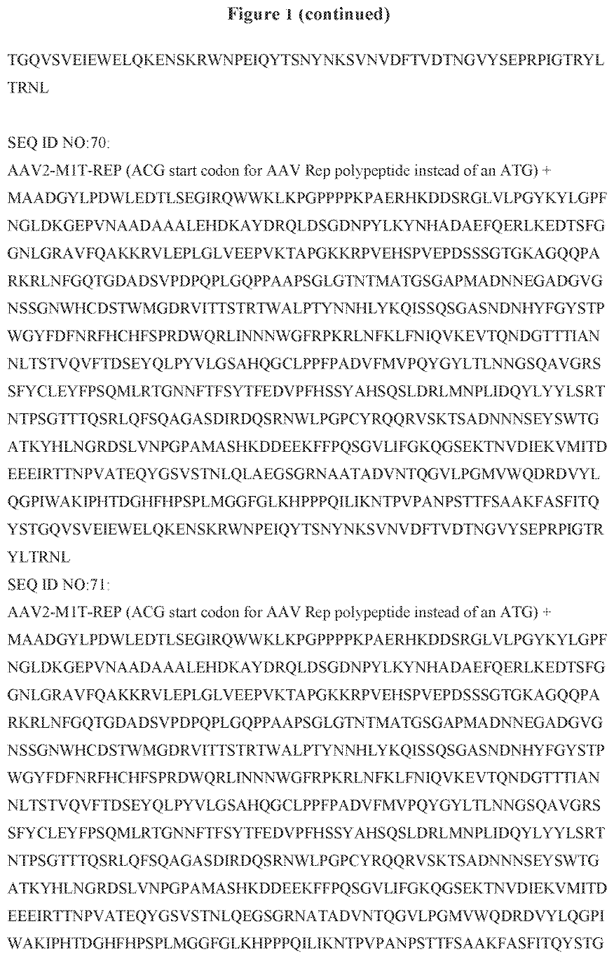

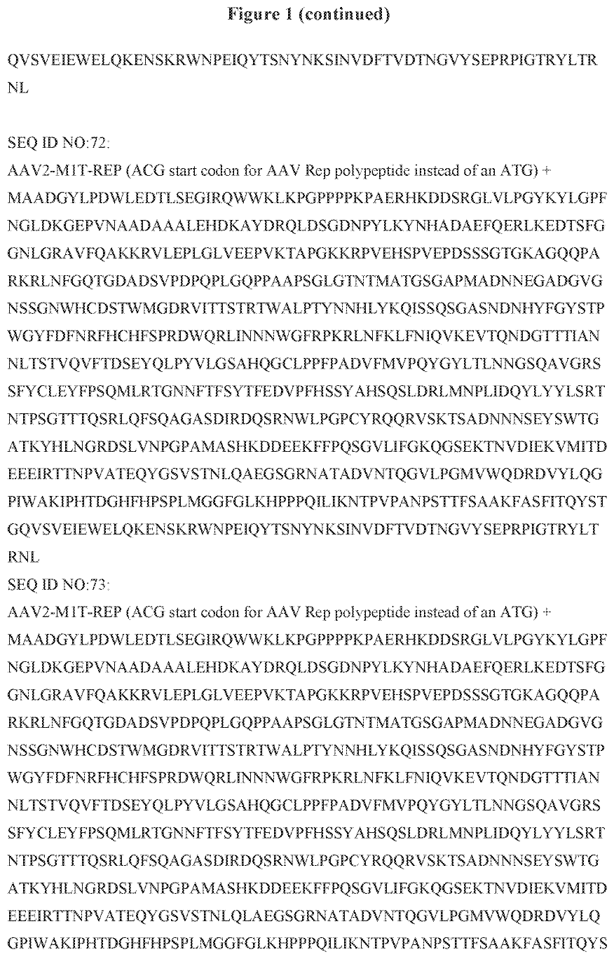

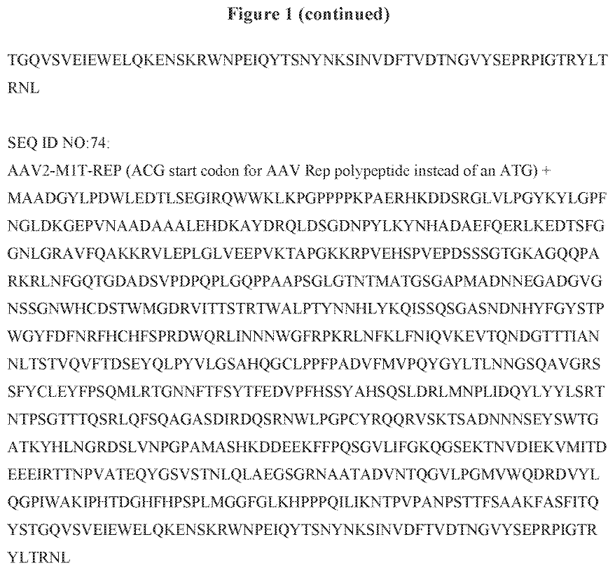

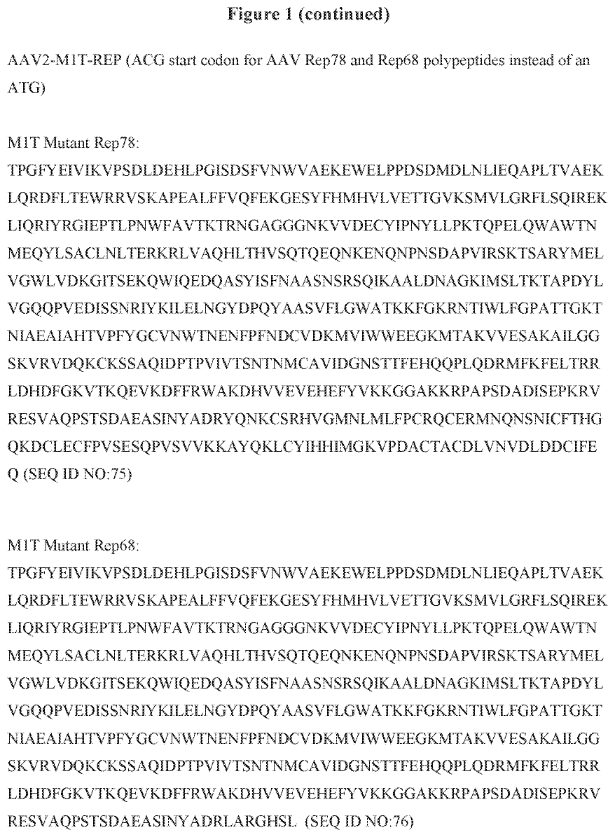

AAV vectors having capsid polypeptides that included SEQ ID NO: 14 (SEQ ID NO:5 located between amino acid residues 587 and 588 of SEQ ID NO:1; see, e.g., FIG. 1) exhibited the ability to infect and drive mRNA expression within retinal cells in a manner similar to that of 7m8 AAV vector. Thus, the AAV vector having capsid polypeptide that included SEQ ID NO: 14 has the ability to exhibit more infectivity of retinal cells than a wild-type AAV2 vector containing an AAV2 capsid polypeptide that consists of SEQ ID NO: 1.

Taken together, these results demonstrate that AAV vectors that include an AAV capsid polypeptide (e.g., an AAV2 capsid polypeptide) having an amino acid sequence set forth in Table 1 (or Formula A) can have the ability to mediate transgene expression in retinal cells.

### Example 2—Treating a Retinal Condition Using an AAV Vector

An AAV vector is constructed to include an AAV2 capsid polypeptide having an amino acid sequence set forth in Table 1 (e.g., SEQ ID NO:2 or 5) (or Formula A) and an exogenous nucleic acid sequence encoding a therapeutic polypeptide. The constructed AAV vector is administered intravitreally to a human identified as having a retinal condition in an amount that is from about 1×10⁷ to about 1×10¹⁴ AAV vectors. After the administration, the severity of one or more symptoms of the retinal condition is reduced and/or the progression of the retinal condition is slowed.

### Example 3—Construction of AAV Vectors Containing Mutated Capsid Polypeptides

A high-throughput method was used to create AAV vectors with mutated capsid polypeptides and to screen those created AAV vectors for particular AAV vectors having the ability to exhibit high efficiency and/or specificity for infecting foveal cones in the retina. See, e.g., Öztürk et al., bioRxiv, 2020.10.01.323196 (2020) and Öztürk et al., eLife, 10: e64175 (2021). Briefly, highly complex libraries of AAV mutants were created and injected into the eyes of primates (cynolmolgus macaques or rhesus macaques). These libraries were created such that each AAV vector in the library contained a unique DNA barcode, which allowed for tracking of a mutated AAV capsid polypeptide. In one library version, successfully packaged AAV vectors were polymerase chain reaction (PCR) amplified and repackaged, resulting in a “repack” library. In another library version, AAV vectors were injected into primate retinas, and nucleic acid encoding the AAV capsid polypeptides were then amplified from the nuclei of foveal cells, resulting in an “enriched” library. Each iteration of the AAV library (e.g., the original library, the repack library, and the enriched library) was injected intravitreally into primate eyes.

After injection, the AAV vectors competed with each other in vivo. Infection of successful AAV vectors led to expression of the DNA barcodes. Single cell suspensions were created from isolated retinal tissue, and single cell microfluidic technology (10× Genomics) was used to create cDNA libraries of individual cells. Computational analysis and/or other desirable characteristics, based on the presence and quantity of DNA barcodes in transcriptomes from thousands of different cells of multiple cell types in parallel. The performance of AAV capsid polypeptides was evaluated on the basis of mRNA transcription levels rather than the presence of DNA, reflecting the ability of the AAV vectors to drive expression of the AAV vector nucleic acid as opposed to simply having the ability to enter a cell.

AAV vectors having capsid polypeptides that included an amino acid sequence insert located between amino acid residues 587 and 588 of SEQ ID NO:1 (18 vectors) or an amino acid sequence insert as a replacement of amino acid residues 585 to 590 of SEQ ID NO: 1 (1 vector) mediated expression in foveal cells. The AAV vectors were ranked based on overall rankings with +++ indicating those that performed in the top ⅓ of vectors tested, with ++ indicating those that performed in middle ⅓ of vectors tested, and with + indicating those that performed in the bottom ⅓ of vectors tested. These were determined in terms of total levels of gene expression in foveal cells. SEQ ID NO: 14 (SEQ ID NO:5 inserted between amino acid residues 587 and 588 of SEQ ID NO: 1; see, e.g., FIG. 1) resulted in “+.” No expression was detected within the limits of detection when the wild-type AAV2 vector was used.

Taken together, these results demonstrate that AAV vectors that include an AAV capsid polypeptide (e.g., an AAV2 capsid polypeptide) having an amino acid sequence set forth in Table 1 (or Formula A) can have the ability to mediate transgene expression in foveal cones following intravitreal injection.

### Example 4—Treating a Retinal Condition Using an AAV Vector

An AAV vector is constructed to include an AAV2 capsid polypeptide having an amino acid sequence set forth in Table 1 (e.g., SEQ ID NO:2 or 5) (or Formula A) and an exogenous nucleic acid sequence encoding a therapeutic polypeptide. The constructed AAV vector is administered intravitreally to a human identified as having a retinal condition in an amount that is from about 1×10⁷ to about 1×10¹⁴ AAV vectors. After the administration, the severity of one or more symptoms of the retinal condition is reduced and/or the progression of the retinal condition is slowed.

### Example 5—Construction of AAV Vectors Containing Mutated Capsid Polypeptides

A high-throughput method was used to create AAV vectors with mutated capsid polypeptides and to screen those created AAV vectors for particular AAV vectors having the ability to exhibit high efficiency and/or specificity for infecting retinal cells. See, e.g., Öztürk et al., bioRxiv, 2020.10.01.323196 (2020) and Öztürk et al., eLife, 10: e64175 (2021). Briefly, highly complex libraries of AAV mutants were created and injected into the eyes of primates (cynolmolgus macaques or rhesus macaques). These libraries were created such that each AAV vector in the library contained a unique DNA barcode, which allowed for tracking of a mutated AAV capsid polypeptide. In one library version, successfully packaged AAV vectors were polymerase chain reaction (PCR) amplified and repackaged, resulting in a “repack” library. In another library version, AAV vectors were injected into primate retinas, and nucleic acid encoding the AAV capsid polypeptides were then amplified from the nuclei of foveal cells, resulting in an “enriched” library. Each iteration of the AAV library (e.g., the original library, the repack library, and the enriched library) was injected intravitreally into primate eyes.

After injection, the AAV vectors competed with each other in vivo. Infection of successful AAV vectors led to expression of the DNA barcodes. Single cell suspensions were created from isolated retinal tissue, and single cell microfluidic technology (10× Genomics) was used to create cDNA libraries of individual cells. Computational analysis was performed to identify optimal vectors, according to cell specificities, expression levels, and/or other desirable characteristics, based on the presence and quantity of DNA barcodes in transcriptomes from thousands of different cells of multiple cell types in parallel. The performance of AAV capsid polypeptides was evaluated on the basis of mRNA transcription levels rather than the presence of DNA, reflecting the ability of the AAV vectors to drive expression of the AAV vector nucleic acid as opposed to simply having the ability to enter a cell.

AAV vectors having capsid polypeptides that included an amino acid sequence insert located between amino acid residues 587 and 588 of SEQ ID NO: 1 (305 total vectors with less than three unique vectors of the total being present within the total more than once) or an amino acid sequence insert as a replacement of amino acid residues 585 to 590 of SEQ ID NO: 1 (19 vectors) mediated expression in retinal cells. The AAV vectors were ranked based on overall rankings with +++ indicating those that performed in the top ⅓ of vectors tested, with ++ indicating those that performed in middle ⅓ of vectors tested, and with + indicating those that performed in the bottom ⅓ of vectors tested. These were determined in terms of total levels of gene expression in retinal cells. SEQ ID NO:14 (SEQ ID NO:5 inserted between amino acid residues 587 and 588 of SEQ ID NO: 1; see, e.g., FIG. 1) resulted in “+++” once, “++” four times, and “+” once. No expression was detected within the limits of detection when the wild-type AAV2 vector was used.

Taken together, these results demonstrate that AAV vectors that include an AAV capsid polypeptide (e.g., an AAV2 capsid polypeptide) having an amino acid sequence set forth in Table 1 (or Formula A) can have the ability to mediate transgene expression (e.g., high expression) in retinal cells following intravitreal injection.

### Example 6—Treating a Retinal Condition Using an AAV Vector

An AAV vector is constructed to include an AAV2 capsid polypeptide having an amino acid sequence set forth in Table I (e.g., SEQ ID NO:2 or 5) (or Formula A) and an exogenous nucleic acid sequence encoding a therapeutic polypeptide. The constructed AAV vector is administered intravitreally to a human identified as having a retinal condition in an amount that is from about 1×10⁷ to about 1×10¹⁴ AAV vectors. After the administration, the severity of one or more symptoms of the retinal condition is reduced and/or the progression of the retinal condition is slowed.

### Example 7—Construction of AAV Vectors Containing Mutated Capsid Polypeptides

A high-throughput method was used to create AAV vectors with mutated capsid polypeptides and to screen those created AAV vectors for particular AAV vectors having the ability to exhibit high efficiency and/or specificity for infecting retinal cells. See, e.g., Öztürk et al., bioRxiv, 2020.10.01.323196 (2020) and Öztürk et al., eLife, 10: e64175 (2021). Briefly, highly complex libraries of AAV mutants were created and injected into the eyes of primates (cynolmolgus macaques or rhesus macaques). These libraries were created such that each AAV vector in the library contained a unique DNA barcode, which allowed for tracking of a mutated AAV capsid polypeptide. In one library version, successfully packaged AAV vectors were polymerase chain reaction (PCR) amplified and repackaged, resulting in a “repack” library. In another library version, AAV vectors were injected into primate retinas, and nucleic acid encoding the AAV capsid polypeptides were then amplified from the nuclei of foveal cells, resulting in an “enriched” library. Each iteration of the AAV library (e.g., the original library, the repack library, and the enriched library) was injected intravitreally into primate eyes.

After injection, the AAV vectors competed with each other in vivo. Infection of successful AAV vectors led to expression of the DNA barcodes. Single cell suspensions were created from isolated retinal tissue, and single cell microfluidic technology (10× Genomics) was used to create cDNA libraries of individual cells. Computational analysis and/or other desirable characteristics, based on the presence and quantity of DNA barcodes in transcriptomes from thousands of different cells of multiple cell types in parallel. The performance of AAV capsid polypeptides was evaluated on the basis of mRNA transcription levels rather than the presence of DNA, reflecting the ability of the AAV vectors to drive expression of the AAV vector nucleic acid as opposed to simply having the ability to enter a cell.

AAV vectors having capsid polypeptides that included an amino acid sequence insert located between amino acid residues 587 and 588 of SEQ ID NO:1 (44 vectors with some duplicates) or an amino acid sequence insert as a replacement of amino acid residues 585 to 590 of SEQ ID NO: 1 (two vectors) mediated expression in retinal cells across two or more retinal regions. The AAV vectors were ranked based on overall rankings with +++ indicating those that performed in the top ⅓ of vectors tested, with ++ indicating those that performed in middle ⅓ of vectors tested, and with + indicating those that performed in the bottom ⅓ of vectors tested. These were determined in terms of total levels of gene expression across retinal regions. SEQ ID NO:14 (SEQ ID NO:5 inserted between amino acid residues 587 and 588 of SEQ ID NO: 1; see, e.g., FIG. 1) resulted in “++” twice. No expression was detected within the limits of detection when the wild-type AAV2 vector was used.

Taken together, these results demonstrate that AAV vectors that include an AAV capsid polypeptide (e.g., an AAV2 capsid polypeptide) having an amino acid sequence set forth in Table 1 (or Formula A) can have the ability to deliver nucleic acid to and express nucleic acid in retinal cells in at least two different retinal regions.

### Example 8—Treating a Retinal Condition Using an AAV Vector

An AAV vector is constructed to include an AAV2 capsid polypeptide having an amino acid sequence set forth in Table 1 (e.g., SEQ ID NO:2 or 5) (or Formula A) and an exogenous nucleic acid sequence encoding a therapeutic polypeptide. The constructed AAV vector is administered intravitreally to a human identified as having a retinal condition in an amount that is from about 1×10⁷ to about 1×10¹⁴ AAV vectors. After the administration, the severity of one or more symptoms of the retinal condition is reduced and/or the progression of the retinal condition is slowed.

### Example 9—Construction of AAV Vectors Containing Mutated Capsid Polypeptides

A high-throughput method was used to create AAV vectors with mutated capsid polypeptides and to screen those created AAV vectors for particular AAV vectors having the ability to exhibit high efficiency and/or specificity for infecting retinal cells of the parafovea region of the eye. See, e.g., Öztürk et al., bioRxiv, 2020.10.01.323196 (2020). Briefly, highly complex libraries of AAV mutants were created and injected into the eyes of primates (cynolmolgus macaques or rhesus macaques). These libraries were created such that each AAV vector in the library contained a unique DNA barcode, which allowed for tracking of a mutated AAV capsid polypeptide. In one library version, successfully packaged AAV vectors were polymerase chain reaction (PCR) amplified and repackaged, resulting in a “repack” library. In another library version, AAV vectors were injected into primate retinas, and nucleic acid encoding the AAV capsid polypeptides were then amplified from the nuclei of foveal cells, resulting in an “enriched” library. Each iteration of the AAV library (e.g., the original library, the repack library, and the enriched library) was injected intravitreally into primate eyes.

After injection, the AAV vectors competed with each other in vivo. Infection of successful AAV vectors led to expression of the DNA barcodes. Single cell suspensions were created from isolated retinal tissue, and single cell microfluidic technology (10× Genomics) was used to create cDNA libraries of individual cells. Computational analysis was performed to identify optimal vectors, according to cell specificities, expression levels, and/or other desirable characteristics, based on the presence and quantity of DNA barcodes in transcriptomes from thousands of different cells of multiple cell types in parallel. The performance of AAV capsid polypeptides was evaluated on the basis of mRNA transcription levels rather than the presence of DNA, reflecting the ability of the AAV vectors to drive expression of the AAV vector nucleic acid as opposed to simply having the ability to enter a cell.

AAV vectors having capsid polypeptides that included an amino acid sequence insert located between amino acid residues 587 and 588 of SEQ ID NO:1 (79 total vectors with less than three unique vectors of the total being present within the total more than once) or an amino acid sequence insert as a replacement of amino acid residues 585 to 590 of SEQ ID NO: 1 (two vectors) mediated expression in retinal cells of the parafovea region. The AAV vectors were ranked based on overall rankings with +++ indicating those that performed in the top ⅓ of vectors tested, with ++ indicating those that performed in middle ⅓ of vectors tested, and with + indicating those that performed in the bottom ⅓ of vectors tested. These were determined in terms of total levels of gene expression in the parafoveal region of the retina. SEQ ID NO:14 (SEQ ID NO:5 inserted between amino acid residues 587 and 588 of SEQ ID NO: 1; see, e.g., FIG. 1) resulted in “++” once and “+” once. No expression was detected within the limits of detection when the wild-type AAV2 vector was used.

Taken together, these results demonstrate that AAV vectors that include an AAV capsid polypeptide (e.g., an AAV2 capsid polypeptide) having an amino acid sequence set forth in Table I (or Formula A) can have the ability to mediate transgene expression in retinal cells of the parafovea region following intravitreal injection.

### Example 10—Treating a Retinal Condition Using an AAV Vector

An AAV vector is constructed to include an AAV2 capsid polypeptide having an amino acid sequence set forth in Table 1 (e.g., SEQ ID NO:2 or 5) (or Formula A) and an exogenous nucleic acid sequence encoding a therapeutic polypeptide. The constructed AAV vector is administered intravitreally to a human identified as having a retinal condition in an amount that is from about 1×10⁷ to about 1×10¹⁴ AAV vectors. After the administration, the severity of one or more symptoms of the retinal condition is reduced and/or the progression of the retinal condition is slowed.

### Example 11—Construction of AAV Vectors Containing Mutated Capsid Polypeptides

A high-throughput method was used to create AAV vectors with mutated capsid polypeptides and to screen those created AAV vectors for particular AAV vectors having the ability to exhibit high efficiency and/or specificity for infecting retinal cells. See, e.g., Öztürk et al., bioRxiv, 2020.10.01.323196 (2020) and Öztürk et al., eLife, 10: e64175 (2021). Briefly, highly complex libraries of AAV mutants were created and injected into the eyes of primates (cynolmolgus macaques or rhesus macaques). These libraries were created such that each AAV vector in the library contained a unique DNA barcode, which allowed for tracking of a mutated AAV capsid polypeptide. In one library version, successfully packaged AAV vectors were polymerase chain reaction (PCR) amplified and repackaged, resulting in a “repack” library. In another library version, AAV vectors were injected into primate retinas, and nucleic acid encoding the AAV capsid polypeptides were then amplified from the nuclei of foveal cells, resulting in an “enriched” library. Each iteration of the AAV library (e.g., the original library, the repack library, and the enriched library) was injected intravitreally into primate eyes.

After injection, the AAV vectors competed with each other in vivo. Infection of successful AAV vectors led to expression of the DNA barcodes. Single cell suspensions were created from isolated retinal tissue, and single cell microfluidic technology (10×Genomics) was used to create cDNA libraries of individual cells. Computational analysis was performed to identify optimal vectors, according to cell specificities, expression levels, and/or other desirable characteristics, based on the presence and quantity of DNA barcodes in transcriptomes from thousands of different cells of multiple cell types in parallel. The performance of AAV capsid polypeptides was evaluated on the basis of mRNA transcription levels rather than the presence of DNA, reflecting the ability of the AAV vectors to drive expression of the AAV vector nucleic acid as opposed to simply having the ability to enter a cell.

AAV vectors having capsid polypeptides that included an amino acid sequence insert located between amino acid residues 587 and 588 of SEQ ID NO:1 (76 total vectors with less than three unique vectors of the total being present within the total more than once) or an amino acid sequence insert as a replacement of amino acid residues 585 to 590 of SEQ ID NO: 1 (8 vectors) mediated expression the ability to deliver nucleic acid to and express nucleic acid in multiple different retinal cells types within an eye, thereby providing an efficient way to obtain nucleic acid delivery to many different retinal cell types. The AAV vectors were ranked based on overall rankings with +++ indicating those that performed in the top ⅓ of vectors tested, with ++ indicating those that performed in middle ⅓ of vectors tested, and with + indicating those that performed in the bottom ⅓ of vectors tested. These were determined in terms of total levels of gene expression across cell types. SEQ ID NO: 14 (SEQ ID NO:5 inserted between amino acid residues 587 and 588 of SEQ ID NO: 1; see, e.g., FIG. 1) resulted in “+.” No expression was detected within the limits of detection when the wild-type AAV2 vector was used.

Taken together, these results demonstrate that AAV vectors that include an AAV capsid polypeptide (e.g., an AAV2 capsid polypeptide) having an amino acid sequence set forth in Table 1 (or Formula A) can have the ability to mediate transgene expression in multiple different retinal cells types within an eye following intravitreal injection.

### Example 12—Treating a Retinal Condition Using an AAV Vector

An AAV vector is constructed to include an AAV2 capsid polypeptide having an amino acid sequence set forth in Table I (e.g., SEQ ID NO:2 or 5) (or Formula A) and an exogenous nucleic acid sequence encoding a therapeutic polypeptide. The constructed AAV vector is administered intravitreally to a human identified as having a retinal condition in an amount that is from about 1×10⁷ to about 1×10¹⁴ AAV vectors. After the administration, the severity of one or more symptoms of the retinal condition is reduced and/or the progression of the retinal condition is slowed.

### Example 13—Construction of AAV Vectors Containing Mutated Capsid Polypeptides

A high-throughput method was used to create AAV vectors with mutated capsid polypeptides and to screen those created AAV vectors for particular AAV vectors having the ability to exhibit high efficiency and/or specificity for infecting retinal cells. See, e.g., Öztürk et al., bioRxiv, 2020.10.01.323196 (2020) and Öztürk et al., eLife, 10: e64175 (2021). Briefly, highly complex libraries of AAV mutants were created and injected into the eyes of primates (cynolmolgus macaques or rhesus macaques). These libraries were created such that each AAV vector in the library contained a unique DNA barcode, which allowed for tracking of a mutated AAV capsid polypeptide. In one library version, successfully packaged AAV vectors were polymerase chain reaction (PCR) amplified and repackaged, resulting in a “repack” library. In another library version, AAV vectors were injected into primate retinas, and nucleic acid encoding the AAV capsid polypeptides were then amplified from the nuclei of foveal cells, resulting in an “enriched” library. Each iteration of the AAV library (e.g., the original library, the repack library, and the enriched library) was injected intravitreally into primate eyes.

After injection, the AAV vectors competed with each other in vivo. Infection of successful AAV vectors led to expression of the DNA barcodes. Single cell suspensions were created from isolated retinal tissue, and single cell microfluidic technology (10× Genomics) was used to create cDNA libraries of individual cells. Computational analysis was performed to identify optimal vectors, according to cell specificities, expression levels, and/or other desirable characteristics, based on the presence and quantity of DNA barcodes in transcriptomes from thousands of different cells of multiple cell types in parallel. The performance of AAV capsid polypeptides was evaluated on the basis of mRNA transcription levels rather than the presence of DNA, reflecting the ability of the AAV vectors to drive expression of the AAV vector nucleic acid as opposed to simply having the ability to enter a cell.

AAV vectors having capsid polypeptides that included an amino acid sequence insert located between amino acid residues 587 and 588 of SEQ ID NO: 1 (10 vectors) mediated expression in RPE cells. The AAV vectors were ranked based on overall rankings with +++ indicating those that performed in the top ⅓ of vectors tested, with ++ indicating those that performed in middle ⅓ of vectors tested, and with + indicating those that performed in the bottom ⅓ of vectors tested. SEQ ID NO: 14 (SEQ ID NO:5 inserted between amino acid residues 587 and 588 of SEQ ID NO: 1; see, e.g., FIG. 1) resulted in “+.” These were determined in terms of total levels of gene expression in RPE cells. No expression was detected within the limits of detection when the wild-type AAV2 vector was used.

Taken together, these results demonstrate that AAV vectors that include an AAV capsid polypeptide (e.g., an AAV2 capsid polypeptide) having an amino acid sequence set forth in Table 1 (or Formula A) can have the ability to mediate transgene expression in RPE cells following intravitreal injection.

### Example 14—Treating a Retinal Condition Using an AAV Vector

An AAV vector is constructed to include an AAV2 capsid polypeptide having an amino acid sequence set forth in Table 1 (e.g., SEQ ID NO:2 or 5) (or Formula A) and an exogenous nucleic acid sequence encoding a therapeutic polypeptide. The constructed AAV vector is administered intravitreally to a human identified as having a retinal condition in an amount that is from about 1×10⁷ to about 1×10¹⁴ AAV vectors. After the administration, the severity of one or more symptoms of the retinal condition is reduced and/or the progression of the retinal condition is slowed.

### Example 15—Construction of AAV Vectors Containing Mutated Capsid Polypeptides

A high-throughput method was used to create AAV vectors with mutated capsid polypeptides and to screen those created AAV vectors for particular AAV vectors having the ability to exhibit high efficiency and/or specificity for infecting retinal cells. See, e.g., Öztürk et al., bioRxiv, 2020.10.01.323196 (2020) and Öztürk et al., eLife, 10:064175 (2021). Briefly, highly complex libraries of AAV mutants were created and injected into the eyes of primates (cynolmolgus macaques or rhesus macaques). These libraries were created such that each AAV vector in the library contained a unique DNA barcode, which allowed for tracking of a mutated AAV capsid polypeptide. In one library version, successfully packaged AAV vectors were polymerase chain reaction (PCR) amplified and repackaged, resulting in a “repack” library. In another library version, AAV vectors were injected into primate retinas, and nucleic acid encoding the AAV capsid polypeptides were then amplified from the nuclei of foveal cells, resulting in an “enriched” library. Each iteration of the AAV library (e.g., the original library, the repack library, and the enriched library) was injected intravitreally into primate eyes.

After injection, the AAV vectors competed with each other in vivo. Infection of successful AAV vectors led to expression of the DNA barcodes. Single cell suspensions were created from isolated retinal tissue, and single cell microfluidic technology (10× Genomics) was used to create cDNA libraries of individual cells. Computational analysis was performed to identify optimal vectors, according to cell specificities, expression levels, and/or other desirable characteristics, based on the presence and quantity of DNA barcodes in transcriptomes from thousands of different cells of multiple cell types in parallel. The performance of AAV capsid polypeptides was evaluated on the basis of mRNA transcription levels rather than the presence of DNA, reflecting the ability of the AAV vectors to drive expression of the AAV vector nucleic acid as opposed to simply having the ability to enter a cell.

AAV vectors having capsid polypeptides that included an amino acid sequence insert located between amino acid residues 587 and 588 of SEQ ID NO:1 (72 total vectors with less than three unique vectors of the total being present within the total more than once) or an amino acid sequence insert as a replacement of amino acid residues 585 to 590 of SEQ ID NO: 1 (7 vectors) mediated expression preferentially in photoreceptor cells. The AAV vectors were ranked based on overall rankings with +++ indicating those that performed in the top ⅓ of vectors tested, with ++ indicating those that performed in middle ⅓ of vectors tested, and with + indicating those that performed in the bottom ⅓ of vectors tested. These were determined in terms of total levels of gene expression in retinal cells. SEQ ID NO:14 (SEQ ID NO:5 inserted between amino acid residues 587 and 588 of SEQ ID NO: 1; see, e.g., FIG. 1) resulted in “+++” once, “++” once, and “+” once. No expression was detected within the limits of detection when the wild-type AAV2 vector was used.

Taken together, these results demonstrate that AAV vectors that include an AAV capsid polypeptide (e.g., an AAV2 capsid polypeptide) having an amino acid sequence set forth in Table 1 (or Formula A) can have the ability to mediate transgene expression preferentially in photoreceptor cells following intravitreal injection.

### Example 16—Treating a Retinal Condition Using an AAV Vector

An AAV vector is constructed to include an AAV2 capsid polypeptide having an amino acid sequence set forth in Table 1 (e.g., SEQ ID NO:2 or 5) (or Formula A) and an exogenous nucleic acid sequence encoding a therapeutic polypeptide. The constructed AAV vector is administered intravitreally to a human identified as having a retinal condition in an amount that is from about 1×10⁷ to about 1×10¹⁴ AAV vectors. After the administration, the severity of one or more symptoms of the retinal condition is reduced and/or the progression of the retinal condition is slowed.

### Example 17—Construction of AAV Vectors Containing Mutated Capsid Polypeptides

A high-throughput method was used to create AAV vectors with mutated capsid polypeptides and to screen those created AAV vectors for particular AAV vectors having the ability to exhibit high efficiency and/or specificity for infecting retinal cells. See, e.g., Öztürk et al., bioRxiv, 2020.10.01.323196 (2020) and Öztürk et al., eLife, 10: e64175 (2021). Briefly, highly complex libraries of AAV mutants were created and injected into the eyes of primates (cynolmolgus macaques or rhesus macaques). These libraries were created such that each AAV vector in the library contained a unique DNA barcode, which allowed for tracking of a mutated AAV capsid polypeptide. In one library version, successfully packaged AAV vectors were polymerase chain reaction (PCR) amplified and repackaged, resulting in a “repack” library. In another library version, AAV vectors were injected into primate retinas, and nucleic acid encoding the AAV capsid polypeptides were then amplified from the nuclei of foveal cells, resulting in an “enriched” library. Each iteration of the AAV library (e.g., the original library, the repack library, and the enriched library) was injected intravitreally into primate eyes.

After injection, the AAV vectors competed with each other in vivo. Infection of successful AAV vectors led to expression of the DNA barcodes. Single cell suspensions were created from isolated retinal tissue, and single cell microfluidic technology (10× Genomics) was used to create cDNA libraries of individual cells. Computational analysis was performed to identify optimal vectors, according to cell specificities, expression levels, and/or other desirable characteristics, based on the presence and quantity of DNA barcodes in transcriptomes from thousands of different cells of multiple cell types in parallel. The performance of AAV capsid polypeptides was evaluated on the basis of mRNA transcription levels rather than the presence of DNA, reflecting the ability of the AAV vectors to drive expression of the AAV vector nucleic acid as opposed to simply having the ability to enter a cell.

AAV vectors having capsid polypeptides that included an amino acid sequence insert located between amino acid residues 587 and 588 of SEQ ID NO: 1 (248 total vectors with less than three unique vectors of the total being present within the total more than once) or an amino acid sequence insert as a replacement of amino acid residues 585 to 590 of SEQ ID NO: 1 (15 vectors) mediated expression preferentially in retinal ganglion cells. The AAV vectors were ranked based on overall rankings with +++ indicating those that performed in the top ⅓ of vectors tested, with ++ indicating those that performed in middle ⅓ of vectors tested, and with + indicating those that performed in the bottom ⅓ of vectors tested. These were determined in terms of total levels of gene expression in retinal cells. SEQ ID NO:14 (SEQ ID NO:5 inserted between amino acid residues 587 and 588 of SEQ ID NO: 1; see, e.g., FIG. 1) resulted in “+++” once, “++” twice, and “+” once. No expression was detected within the limits of detection when the wild-type AAV2 vector was used.

Taken together, these results demonstrate that AAV vectors that include an AAV capsid polypeptide (e.g., an AAV2 capsid polypeptide) having an amino acid sequence set forth in Table 1 (or Formula A) can have the ability to mediate transgene expression preferentially in retinal ganglion cells following intravitreal injection.

### Example 18—Treating a Retinal Condition Using an AAV Vector

An AAV vector is constructed to include an AAV2 capsid polypeptide having an amino acid sequence set forth in Table 1 (e.g., SEQ ID NO:2 or 5) (or Formula A) and an exogenous nucleic acid sequence encoding a therapeutic polypeptide. The constructed AAV vector is administered intravitreally to a human identified as having a retinal condition in an amount that is from about 1×10⁷ to about 1×10¹⁴ AAV vectors. After the administration, the severity of one or more symptoms of the retinal condition is reduced and/or the progression of the retinal condition is slowed.

### Example 19—Construction of AAV Vectors Containing Mutated Capsid Polypeptides

A high-throughput method was used to create AAV vectors with mutated capsid polypeptides and to screen those created AAV vectors for particular AAV vectors having the ability to exhibit high efficiency and/or specificity for infecting retinal cells. See, e.g., Öztürk et al., bioRxiv, 2020.10.01.323196 (2020) and Öztürk et al., eLife, 10: e64175 (2021). Briefly, highly complex libraries of AAV mutants were created and injected into the eyes of primates (cynolmolgus macaques or rhesus macaques). These libraries were created such that each AAV vector in the library contained a unique DNA barcode, which allowed for tracking of a mutated AAV capsid polypeptide. In one library version, successfully packaged AAV vectors were polymerase chain reaction (PCR) amplified and repackaged, resulting in a “repack” library. In another library version, AAV vectors were injected into primate retinas, and nucleic acid encoding the AAV capsid polypeptides were then amplified from the nuclei of foveal cells, resulting in an “enriched” library. Each iteration of the AAV library (e.g., the original library, the repack library, and the enriched library) was injected intravitreally into primate eyes.

After injection, the AAV vectors competed with each other in vivo. Infection of successful AAV vectors led to expression of the DNA barcodes. Single cell suspensions were created from isolated retinal tissue, and single cell microfluidic technology (10× Genomics) was used to create cDNA libraries of individual cells. Computational analysis was performed to identify optimal vectors, according to cell specificities, expression levels, and/or other desirable characteristics, based on the presence and quantity of DNA barcodes in transcriptomes from thousands of different cells of multiple cell types in parallel. The performance of AAV capsid polypeptides was evaluated on the basis of mRNA transcription levels rather than the presence of DNA, reflecting the ability of the AAV vectors to drive expression of the AAV vector nucleic acid as opposed to simply having the ability to enter a cell.

AAV vectors having capsid polypeptides that included an amino acid sequence insert located between amino acid residues 587 and 588 of SEQ ID NO:1 (67 vectors) or an amino acid sequence insert as a replacement of amino acid residues 585 to 590 of SEQ ID NO: 1 (7 vectors) mediated expression preferentially in bipolar cells. The AAV vectors were ranked based on overall rankings with +++ indicating those that performed in the top ⅓ of vectors tested, with ++ indicating those that performed in middle ⅓ of vectors tested, and with + indicating those that performed in the bottom ⅓ of vectors tested. These were determined in terms of total levels of gene expression in retinal cells. SEQ ID NO:14 (SEQ ID NO:5 inserted between amino acid residues 587 and 588 of SEQ ID NO: 1; see, e.g., FIG. 1) resulted in “+.” No expression was detected within the limits of detection when the wild-type AAV2 vector was used.

Taken together, these results demonstrate that AAV vectors that include an AAV capsid polypeptide (e.g., an AAV2 capsid polypeptide) having an amino acid sequence set forth in Table 1 (or Formula A) can have the ability to mediate transgene expression preferentially in bipolar cells following intravitreal injection.

### Example 20—Treating a Retinal Condition Using an AAV Vector

An AAV vector is constructed to include an AAV2 capsid polypeptide having an amino acid sequence set forth in Table 1 (e.g., SEQ ID NO:2 or 5) (or Formula A) and an exogenous nucleic acid sequence encoding a therapeutic polypeptide. The constructed AAV vector is administered intravitreally to a human identified as having a retinal condition in an amount that is from about 1×10⁷ to about 1×10¹⁴ AAV vectors. After the administration, the severity of one or more symptoms of the retinal condition is reduced and/or the progression of the retinal condition is slowed.

### Example 21—Construction of AAV Vectors Containing Mutated Capsid Polypeptides

A high-throughput method was used to create AAV vectors with mutated capsid polypeptides and to screen those created AAV vectors for particular AAV vectors having the ability to exhibit high efficiency and/or specificity for infecting retinal cells. See, e.g., Öztürk et al., bioRxiv, 2020.10.01.323196 (2020) and Öztürk et al., eLife, 10:064175 (2021). Briefly, highly complex libraries of AAV mutants were created and injected into the eyes of primates (cynolmolgus macaques or rhesus macaques). These libraries were created such that each AAV vector in the library contained a unique DNA barcode, which allowed for tracking of a mutated AAV capsid polypeptide. In one library version, successfully packaged AAV vectors were polymerase chain reaction (PCR) amplified and repackaged, resulting in a “repack” library. In another library version, AAV vectors were injected into primate retinas, and nucleic acid encoding the AAV capsid polypeptides were then amplified from the nuclei of foveal cells, resulting in an “enriched” library. Each iteration of the AAV library (e.g., the original library, the repack library, and the enriched library) was injected intravitreally into primate eyes.

After injection, the AAV vectors competed with each other in vivo. Infection of successful AAV vectors led to expression of the DNA barcodes. Single cell suspensions were created from isolated retinal tissue, and single cell microfluidic technology (10× Genomics) was used to create cDNA libraries of individual cells. Computational analysis was performed to identify optimal vectors, according to cell specificities, expression levels, and/or other desirable characteristics, based on the presence and quantity of DNA barcodes in transcriptomes from thousands of different cells of multiple cell types in parallel. The performance of AAV capsid polypeptides was evaluated on the basis of mRNA transcription levels rather than the presence of DNA, reflecting the ability of the AAV vectors to drive expression of the AAV vector nucleic acid as opposed to simply having the ability to enter a cell.

AAV vectors having capsid polypeptides that included an amino acid sequence insert located between amino acid residues 587 and 588 of SEQ ID NO:1 (61 vectors) or an amino acid sequence insert as a replacement of amino acid residues 585 to 590 of SEQ ID NO:1 (3 vectors) mediated expression preferentially in OFF-retinal ganglion cells. The AAV vectors were ranked based on overall rankings with +++ indicating those that performed in the top ⅓ of vectors tested, with ++ indicating those that performed in middle ⅓ of vectors tested, and with + indicating those that performed in the bottom ⅓ of vectors tested. These were determined in terms of total levels of gene expression in retinal cells. SEQ ID NO:14 (SEQ ID NO: 5 inserted between amino acid residues 587 and 588 of SEQ ID NO: 1; see, e.g., FIG. 1) resulted in “+++.” No expression was detected within the limits of detection when the wild-type AAV2 vector was used.

Taken together, these results demonstrate that AAV vectors that include an AAV capsid polypeptide (e.g., an AAV2 capsid polypeptide) having an amino acid sequence set forth in Table 1 (or Formula A) can have the ability to mediate transgene expression preferentially in OFF-retinal ganglion cells following intravitreal injection.

### Example 22—Treating a Retinal Condition Using an AAV Vector

An AAV vector is constructed to include an AAV2 capsid polypeptide having an amino acid sequence set forth in Table 1 (e.g., SEQ ID NO:2 or 5) (or Formula A) and an exogenous nucleic acid sequence encoding a therapeutic polypeptide. The constructed AAV vector is administered intravitreally to a human identified as having a retinal condition in an amount that is from about 1×10⁷ to about 1×10¹⁴ AAV vectors. After the administration, the severity of one or more symptoms of the retinal condition is reduced and/or the progression of the retinal condition is slowed.

### Example 23—Construction of AAV Vectors Containing Mutated Capsid Polypeptides

A high-throughput method was used to create AAV vectors with mutated capsid polypeptides and to screen those created AAV vectors for particular AAV vectors having the ability to exhibit high efficiency and/or specificity for infecting retinal cells. See, e.g., Öztürk et al., bioRxiv, 2020.10.01.323196 (2020) and Öztürk et al., eLife, 10: e64175 (2021). Briefly, highly complex libraries of AAV mutants were created and injected into the eyes of primates (cynolmolgus macaques or rhesus macaques). These libraries were created such that each AAV vector in the library contained a unique DNA barcode, which allowed for tracking of a mutated AAV capsid polypeptide. In one library version, successfully packaged AAV vectors were polymerase chain reaction (PCR) amplified and repackaged, resulting in a “repack” library.

The performance of AAV capsid polypeptides for packaging was evaluated on the basis of successful packaging in the original and the “repack” library, reflecting the ability of the AAV vectors to package.

AAV vectors having capsid polypeptides that included an amino acid sequence insert located between amino acid residues 587 and 588 of SEQ ID NO:1 (19 vectors) or an amino acid sequence insert as a replacement of amino acid residues 585 to 590 of SEQ ID NO: 1 (8 vectors) mediated efficient packaging. The AAV vectors were ranked based on overall rankings with +++ indicating those that performed in the top ⅓ of vectors tested, with ++ indicating those that performed in middle ⅓ of vectors tested, and with + indicating those that performed in the bottom ⅓ of vectors tested. SEQ ID NO: 14 (SEQ ID NO:5 inserted between amino acid residues 587 and 588 of SEQ ID NO: 1; see, e.g., FIG. 1) resulted in “+++.”

Taken together, these results demonstrate that AAV vectors that include an AAV capsid polypeptide (e.g., an AAV2 capsid polypeptide) having an amino acid sequence set forth in Table 1 (or Formula A) can have the ability to mediate efficient packaging.

### Example 24—Treating a Retinal Condition Using an AAV Vector

An AAV vector is constructed to include an AAV2 capsid polypeptide having an amino acid sequence set forth in Table 1 (e.g., SEQ ID NO:2 or 5) (or Formula A) and an exogenous nucleic acid sequence encoding a therapeutic polypeptide. The constructed AAV vector is administered intravitreally to a human identified as having a retinal condition in an amount that is from about 1×10⁷ to about 1×10¹⁴ AAV vectors. After the administration, the severity of one or more symptoms of the retinal condition is reduced and/or the progression of the retinal condition is slowed.

### Example 25—AAV Vectors Containing Mutated Capsid Polypeptides

Highly diverse libraries of AAV variants (about 1E+5 to about 1E+6 variants) were pooled and injected into the eyes of rhesus macaques and cynomolgus macaques non-human primates (n=4) via intravitreal injection. The AAVs were packaged with a ubiquitous CMV promoter driving expression of a GFP transgene. Barcodes identifying unique AAV variants were included following the GFP transgene. 30-60 days following injection, single-cell RNA-Seq was used to quantify the expression of GFP as a metric of the performance of variants in the pool. The variant with SEQ ID NO:5 emerged as a top performer from this screen in four non-human primates. Performance was quantified according to the number of cells expressing the transgene.

In a separate experiment, AAV variants including a variant having SEQ ID NO:66 were cloned, packaged, and pooled together. The AAV variants were pooled and injected into the eyes of rhesus macaques and cynomolgus macaques non-human primates (n=3) via intravitreal injection. The AAVs were packaged with a ubiquitous CAG promoter driving expression of a GFP transgene. Barcodes identifying unique AAV variants were included following the GFP transgene. 30-60 days following injection, single-cell RNA-Seq was used to quantify the expression of GFP as a metric of the performance of variants in the pool. AAV2 (Scientific name: Adeno-associated virus 2 (isolate Srivastava/1982); UniProt Taxon ID No. 648242) was included in the mixture as a benchmarking control in the screen. The performance of each variant was quantified according to the number of cells expressing the transgene. The variant containing SEQ ID NO:66 outperformed the naturally occurring serotype controls across all cell types in all non-human primates, and performed similarly to engineered serotypes. Injection of the variant containing SEQ ID NO:5 also resulted in increased levels of transgene expression per cell relative to that observed with the naturally occurring serotypes (Table 4).

TABLE 4

| | AAV2 engineered to | Wild-type |
|---|---|---|
| Cell Type | include SEQ ID NO: 66 | AAV2 |
| Rod | +++++ | nd |
| Cone | nd | nd |
| Horizontal Cell | +++++ | nd |
| Off-Bipolar | +++ | nd |
| On-Bipolar | ++++++ | nd |
| Amacrine Cell | +++++++ | +++++++ |
| Microglia | ++++ | +++++ |
| Muller Glia | ++++++++ | +++++ |
| Retinal Ganglion Cell | +++++++ | nd |
| Retinal pigment epithelium | +++++ | nd |
| All Cells | +++++++ | +++++ |

nd = not detectable.
Plus rankings were based upon log-transformed percent cells infected.

These results demonstrate that AAV vectors with an AAV capsid polypeptide that includes an amino acid sequence set forth in Table 1 (or Formula A) effectively infect retinal cells and result in high level expression of delivered nucleic acid in those infected cells.

### Example 26—Additional Embodiments

Embodiment 1. An AAV capsid polypeptide comprising the amino acid sequence of any one of SEQ ID NOs: 2-5.

Embodiment 2. The polypeptide of Embodiment 1, wherein said polypeptide comprises the amino acid sequence of SEQ ID NO: 1 or SEQ ID NO: 10 except that said amino acid sequence of any one of SEQ ID NOs: 2-5 is located between amino acid positions 587 and 588 of SEQ ID NO:1 or SEQ ID NO:10.

Embodiment 3. The polypeptide of Embodiment 1, wherein said polypeptide comprises the amino acid sequence of SEQ ID NO: 1 or SEQ ID NO: 10 except that said amino acid sequence of SEQ ID NO:5 is located between amino acid positions 587 and 588 of SEQ ID NO: 1 or SEQ ID NO: 10.

Embodiment 4. The polypeptide of Embodiment 1, wherein said polypeptide comprises the amino acid sequence of SEQ ID NO: 1 or SEQ ID NO: 10 except that the amino acids from position 585 to 590 of SEQ ID NO: 1 or SEQ ID NO: 10 are replaced with said amino acid sequence of any one of SEQ ID NOs: 2-5.

Embodiment 5. The polypeptide of Embodiment 1, wherein said polypeptide comprises the amino acid sequence of SEQ ID NO: 1 or SEQ ID NO: 10 except that the amino acids from position 585 to 590 of SEQ ID NO: 1 or SEQ ID NO: 10 are replaced with said amino acid sequence of SEQ ID NO:2.

Embodiment 6. The polypeptide of any one of Embodiments 1-5, wherein an AAV vector comprising said polypeptide infects greater than 2.5 percent of retinal cells when a titer of at least 1×10⁷ of said vector is administered intravitreally to an eye of a human.

Embodiment 7. The polypeptide of any one of Embodiments 1-6, wherein an AAV vector comprising said polypeptide expresses more nucleic acid in retinal cells than the level of expression from a comparable AAV vector comprising a capsid polypeptide consisting of the amino acid sequence set forth in SEQ ID NO:1.

Embodiment 8. A nucleic acid molecule encoding a polypeptide of any one of Embodiments 1-7.

Embodiment 9. The nucleic acid molecule of Embodiment 8, wherein said nucleic acid molecule is DNA.

Embodiment 10. A host cell comprising a nucleic acid molecule of any one of Embodiments 8-9.

Embodiment 11. The host cell of Embodiment 10, wherein said host cell expresses a vector comprising said polypeptide.

Embodiment 12. The host cell of Embodiment 10, wherein said host cell expresses said polypeptide.

Embodiment 13. A host cell comprising a polypeptide of any one of Embodiments 1-7.

Embodiment 14. The host cell of any one of Embodiments 10-13, wherein said host cell is a retinal cell.

Embodiment 15. A non-naturally occurring AAV capsid polypeptide, wherein said capsid polypeptide comprises the amino acid sequence of SEQ ID NO: 1 or SEQ ID NO:10 comprising an amino acid sequence insert of Formula A located between amino acid positions 587 and 588 of SEQ ID NO:1 or SEQ ID NO: 10, wherein said Formula A is:

-L1-EGSGRN (SEQ ID NO:2)-L2-,

wherein said L1 and said L2 are each independently optional amino acid linkers having one, two, or three amino acids.

Embodiment 16. The capsid polypeptide of Embodiment 15, wherein said L1 is one amino acid X1.

Embodiment 17. The capsid polypeptide of Embodiment 17, wherein said X1 is selected from the group of amino acid residues consisting of A, V, I, and L.

Embodiment 18. The capsid polypeptide of Embodiment 17, wherein said X1 is A.

Embodiment 19. The capsid polypeptide of Embodiment 15, wherein said L1 is two amino acids X2-X1.

Embodiment 20. The capsid polypeptide of Embodiment 19, wherein said X1 is selected from the group of amino acid residues consisting of A, V, I, and L.

Embodiment 21. The capsid polypeptide of Embodiment 19, wherein said X1 is A.

Embodiment 22. The capsid polypeptide of any one of Embodiments 19-21, wherein said X2 is selected from the group of amino acid residues consisting of A, V, I, and L.

Embodiment 23. The capsid polypeptide of Embodiment 22, wherein said X2 is L.

Embodiment 24. The capsid polypeptide of Embodiment 19, wherein said X2-X1 is LA.

Embodiment 25. The capsid polypeptide of Embodiment 15, wherein said L1 is three amino acids X3-X2-X1.

Embodiment 26. The capsid polypeptide of Embodiment 25, wherein said X1 is selected from the group of amino acid residues consisting of A, V, I, and L.

Embodiment 27. The capsid polypeptide of Embodiment 26, wherein said XI is A.

Embodiment 28. The capsid polypeptide of any one of Embodiments 25-27, wherein said X2 is selected from the group of amino acid residues consisting of A, V, I, and L.

Embodiment 29. The capsid polypeptide of Embodiment 28, wherein said X2 is L.

Embodiment 30. The capsid polypeptide of Embodiment 25, wherein said X2-X1 is LA.

Embodiment 31. The capsid polypeptide of any one of Embodiments 25-30, wherein said X3 is selected from the group of amino acid residues consisting of A, V, I, and L.

Embodiment 32. The capsid polypeptide of Embodiment 15, wherein said L1 is absent.

Embodiment 33. The capsid polypeptide of any one of Embodiments 15-32, wherein said L2 is one amino acid Z1.

Embodiment 34. The capsid polypeptide of Embodiment 33, wherein said ZI is selected from the group of amino acid residues consisting of A, V, I, and L.

Embodiment 35. The capsid polypeptide of Embodiment 34, wherein said Z1 is A.

Embodiment 36. The capsid polypeptide of any one of Embodiments 15-32, wherein said L2 is two amino acids Z1-Z2.

Embodiment 37. The capsid polypeptide of Embodiment 36, wherein said ZI is selected from the group of amino acid residues consisting of A, V, I, and L.

Embodiment 38. The capsid polypeptide of Embodiment 37, wherein said Z1 is A.

Embodiment 39. The capsid polypeptide of any one of Embodiments 36-38, wherein said Z2 is selected from the group of amino acid residues consisting of A, V, I, and L.

Embodiment 40. The capsid polypeptide of Embodiment 39, wherein said Z2 is L.

Embodiment 41. The capsid polypeptide of Embodiment 36, wherein said Z1-Z2 is AL.

Embodiment 42. The capsid polypeptide of any one of Embodiments 15-32, wherein said L2 is three amino acids Z1-Z2-Z3.

Embodiment 43. The capsid polypeptide of Embodiment 42, wherein said Z1 is selected from the group of amino acid residues consisting of A, V, I, and L.

Embodiment 44. The capsid polypeptide of Embodiment 43, wherein said Z1 is A.

Embodiment 45. The capsid polypeptide of any one of Embodiments 42-44, wherein said Z2 is selected from the group of amino acid residues consisting of A, V, I, and L.

Embodiment 46. The capsid polypeptide of Embodiment 45, wherein said Z2 is L.

Embodiment 47. The capsid polypeptide of Embodiment 42, wherein said Z1-Z2 is AL.

Embodiment 48. The capsid polypeptide of any one of Embodiments 42-47, wherein said Z3 is selected from the group of amino acid residues consisting of A, V, I, and L.

Embodiment 49. The capsid polypeptide of any one of Embodiments 15-32, wherein said L2 is absent.

Embodiment 50. The capsid polypeptide of Embodiment 15, wherein said amino acid sequence insert comprises any one of SEQ ID NOs: 2-5.

Embodiment 51. A viral particle comprising a capsid polypeptide of any one of Embodiments 15-51.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-92. (canceled)

93. An adeno-associated virus (AAV) capsid polypeptide, wherein the AAV capsid polypeptide comprises an amino acid sequence selected from any one of SEQ ID NOs: 2, 3, 4, or 5.

94. The AAV capsid polypeptide of claim 93, wherein the capsid polypeptide comprises the amino acid sequence of any one of SEQ ID NOs: 2-5 located between amino acid residues corresponding to positions 587 and 588 of SEQ ID NO: 1 or SEQ ID NO: 10.

95. The AAV capsid polypeptide of claim 93, wherein the capsid polypeptide comprises the amino acid sequence of SEQ ID NO: 5 located between amino acid residues corresponding to positions 587 and 588 of SEQ ID NO: 1 or SEQ ID NO: 10.

96. The AAV capsid polypeptide of claim 93, wherein the capsid polypeptide has an amino acid sequence comprising the amino acid sequence of any one of SEQ ID NOs: 2-5 replacing amino acid residues corresponding to positions 585 to 590 of SEQ ID NO: 1 or SEQ ID NO: 10.

97. The AAV capsid polypeptide of claim 93, wherein the capsid polypeptide has an amino acid sequence comprising SEQ ID NO: 2 replacing amino acid residues corresponding to positions 585 to 590 of SEQ ID NO: 1 or SEQ ID NO: 10.

98. An AAV particle comprising (i) an AAV capsid polypeptide according to claim 93; and (ii) an exogenous nucleic acid that encodes an RNA or polypeptide.

99. A method of delivering an exogenous nucleic acid to a mammalian retinal cell, the method comprising contacting the retinal cell with an AAV particle comprising acapsid polypeptide of capsid polypeptide according to claim 93 and an exogenous nucleic acid, wherein the AAV particle infects the retinal cell, thereby delivering the exogenous nucleic acid to the retinal cell.

100. The method of claim 99, wherein the capsid polypeptide has an amino acid sequence comprising any one of SEQ ID NOs: 2-5 located (i) between amino acid residues corresponding to positions 587 and 588 of SEQ ID NO: 1 or SEQ ID NO: 10; or (ii) the amino acid residues corresponding to positions 585 to 590 of SEQ ID NO: 1 or SEQ ID NO: 10 are replaced with the amino acid sequence of any one of SEQ ID NOs: 2-5.

101. The method of claim 99, wherein the mammal is a human.

102. A method of treating a retinal condition in a mammal comprising delivering an effective amount of a composition comprising an AAV particle comprising (i) a capsid polypeptide according to claim 93 and (ii) an exogenous nucleic acid sequence, to the mammal, wherein one or more retinal cells is/are infected using the AAV particle, which then drives expression of the exogenous nucleic acid sequence within the retinal cells, thereby treating the retinal condition.

103. The method of claim 102, wherein the capsid polypeptide has an amino acid sequence of any one of SEQ ID NOs: 2-5 is located (i) between amino acid residues corresponding to positions 587 and 588 of SEQ ID NO: 1 or SEQ ID NO: 10; or (ii) the amino acid residues corresponding to positions 585 to 590 of SEQ ID NO: 1 or SEQ ID NO: 10 are replaced with the amino acid sequence of any one of SEQ ID NOs: 2-5.

104. The method of claim 102, wherein the mammal is a human.

105. The method of claim 102, wherein the one or more infected retinal cell or cells expresses more of the exogenous nucleic acid sequence than the level of expression in a retinal cell from a comparable AAV particle comprising a capsid polypeptide consisting of the amino acid sequence set forth in SEQ ID NO: 1.

106. The method of claim 102, wherein the retinal condition is selected from LCA, OCA1, retinitis pigmentosa, rod/cone dystrophy, cone dystrophy, Stargardt Disease, Usher syndrome, XLRP, and/or XLRS.

107. An adeno-associated virus (AAV) particle comprising an AAV capsid polypeptide, wherein the capsid polypeptide has an amino acid sequence comprising an amino acid sequence insert of Formula A located between amino acid residues corresponding to positions 587 and 588 of SEQ ID NO: 1 or SEQ ID NO: 10, wherein Formula A is:
(SEQ ID NO: 2)
-L1-EGSGRN -L2-,
wherein L1 and L2 are each independently optional amino acid linkers having zero, one, two, or three amino acids.

108. The AAV particle of claim 107, wherein L1 is:
(i) absent;
(ii) one amino acid X1, and X1 is selected from the group of amino acid residues consisting of A, V, I, and L;
(iii) two amino acids X2-X1, wherein X1 is selected from the group of amino acid residues consisting of A, V, I, and L, and X2 is selected from the group of amino acid residues consisting of A, V, I, and L; or
(iv) three amino acids X3-X2-X1, wherein X1 is selected from the group of amino acid residues consisting of A, V, I, and L, X2 is selected from the group of amino acid residues consisting of A, V, I, and L, and X3 is selected from the group of amino acid residues consisting of A, V, I, and L.

109. The AAV particle of claim 107, wherein L2 is:
(i) absent;
(ii) one amino acid Z1, wherein Z1 is selected from the group of amino acid residues consisting of A, V, I, and L;
(iii) two amino acids Z1-Z2, wherein Z1 is selected from the group of amino acid residues consisting of A, V, I, and L, and Z2 is selected from the group of amino acid residues consisting of A, V, I, and L; or
(iv) three amino acids Z1-Z2-Z3, wherein Z1 is selected from the group of amino acid residues consisting of A, V, I, and L, Z2 is selected from the group of amino acid residues consisting of A, V, I, and L, and Z3 is selected from the group of amino acid residues consisting of A, V, I, and L.

110. The AAV particle of claim 107, wherein L1 is:
(i) absent;
(ii) one amino acid X1, and X1 is selected from the group of amino acid residues consisting of A, V, I, and L;
(iii) two amino acids X2-X1, wherein X1 is selected from the group of amino acid residues consisting of A, V, I, and L, and X2 is selected from the group of amino acid residues consisting of A, V, I, and L; or
(iv) three amino acids X3-X2-X1, wherein X1 is selected from the group of amino acid residues consisting of A, V, I, and L, X2 is selected from the group of amino acid residues consisting of A, V, I, and L, and X3 is selected from the group of amino acid residues consisting of A, V, I, and L; and/or
wherein L2 is:
(v) absent;
(vi) one amino acid Z1, wherein Z1 is selected from the group of amino acid residues consisting of A, V, I, and L;
(vii) two amino acids Z1-Z2, wherein Z1 is selected from the group of amino acid residues consisting of A, V, I, and L, and Z2 is selected from the group of amino acid residues consisting of A, V, I, and L; or
(viii) three amino acids Z1-Z2-Z3, wherein Z1 is selected from the group of amino acid residues consisting of A, V, I, and L, Z2 is selected from the group of amino acid residues consisting of A, V, I, and L, and Z3 is selected from the group of amino acid residues consisting of A, V, I, and L.

111. The AAV particle of claim 107, wherein the amino acid sequence insert comprises any one of SEQ ID NOs: 2-5.

112. An AAV capsid polypeptide, wherein the capsid polypeptide has an amino acid sequence insert of Formula A located between amino acid residues corresponding to positions 587 and 588 of SEQ ID NO: 1 or SEQ ID NO: 10, wherein Formula A is:
(SEQ ID NO: 2)
-L1-EGSGRN -L2-,
wherein L1 and said L2 are each independently optional amino acid linkers having zero, one, two, or three amino acids.

113. An AAV particle comprising a capsid polypeptide according to claim 93, wherein the amino acid sequence of any one of SEQ ID NOs: 2-5 is located between amino acid residues corresponding to positions 587 and 588 of SEQ ID NO: 1 or SEQ ID NO: 10.

114. An AAV vector comprising a polynucleotide encoding the AAV capsid polypeptide of claim 93.

115. The AAV vector of claim 114, wherein the nucleic acid sequence encoding any one of SEQ ID NOs: 2-5 is located between a nucleic acid sequence encoding amino acid residues corresponding to positions 587 and 588 of SEQ ID NO: 1 or SEQ ID NO: 10.

116. A mammalian cell comprising the AAV vector of claim 114.

117. A mammalian cell comprising a first AAV vector according to claim 114, and a second AAV vector comprising an exogenous polynucleotide that encodes an RNA or a polypeptide.

118. An AAV particle produced using a mammalian cell comprising an AAV vector of claim 114.

119. An adeno-associated virus (AAV) capsid polypeptide, wherein the AAV capsid polypeptide comprises an amino acid sequence selected from any one of SEQ ID NOs: 2, 3, 4, or 5, wherein the amino acid sequence of the any one of SEQ ID NOs: 2-5 is located between residues corresponding to positions 587 and 588 of SEQ ID NO: 1 or SEQ ID NO: 10.
